(19) United States
(12) Patent Application Publication
(10) Pub. No.: US 2008/0107661 A1
(43) Pub. Date: May 8, 2008

(54) MUC1 and ABL

(75) Inventor: Donald W. Kufe, Wellesley, MA (US)

Correspondence Address: FISH & RICHARDSON P.C., P.O. BOX 1022, MINNEAPOLIS, MN, 55440-1022, US

(73) Assignee: Dana-Farber Cancer Institute, Inc., Boston, MA (US)

(21) Appl. No.: 11/829,687

(22) Filed: Jul. 27, 2007

Related U.S. Application Data
(60) Provisional application No. 60/834,018, filed on Jul. 28, 2006.

(51) Int. Cl. A61K 38/00; C12Q 1/48; G06G 7/48; A61K 31/195; A61P 43/00; C12N 5/06; A61K 39/395

(52) U.S. Cl. 424/172.1; 435/15; 703/11; 435/375; 514/2; 514/567

(57) ABSTRACT

The present disclosure provides methods of identifying and making compounds and pharmaceutical compositions thereof that inhibit the interaction between MUC1 and Abl. The invention also provides in vivo, in vitro, and ex vivo methods of inhibiting such an interaction. Also featured are in vitro and in vivo methods of stimulating the Abl-dependent apoptotic pathway in cells expressing MUC1. In such methods, the compounds, compositions, and methods described herein are generally useful in the treatment of various cancers. The disclosure also provides methods for inhibiting Abl, and such methods, and compounds and compositions for use in the methods are generally useful for the treatment of cancers, inflammatory conditions, atherosclerotic lesions, and neurologic disorders.

### CROSS-REFERENCE TO RELATED APPLICATIONS

This application claims priority to U.S. Provisional Application No. 60/834,018, filed on Jul. 28, 2006, the disclosure of which is hereby incorporated by reference in its entirety.

### STATEMENT REGARDING FEDERALLY SPONSORED RESEARCH OR DEVELOPMENT

The research described in this application was supported by two grants from the National Cancer Institute of the National Institutes of Health (CA097098 and CA29431) and a grant from the U.S. Army (BC022158). Thus, the government has certain rights in the invention.

### TECHNICAL FIELD

This invention relates to regulation of cell signaling, cell growth, and more particularly to the regulation of cancer cell growth.

### BACKGROUND

DNA damage activates a nuclear complex that consists in part of the nonreceptor c-Abl tyrosine kinase (Kharbanda et al., (1995) Nature 376:785-788; Yuan et al., (1997) Proc. Natl. Acad. Sci. USA 94:1437-1440). c-Abl binds to the p53 tumor suppressor in the response to genotoxic stress (Yuan et al., (1996) J. Biol. Chem. 271:26457-26460); Yuan et al., (1996) Nature 382:272-274) and regulates p53 by preventing its nuclear export (Sionov et al., (2001) Mol. Cell. Biol. 21:5869-5878). Other studies have demonstrated that c-Abl interacts with the p73 homolog of p53 in the apoptotic response of cells to DNA damage (Agarni et al., (1999) Nature 399:809-813); Gong et al., (1999) Nature 399:806-809; Yuan et al., (1999) Nature 399:814-817). Nuclear c-Abl also activates MEK kinase-1 and thereby the pro-apoptotic c-Jun N-terminal kinase (JNK) pathway (Kharbanda et al., (1995) J. Biol. Chem. 270:30278-30281; Kharbanda et al., (2000) Mol. Cell. Biol. 20:4979-4989; Kharbanda et al., (1995) Nature 376:785-788). c-Abl contains three nuclear localization signals and one nuclear export signal in the carboxy-terminal region that are responsible for shuttling of c-Abl between the cytoplasm and nucleus (Taagepera et al., (1998) Proc. Natl. Acad. Sci. USA 95:7457-7462; Wen et al., (1996) EMBO J. 15:1583-1595). Recent work has shown that c-Abl is sequestered in the cytoplasm through binding to 14-3-3 proteins (Yoshida et al., (2005) Nat. Cell. Biol. 7:278-285). Phosphorylation of c-Abl on Thr-735 by an as yet unidentified kinase serves as a site for direct binding to 14-3-3. In the response to DNA damage, activation of JNK induces phosphorylation of 14-3-3 and the release of c-Abl for targeting to the nucleus (Yoshida et al., (2005) Nat. Cell. Biol. 7:278-285). In this regard, expression of a 14-3-3 mutant at the JNK phosphorylation site attenuates DNA damage-induced nuclear import of c-Abl and apoptosis.

The MUC1 transmembrane glycoprotein is expressed as a stable heterodimer following synthesis as a single polypeptide and cleavage into two subunits in the endoplasmic reticulum (Ligtenberg et al., (1992) J. Biol. Chem. 267:6171-6177). MUC1 is normally localized to the apical borders of secretory epithelial cells (Kufe et al., (1984) Hybridoma 3:223-232). With transformation and loss of polarity, the MUC1 heterodimer is aberrantly overexpressed on the entire cell membrane (Kufe et al., (1984) Hybridoma 3:223-232). The MUC1 N-terminal subunit (MUC1-N) contains variable numbers of 20 amino acid tandem repeats that are modified by O-linked glycans (Gendler et al., (1988) J. Biol. Chem. 263:12820-12823; Siddiqui et al., (1988) Proc. Natl. Acad. Sci. USA 85:2320-2323). MUC1-N is tethered to the cell membrane by the MUC1 C-terminal subunit (MUC1-C), which includes a transmembrane region and a 72 amino acid cytoplasmic domain (MUC1-CD) (Merlo et al., (1989) Cancer Res. 49:6966-6971). MUC1-CD associates with β-catenin (Huang et al., (2005) Cancer Res. 65:10413-10422; Li et al., (1998) Mol. Cell. Biol. 18:7216-7224; Yamamoto et al., (1997) J. Biol. Chem. 272:12492-12494) and the p53 tumor suppressor (Wei et al., (2005) Cancer Cell 7:167-178). In addition, MUC1-CD is phosphorylated by the epidermal growth factor receptor (EGFR) (Li et al., (2001) J. Biol. Chem. 276:35239-35242), c-Src (Li et al., (2001) J. Biol. Chem. 276:6061-6064) and glycogen synthase kinase 3β (GSK3β) (Li et al., (1998) Mol. Cell. Biol. 18:7216-7224). Other studies have shown that overexpression of MUC1 confers transformation (Huang et al., (2005) Cancer Res. 65:10413-10422; Li et al., (2003) Oncogene 22:6107-6110) and blocks the apoptotic response to genotoxic stress (Raina et al., (2004) J. Biol. Chem. 279:20607-20612; Ren et al., (2004) Cancer Cell 5:163-175). In this regard, and in addition to expression at the cell membrane, MUC1-C localizes to the mitochondrial outer membrane and inhibits DNA damage-induced release of apoptogenic factors (Ren et al., (2004) Cancer Cell 5:163-175; Ren et al., (2006) Oncogene 25:20-31). Mitochondrial targeting of MUC1-C is attenuated by mutation of the cytoplasmic domain at Tyr-46 (Ren et al., (2004) Cancer Cell 5:163-175; Ren et al., (2006) Oncogene 25:20-31). Moreover, expression of MUC1 with a Y46F mutation attenuates the anti-apoptotic function of MUC1 in the response to DNA damage (Ren et al., (2004) Cancer Cell 5:163-175).

### SUMMARY

This invention is based, at least in part, on the discovery that MUC1 (mucin 1) is involved in regulating the DNA-damage response signal-transduction pathway in mammalian cells through an interaction with c-Abl kinase. MUC1 binds to c-Abl, prevents its phosphorylation, and sequesters the kinase from the nucleus, thereby preventing normal induction of apoptosis following genotoxic stress. In this way, MUC1 promotes cell survival (e.g., cancer cell survival). Thus, inhibition of MUC1 can be useful in inhibiting the growth of cells, particularly cancer cells. Such methods of inhibiting the growth of cancer cells and compounds and compositions useful in the methods are described herein. In addition, for conditions in which inhibiting the activity of Abl can be beneficial (e.g., neurologic disorders such as Alzheimer's Disease and Parkinson's Disease), Abl-binding fragments of MUC1 capable of binding to and inhibiting an Abl polypeptide and methods of use are also provided.

In one aspect, the disclosure features a method of identifying a compound that inhibits the binding of Abl to MUC1 (mucin 1). The method includes the steps of: providing a MUC1 test agent; providing an Abl test agent that binds to the MUC1 test agent; contacting the MUC1 test agent with the Abl test agent in the presence of a test compound; and determining whether the test compound inhibits binding of the MUC1 test agent to the Abl test agent. The method can be performed (i.e., carried out) in a cell or in a cell-free system. In embodiments where the method is carried out in a cell, cells suitable for the method can be any prokaryotic cell (e.g., a bacterial cell) or eukaryotic cell (e.g., a yeast cell, a nematode cell, an insect cell, a bird cell, a mammalian cell (e.g., a mouse cell, a rat cell, a guinea pig cell, a horse cell, a cow cell, a pig cell, a goat cell, a donkey cell, a monkey cell, or a human cell)). MUC1 test agents can include any agent containing a full-length, wild-type, mature MUC1 or the MUC1-cytoplasmic domain (MUC1-CD) (SEQ ID NO:2), or fragments (e.g., functional fragments) of the full-length, wild-type, MUC1 or MUC1-CD. Fragments of the MUC1-CD can also include any fragments containing the YTNP motif (SEQ ID NO:3) at positions 60-63 of SEQ ID NO:2. In some embodiments, the tyrosine of the YTNP motif can be phosphorylated, and the resulting phosphorylated motif is hereinafter referred to as pYTNP. Fragments including the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The Abl test agent can include: (a) full-length, wild-type c-Abl; (b) a drug or multidrug-resistant variant of c-Abl; (c) BCR-Abl; (d) a functional fragment of (a), (b), or (c); or (e) (a), (b), (c), or (d) with not more than 50 (see below) conservative substitutions. Abl can be c-Abl, v-Abl, BCR-Abl, or any of numerous drug and multi-drug resistant forms of Abl. The Abl test agent can be an Abl polypeptide from any species (e.g., nematode, insect, plant, bird, reptile, or mammal (e.g., a mouse, rat, dog, cat, goat, pig, cow, horse, whale, or monkey) that expresses a homolog of a human Abl protein. The Abl fragment can include the SH2 domain of c-Abl (SEQ ID NO:5) or a MUC1-binding fragment of the SH2 domain. The Abl fragment can also include the SH3 domain of c-Abl (SEQ ID NO:8) or a MUC1-binding fragment of the SH3 domain.

In another aspect, the disclosure features a method of generating a compound that inhibits the interaction between MUC1 and an Abl polypeptide. The method includes the steps of: providing the three-dimensional structure of a molecule comprising (a) the cytoplasmic domain of MUC1 or an Abl polypeptide-binding fragment thereof, or (b) a molecule comprising an Abl polypeptide or MUC1-binding fragment thereof; designing, based on the three dimensional structure, a compound comprising a region that inhibits the interaction between MUC1 and the Abl polypeptide; and producing the compound. The method can further include the step of determining whether the compound identified by the method inhibits the interaction between MUC1 and an Abl polypeptide. Abl-binding fragments of MUC can include the MUC1-CD (SEQ ID NO:2), or fragments (e.g., functional fragments) of the MUC1-CD containing the YTNP (SEQ ID NO:3) motif at positions 60-63 of SEQ ID NO:2. Fragments of the MUC1-CD containing the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The invention also features a compound identified by the method, and a pharmaceutical composition containing the compound and a pharmaceutically acceptable carrier.

In yet another aspect, the disclosure features an in vitro method of inhibiting an interaction between MUC1 and Abl. The method includes: optionally identifying a cell as expressing MUC1; and culturing the cell with a compound that inhibits an interaction between MUC1 and Abl. The cell can be a mammalian cell (e.g., a mouse cell, a rat cell, a guinea pig cell, a horse cell, a cow cell, a pig cell, a goat cell, a donkey cell, a monkey cell, or a human cell (e.g., a cell from a human patient)). The cell can be a normal cell or a cell from a cancer cell line (e.g., a colon cancer cell line or breast cancer cell line). The cell can also be a cancer cell from a cancer including, but not limited to: lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer. Where a cell is identified as a cell expressing MUC1, the expression can include the expression of MUC1 mRNA or MUC1 protein by or in the cell. The Abl can be any full-length, wild-type c-Abl, BCR-Abl, v-Abl, or drug or multi-drug resistant forms of an Abl polypeptide. The compound can be a compound identified in any of the methods for generating a compound above or can be any compound with appropriate inhibitory activity. In some embodiments, the method can further include the step of culturing the cell with (i.e., co-culturing the cell with a compound described above and also with) one or more additional genotoxic agents or chemotherapeutic agents, one or more forms of ionizing radiation, or one or more kinase inhibitors. The one or more genotoxic and chemotherapeutic agents can be, but are not limited to, one or more of carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to: one or more of gamma-radiation, X-irradiation, infrared, UV-radiation, or beta-radiation. The one or more kinase inhibitors can include, but are not limited to, one or more of trastuzumab (e.g., Herceptin®), gefitinib (e.g., Iressa®), erlotinib (e.g., Tarceva®), imatinib mesylate (e.g., Gleevec®), or sunitinib malate (e.g., Sutent®).

In another aspect, the disclosure features an in vitro method of inhibiting the phosphorylation of MUC1 by Abl kinase. The method includes the steps of: optionally identifying a cell as expressing MUC1, and culturing the cell with a compound that inhibits the phosphorylation of MUC1 by Abl kinase. The cell can be a mammalian cell (e.g., a mouse cell, a rat cell, a guinea pig cell, a horse cell, a cow cell, a pig cell, a goat cell, a donkey cell, a monkey cell, or a human cell (e.g., a cell from a human patient)). The cell can be a normal cell or a cell from a cancer cell line (e.g., a colon cancer cell line or breast cancer cell line). The cell can also be a cancer cell from a cancer including, but not limited to: lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer. Where a cell is identified as a cell expressing MUC1, the expression can include the expression of MUC1 mRNA or MUC1 protein by or in the cell. The Abl can be any full-length, wild-type c-Abl, BCR-Abl, v-Abl, or drug or multi-drug resistant forms of an Abl polypeptide. The compound can be a compound identified in any of the methods for generating a compound above, or it can be any compound with appropriate inhibitory activity. In some embodiments, the method can further include the step of culturing the cell with (i.e., co-culturing the cell with a compound described above and also with) one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®).

In another aspect, the disclosure features an in vitro method of inducing apoptosis in a cell. The method includes the steps of optionally identifying a cell as expressing MUC1 and Abl; and culturing the cell with a compound that inhibits the expression of MUC1. The cell can be a mammalian cell (e.g., a mouse cell, a rat cell, a guinea pig cell, a horse cell, a cow cell, a pig cell, a goat cell, a donkey cell, a monkey cell, or a human cell (e.g., a cell from a human patient)). The cell can be a normal cell or a cell from a cancer cell line (e.g., a colon cancer cell line or breast cancer cell line). The cell can also be a cancer cell from a cancer including, but not limited to: lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer. Where a cell is identified as a cell expressing MUC1 and Abl, the expression can include the expression of MUC1 mRNA or MUC1 protein by or in the cell. The Abl can be any full-length, wild-type c-Abl, BCR-Abl, v-Abl, or drug or multi-drug resistant forms of an Abl polypeptide. The compound can be a compound identified in any of the methods for generating a compound above, or it can be any compound with appropriate inhibitory activity. In some embodiments, the method can further include the step of culturing the cell with one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®).

In another aspect, the disclosure features an in vivo method of inhibiting an interaction between MUC1 and Abl. The method involves the steps of: optionally identifying a subject as having, suspected of having, or at risk of developing, a cancer comprising one or more cells expressing MUC1; and delivering to the subject a compound that inhibits an interaction between MUC1 and Abl. Where the one or more cells are identified as one or more cells expressing MUC1, the expression can include the expression of MUC1 mRNA or MUC1 protein by the one or more cells. The Abl can be any full-length, wild-type c-Abl, BCR-Abl, v-Abl, or drug or multi-drug resistant forms of an Abl polypeptide. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. In some embodiments, compounds useful in the method can include Abl-binding fragments of MUC1 (e.g., the MUC1-CD (SEQ ID NO:2) or fragments of the MUC1-CD containing the YTNP motif (SEQ ID NO:3) at positions 60-63 of SEQ ID NO:2). Fragments useful as compounds in the method also can include fragments of the MUC1-CD containing tyrosine 60 of MUC1-CD, where the tyrosine 60 is phosphorylated (e.g., fragments that contain the pYTNP motif). The fragments that include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be a MUC1-binding fragment of c-Abl, e.g., the SH2 domain of human c-Abl (SEQ ID NO:5) or a MUC1-binding fragment of the SH2 domain of human c-Abl, or the SH3 domain of human c-Abl (SEQ ID NO:8) or a MUC1-binding fragment of the SH3 domain. The compound can also be delivered to the subject as a pharmaceutical composition containing the compound and a pharmaceutically acceptable carrier. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to: one or more of carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species of the subject, or from the same subject, to be treated with the cell. The subject can be any mammal, e.g., a mouse, a rat, a guinea pig, a horse, a cow, a pig, a goat, a donkey, a monkey, or a human (e.g., a human patient). The cancer can be, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In another aspect, the disclosure provides an in vivo method of inhibiting phosphorylation of MUC1 by Abl kinase. The method includes the steps of: optionally identifying a subject as having, suspected of having, suspected of having, or at risk of developing, a cancer comprising one or more cells expressing MUC1; and delivering to the subject a compound that inhibits the phosphorylation of MUC1 by Abl kinase. Where the one or more cells are identified as one or more cells expressing MUC1, the expression can include the expression of MUC1 mRNA or MUC1 protein by the one or more cells. The Abl kinase can be any full-length, wild-type c-Abl, BCR-Abl, v-Abl, or drug or multi-drug resistant forms of an Abl polypeptide, or any functional fragment of an Abl polypeptide that retains at least 10% (e.g., at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99% or more) of the kinase activity of the parent Abl polypeptide from which the functional fragment was derived. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. In some embodiments, compounds useful in the method can include Abl-binding fragments of MUC1 (e.g., the MUC1-CD (SEQ ID NO:2), or fragments of the MUC1-CD containing the YTNP motif (SEQ ID NO:3) at positions 60-63 of SEQ ID NO:2). Fragments useful as compounds in the method also can include fragments of the MUC1-CD containing tyrosine 60 of MUC1-CD, where the tyrosine 60 is phosphorylated (e.g., fragments containing the pYTNP motif). The fragments can also include the YTNP motif, and can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be a MUC1-binding fragment of c-Abl, e.g., the SH2 domain of human c-Abl (SEQ ID NO:5) or a MUC1-binding fragment of the SH2 domain of human c-Abl, or the SH3 domain of human c-Abl (SEQ ID NO:8) or a MUC1-binding fragment of the SH3 domain of human c-Abl. The compound can also be delivered to the subject as a pharmaceutical composition containing the compound and a pharmaceutically acceptable carrier. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject of the method can be any mammal, e.g., a mouse, a rat, a guinea pig, a horse, a cow, a pig, a goat, a donkey, a monkey, or a human (e.g., a human patient). The cancer can be, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In yet another aspect, the disclosure features an in vivo method of inhibiting BCR-Abl. The method includes the steps of: optionally identifying a subject as having, suspected of having, or at risk of developing, a cancer comprising one or more cells expressing MUC-1; and delivering to the subject a compound that inhibits BCR-Abl. Inhibition of BCR-Abl can include inhibition of BCR-Abl kinase activity or inhibition of BCR-Abl expression. Inhibition of BCR-Abl expression can include inhibition of the expression of BCR-Abl mRNA or protein by the one or more cells. Where the one or more cells are identified as one or more cells expressing MUC1, the expression can include the expression of MUC1 mRNA or MUC1 protein by the one or more cells. The BCR-Abl kinase can be any form of BCR-Abl fusion protein, a drug or multi-drug resistant form of a BCR-Abl polypeptide, or any functional fragment of a BCR-Abl polypeptide that retains at least 10% (e.g., at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99% or more) of the kinase activity of BCR-Abl protein. The compound can be a compound identified in any of the methods for generating a compound above, or it can be any compound with appropriate inhibitory activity (e.g., imatinib mesylate (e.g., Gleevec®)). The compounds can be, e.g., a small molecule (including an aptamer), an antibody, an antibody fragment, a polypeptide, or a peptidomimetic. In some embodiments, compounds useful in the method can include BCR-Abl-binding fragments of MUC1 (e.g., the MUC1-CD (SEQ ID NO:2), or fragments of the MUC1-CD containing the YTNP motif (SEQ ID NO:3) at positions 60-63 of SEQ ID NO:2). In some embodiments, the tyrosine of the YTNP motif can be phosphorylated. Fragments useful as compounds in the method also can include fragments of the MUC1-CD containing tyrosine 60 of MUC1-CD, where the tyrosine 60 is phosphorylated (e.g., fragments containing the pYTNP motif). The fragments that include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be a MUC1-binding fragment of c-Abl or BCR-Abl, e.g., the SH2 domain of human c-Abl (SEQ ID NO:5) or a MUC1-binding fragment of the SH2 domain of human c-Abl, or the SH3 domain of human c-Abl (SEQ ID NO:8) or a MUC1-binding fragment of the SH3 domain of human c-Abl. The compound can also be delivered to the subject as a pharmaceutical composition containing the compound and a pharmaceutically acceptable carrier. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent°). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject of the method can be any mammal, e.g., a mouse, a rat, a guinea pig, a horse, a cow, a pig, a goat, a donkey, a monkey, or a human (e.g., a human patient). The cancer can be, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In another aspect, the disclosure provides an in vivo method of inducing apoptosis in a cancer cell. The method includes: optionally identifying a subject as having, suspected of having, suspected of having, or at risk of developing, a cancer comprising one or more cells expressing MUC1 and Abl; and delivering to the subject a compound that inhibits the expression of MUC1. Inhibition of MUC1 expression can include inhibition of the expression of MUC1 mRNA or protein by the one or more cells. Where the one or more cells are identified as one or more cells expressing MUC1 and/or Abl, the expression of MUC1 and/or Abl can include the expression of their mRNA or protein by the one or more cells. The compound of the method can be a compound identified in any of the methods for generating a compound above, or it can be any compound with appropriate inhibitory activity (e.g., siRNA or anti-sense oligonucleotides). The compounds can be, e.g., a small molecule (including an aptamer), an antibody, an antibody fragment, a polypeptide, or a peptidomimetic. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject can be any of the subjects described in the method.

In another aspect, the disclosure features an in vivo method of inhibiting an Abl, the method including the steps of: optionally identifying a subject as having, suspected of having, suspected of having, or at risk of developing, a cancer comprising one or more cells expressing Abl; and delivering to the subject a compound comprising a fragment of MUC1 capable of binding to or inhibiting Abl. Inhibition of Abl can include inhibition of Abl kinase activity or inhibition of Abl expression. Inhibition of Abl expression can include inhibition of the expression of Abl mRNA or protein by the one or more cells. Where the one or more cells are identified as one or more cells expressing Abl, the expression can include the expression of Abl mRNA or protein by the one or more cells. The Abl can be any full-length, wild-type c-Abl, BCR-Abl, v-Abl, or drug or multi-drug resistant forms of an Abl polypeptide, or any functional fragment of an Abl polypeptide that retains at least 10% (e.g., at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99% or more) of the kinase activity of the parent Abl polypeptide from which the functional fragment was derived. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. In some embodiments, compounds useful in the method can include Abl-binding fragments of MUC1 (e.g., the MUC1-CD (SEQ ID NO:2) or fragments of the MUC1-CD containing the YTNP motif (SEQ ID NO:3) at positions 60-63 of SEQ ID NO:2). Fragments useful as compounds in the method also can include fragments of the MUC1-CD containing tyrosine 60 of MUC1-CD, where the tyrosine 60 is phosphorylated (e.g., fragments of the MUC1-CD that contain the pYTNP motif). The fragments can also include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be delivered to the subject as a pharmaceutical composition containing the compound and a pharmaceutically acceptable carrier. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject of the method can be any mammal, e.g., a mouse, a rat, a guinea pig, a horse, a cow, a pig, a goat, a donkey, a monkey, or a human (e.g., a human patient). The cancer can be, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In another aspect, the disclosure provides an in vivo method of stimulating translocation of c-Abl to cell nuclei. The method includes the steps of: optionally identifying a subject as having, suspected of having, or at risk of developing, a cancer comprising one or more cells expressing MUC1; and delivering to the subject a compound that stimulates translocation of c-Abl to cell nuclei. Where the one or more cells are identified as one or more cells expressing MUC1, the expression can include the expression of MUC1 mRNA or MUC1 protein by the one or more cells. The Abl can be any full-length, wild-type c-Abl, BCR-Abl, v-Abl, or drug or multi-drug resistant forms of an Abl polypeptide. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. The compound can also be a nucleic acid, e.g., an siRNA or anti-sense nucleic acid (e.g., a MUC1-specific siRNA or anti-sense oligonucleotide). In some embodiments, compounds useful in the method can include Abl-binding fragments of MUC1 (e.g., the MUC1-CD (SEQ ID NO:2), or fragments of the MUC1-CD containing the YTNP motif (SEQ ID NO:3) at positions 60-63 of SEQ ID NO:2). In some embodiments, the tyrosine of the YTNP motif can be phosphorylated. Fragments useful as compounds in the method also can include fragments of the MUC1-CD containing tyrosine 60 of MUC1-CD, where the tyrosine 60 is phosphorylated (e.g., fragments of the MUC1-CD containing the pYTNP motif). The fragments that include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be a MUC1-binding fragment of c-Abl, e.g., the SH2 domain of human c-Abl (SEQ ID NO:5) or a MUC1-binding fragment of the SH2 domain of human c-Abl, or the SH3 domain of human c-Abl (SEQ ID NO:8) or a MUC 1-binding fragment of the SH3 domain of human c-Abl. The compound can also be delivered to the subject as a pharmaceutical composition containing the compound and a pharmaceutically acceptable carrier. In some embodiments, the method can further include the step of delivering to the subject one or more additional genotoxic agents or chemotherapeutic agents, one or more forms of ionizing radiation, or one or more kinase inhibitors. The one or more genotoxic and chemotherapeutic agents can be, but are not limited to, one or more of: carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatin (CDDP), adriamycin (ADR), or an analog of any of the aforementioned. The one or more forms of ionizing radiation can include, but are not limited to, one or more of gamma-radiation, X-irradiation, infrared, UV-radiation, or beta-radiation. The one or more kinase inhibitors can include, but are not limited to, one or more of: trastuzumab (e.g., Herceptin®), gefitinib (e.g., Iressa®), erlotinib (e.g., Tarceva®), imatinib mesylate (e.g., Gleevec®), or sunitinib malate (e.g., Sutent®). The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject of the method can be any mammal, e.g., a mouse, a rat, a guinea pig, a horse, a cow, a pig, a goat, a donkey, a monkey, or a human (e.g., a human patient). The cancer can be, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In another aspect, the disclosure provides an in vivo method of treating a neurologic disorder. The method includes the steps of: optionally identifying a subject as having, or suspected of having, or at risk of developing, a neurologic disorder, where one or more neuronal cells involved in the neurologic disorder express Abl; and delivering to the subject a compound comprising a fragment of MUC1 capable of binding to or inhibiting Abl kinase. Inhibition of Abl can include inhibition of Abl kinase activity or inhibition of Abl expression. Inhibition of Abl expression can include inhibition of the expression of Abl mRNA or protein by the one or more cells. Where the one or more cells are identified as one or more cells expressing Abl, the expression can include the expression of Abl mRNA or protein by the one or more cells. The Abl can be any full-length, wild-type c-Abl, BCR-Abl, v-Abl, or drug or multi-drug resistant forms of an Abl polypeptide, or any functional fragment of an Abl polypeptide that retains at least 10% (e.g., at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99% or more) of the kinase activity of the parent Abl polypeptide from which the functional fragment was derived. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. In some embodiments, the compound can be a nucleic acid, e.g., an siRNA or antisense oligonucleotide (e.g., an Abl-specific siRNA or antisense oligonucleotide). In some embodiments, compounds useful in the method can include Abl-binding fragments of MUC1 (e.g., the MUC1-CD (SEQ ID NO:2), or fragments of the MUC1-CD containing the YTNP motif (SEQ ID NO:3) at positions 60-63 of SEQ ID NO:2). Fragments useful as compounds in the method also can include fragments of the MUC1-CD containing tyrosine 60 of MUC1-CD, where the tyrosine is phosphorylated (e.g., fragments of the MUC1-CD containing the pYTNP motif). The fragments that include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be delivered to the subject as a pharmaceutical composition containing the compound and a pharmaceutically acceptable carrier. The method can further include the step of delivering to the subject one or more additional therapies useful in treating a neurologic disorder. The one or more additional therapies can include administering to the subject: tacrine (Cognex®), donepezil (Aricept®), rivastigmine (Exelon®), galantamine (Razadyne®), or vitamin E. The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject can be any mammal, e.g., a mouse, a rat, a guinea pig, a horse, a cow, a pig, a goat, a donkey, a monkey, or a human (e.g., a human patient). The neurologic disorder can be, e.g., Parkinson's disease, Alzheimer's Disease or another tauopathy (e.g., Pick's disease, progressive supranuclear palsy (PSP), frontotemporal dementia (FTD), or corticobasal degeneration (CBD)).

In yet another aspect, the disclosure features an in vivo method of treating a diabetes-associated atherosclerotic lesion. The method includes optionally identifying a subject, having, suspected of having, or at risk of developing, a diabetes-associated atherosclerotic lesion, wherein the atherosclerotic lesion comprises one or more cells expressing Abl; and delivering to the subject a compound comprising a MUC1 fragment capable of binding to or inhibiting Abl. Inhibition of Abl can include inhibition of Abl kinase activity or inhibition of Abl expression. Inhibition of Abl expression can include inhibition of the expression of Abl mRNA or protein by the one or more cells. Where the one or more cells are identified as one or more cells expressing Abl, the expression can include the expression of Abl mRNA or MUC1 protein by the one or more cells. The Abl can be any full-length, wild-type c-Abl, BCR-Abl, v-Abl, or drug or multi-drug resistant forms of an Abl polypeptide, or any functional fragment of an Abl polypeptide that retains at least 10% (e.g., at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99% or more) of the kinase activity of the wild-type c-Abl protein. The compound can be a compound identified in any of the methods for generating a compound above, or it can be any compound with appropriate inhibitory activity. The compounds can be, e.g., a small molecule (including an aptamer), an antibody, an antibody fragment, a polypeptide, or a peptidomimetic. In some embodiments, the compound can be a nucleic acid, e.g., an siRNA or antisense oligonucleotide (e.g., an Abl-specific siRNA or antisense oligonucleotide). In some embodiments, compounds useful in the method can include Abl-binding fragments of MUC1 (e.g., the MUC1-CD (SEQ ID NO:2), or fragments of the MUC1-CD containing the YTNP motif (SEQ ID NO:3) at positions 60-63 of SEQ ID NO:2). Fragments useful as compounds in the method also can include fragments of the MUC1-CD containing tyrosine 60 of MUC1-CD, where the tyrosine 60 is phosphorylated (e.g., fragments of the MUC1-CD containing the pYTNP motif). The fragments that include the pYTNP motif can range in size. They can be, e.g., amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70 of SEQ ID NO:2. The compound can also be delivered to the subject as a pharmaceutical composition containing the compound and a pharmaceutically acceptable carrier. The method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject can be any mammal, e.g., a mouse, a rat, a guinea pig, a horse, a cow, a pig, a goat, a donkey, a monkey, or a human (e.g., a human patient).

In another aspect, the disclosure features an in vivo method of treating an inflammatory condition. The method includes the steps of: optionally identifying a subject, having, suspected of having, or at risk of developing, an inflammatory condition mediated by one or more immune cells expressing MUC1 and/or Abl; and delivering to the subject a compound that inhibits an interaction between MUC1 and Abl or a compound comprising a fragment of MUC1 capable of binding to and/or inhibiting Abl. The method can also include the steps of: (i) determining whether the one or more immune cells mediating the inflammatory condition express MUC1 and/or Abl and/or (ii) whether inhibition of an interaction between MUC1 and Abl or whether inhibition of Abl occurred. The subject can be a mammal such as a human. The inflammatory condition can be, e.g., rheumatoid arthritis or any other inflammatory disorder described herein. The fragment of MUC1 can consist of, or contain, a MUC1-CD (SEQ ID NO:2) or an Abl-binding fragment thereof.

In some embodiments, the method can further include administering to the subject one or more additional therapeutic agents. The one or more therapeutic agents can be a non-steroidal anti-inflammatory drug (NSAID), a disease-modifying anti-rheumatic drug (DMARD), a biological response modifier, or a corticosteroid. The biological response modifier can be an anti-TNF agent. The anti-TNF agent can be, or contain, a soluble TNF receptor or an antibody specific for TNF. The antibody specific for TNF can be, e.g., adulimumab, infliximab, or etanercept.

In some embodiments, the method can also further include, where the compound delivered to the subject is a polypeptide, administering to the subject a nucleic acid comprising a nucleotide sequence encoding the polypeptide, the nucleotide sequence being operably-linked to a transcriptional regulatory sequence. The nucleic acid can be administered in isolation, as a pharmaceutical composition containing the nucleic acid and a pharmaceutically acceptable carrier, or it can be administered in a recombinant cell transfected with the nucleic acid and secreting the polypeptide. The recombinant cell, or the progeny of the recombinant cell, can be a cell derived from another subject of the same species, or from the same subject, to be treated with the cell. The subject can be any mammal, e.g., a mouse, a rat, a guinea pig, a horse, a cow, a pig, a goat, a donkey, a monkey, or a human (e.g., a human patient).

As used herein, a subject “at risk of developing a cancer” is a subject that has a predisposition to develop a cancer, i.e., a genetic predisposition to develop cancer such as a mutation in a tumor suppressor gene (e.g., mutation in BRCA1, p53, RB, or APC), has been exposed to conditions, or is presently affected by conditions, that can result in cancer. Thus, a subject can also be one “at risk of developing a cancer” when the subject has been exposed to mutagenic or carcinogenic levels of certain compounds (e.g., carcinogenic compounds in cigarette smoke such as acrolein, 4-aminobiphenyl, aromatic amines, benzene, benz{a}anthracene, benzo{a}pyrene, formaldehyde, hydrazine, Polonium-210 (Radon), urethane, or vinyl chloride). The subject can be “at risk of developing a cancer” when the subject has been exposed to, e.g., large doses of ultraviolet light or X-irradiation, or exposed (e.g., infected) to a tumor-causing/associated virus such as papillomavirus, Epstein-Barr virus, hepatitis B virus, or human T-cell leukemia-lymphoma virus. In addition, a subject can be “at risk of developing a cancer” when the subject suffers from an inflammation (e.g., chronic inflammation) such as an inflammatory bowel disease (e.g., Crohn's disease or ulcerative colitis) or any other inflammatory condition described herein. From the above it will be clear that subjects “at risk of developing a cancer” are not all the subjects within a species of interest.

A subject “suspected of having a cancer” is one having one or more symptoms of a cancer. Symptoms of cancer are well-known to those of skill in the art and include, without limitation, breast lumps, nipple changes, breast cysts, breast pain, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreas metastases, difficulty swallowing, and the like. Types of cancers can include, e.g., lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

As used herein, a subject “at risk of developing an inflammatory condition” refers to a subject with a family history of one or more inflammatory conditions (i.e., a genetic predisposition to one or more inflammatory conditions) or one exposed to one or more inflammation-inducing conditions. For example, a subject can have been exposed to a viral or bacterial superantigen such as, but not limited to, Staphylococcal enterotoxins (SEs), a Streptococcus pyogenes exotoxin (SPE), a Staphylococcus aureus toxic shock-syndrome toxin (TSST-1), a Streptococcal mitogenic exotoxin (SME) and a Streptococcal superantigen (SSA). From the above it will be clear that subjects “at risk of developing an inflammatory condition” are not all the subjects within a species of interest.

A subject “suspected of having an inflammatory condition” is one who presents with one or more symptoms of an inflammatory condition. Symptoms of inflammatory conditions are well known in the art and include, but are not limited to, redness, swelling (e.g., swollen joints), joints that are warm to the touch, joint pain, stiffness, loss of joint function, fever, chills, fatigue, loss of energy, headaches, loss of appetite, muscle stiffness, insomnia, itchiness, stuffy nose, sneezing, coughing, one or more neurologic symptoms such as dizziness, seizures, or pain. An “inflammatory condition,” as used herein, refers to a process in which one or more substances (e.g., substances not naturally occurring in the subject), via the action of white blood cells (e.g., B cells, T cells, macrophages, monocytes, or dendritic cells) inappropriately trigger a pathological response, e.g., a pathological immune response. Accordingly, such cells involved in the inflammatory response are referred to as “inflammatory cells.” The inappropriately triggered inflammatory response can be one where no foreign substance (e.g., an antigen, a virus, a bacterium, a fungus) is present in or on the subject. The inappropriately triggered response can be one where a self-component (e.g., a self-antigen) is targeted (e.g., an autoimmune disorder such as multiple sclerosis) by the inflammatory cells. The inappropriately triggered response can also be an response that is inappropriate in magnitude or duration, e.g., anaphylaxis. Thus, the inappropriately targeted response can be due to the presence of a microbial infection (e.g., viral, bacterial, or fungal). Types of inflammatory conditions (e.g., autoimmune disease) can include, but are not limited to, osteoarthritis, rheumatoid arthritis (RA), spondyloathropathies, POEMS syndrome, inflammatory bowel diseases (e.g., Crohn's disease or ulcerative colitis), multicentric Castleman's disease, systemic lupus erythematosus (SLE), Goodpasture's syndrome, multiple sclerosis (MS), polymyalgia rheumatica, muscular dystrophy (MD), insulin-dependent diabetes mellitus (IDDM), dermatomyositis, polymyositis, inflammatory neuropathies such as Guillain Barre syndrome, vasculitis such as Wegener's granulomatosus, polyarteritis nodosa, polymyalgia rheumatica, temporal arteritis, Sjogren's syndrome, Bechet's disease, Churg-Strauss syndrome, or Takayasu's arteritis. Also included in inflammatory conditions are certain types of allergies such as rhinitis, sinusitis, urticaria, hives, angioedema, atopic dermatitis, food allergies (e.g., a nut allergy), drug allergies (e.g., penicillin), insect allergies (e.g., allergy to a bee sting), or mastocytosis. Inflammatory conditions can also include, e.g., asthma.

As used herein, a subject “at risk of developing a neurologic disorder” is a subject that (i) has a predisposition to develop a neurologic disorder (e.g., a family history of neurologic disorders or a genetic predisposition to develop a neurologic disorder such as a mutation in the GAB2 gene or the gene that encodes apolipoprotein E), (ii) is elderly, (iii) has poor cardiovascular health (e.g., the subject smokes cigarettes or has diabetes, hypertension, high cholesterol, or has had a stroke), (iv) has been exposed to certain toxins (e.g., aluminum or other trace metals), (v) has suffered a head injury, or (vi) has been exposed to certain forms of hormone replacement therapy. From the above it will be clear that subjects “at risk of developing a neurologic disorder” are not all the subjects within a species of interest.

A subject “suspected of having a neurologic disorder” is one having one or more symptoms of a neurologic disorder. Symptoms of neurologic disorders can vary depending on the nature of a specific neurologic disorder, but can include, without limitation, mild to severe cognitive impairment, incontinence, language disorders (slurring of speech), confusion, impaired gait, difficulty or inability to swallow, dementia, vertigo, severe and/or persistent headaches, difficulty moving the eyes, insomnia, ptosis, contracture of the facial muscles, or stiffening of the neck muscles. Types of neurologic disorders include, without limitation, Parkinson's disease, Alzheimer's Disease or any other tauopathy (e.g., Pick's disease, progressive supranuclear palsy (PSP), frontotemporal dementia (FTD), or corticobasal degeneration (CBD)).

As used herein, a subject “at risk of developing an diabetes-associated atherosclerotic lesion” is, generally, a subject that has diabetes or a predisposition to develop diabetes (e.g., a subject with a family history or genetic predisposition for diabetes or who is obese) or who has one or more symptoms associated with diabetes and that can lead to atherosclerosis (e.g., dyslipidemia, hypertension, coagulopathy, altered endothelium, and/or increased levels of reactive oxygen species).

A subject “suspected of having a diabetes-associated atherosclerotic lesion” is one having one or more symptoms associated with such a lesion, which can include, e.g., angina, myocardial infarction, transient or severe ischemic episodes (e.g., strokes), or pain in the legs.

“Polypeptide” and “protein” are used interchangeably and mean any peptide-linked chain of amino acids, regardless of length or post-translational modification. The MUC1, Abl molecules and test agents used in any of the methods described herein can contain or be wild-type proteins or can be variants that have not more than 50 (e.g., not more than one, two, three, four, five, six, seven, eight, nine, ten, 12, 15, 20, 25, 30, 35, 40, or 50) conservative amino acid substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. All that is required as that: (i) such variants of MUC1 have at least 25% (e.g., at least: 30%; 40%; 50%; 60%; 70%; 75%; 80%; 85%; 90%; 95%; 97%; 98%; 99%; 99.5%, or 100% or even greater) of the ability of wild-type, full-length, mature MUC1 or MUC1-CD (cytoplasmic domain) to bind to Abl (e.g., c-Abl, v-Abl, or BCR-Abl); and (ii) such variants of Abl have at least 25% (e.g., at least: 30%; 40%; 50%; 60%; 70%; 75%; 80%; 85%; 90%; 95%; 97%; 98%; 99%; 99.5%, or 100% or even greater) of the ability of the relevant wild-type, full-length Abl to bind to wild-type, full-length, mature MUC1 or MUC1-CD.

A “polypeptide fragment,” as used herein, refers to a segment of the polypeptide that is shorter than a full-length, immature polypeptide. A “functional fragment” of a polypeptide has at least 10% (e.g., at least 10%, at least 15%, at least 20%, at least 25%, at least 30%, at least 35%, at least 40%, at least 50%, at least 55%, at least 60%, at least 70%, at least 80%, at least 90%, at least 95%, at least 98%, at least 99%, at least 99.5%, or 100% or more) of the activity of the mature, polypeptide. Fragments of a polypeptide include terminal as well internal deletion variants of a polypeptide. Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids.

As used herein, an “Abl test agent” or “Abl polypeptide” contains, or is: (a) a full-length, wild-type c-Abl; (b) a drug or multidrug-resistant variant of c-Abl; (c) BCR-Abl; (d) a functional fragment of (a), (b), or (c); or (e) (a), (b), (c), or (d) with not more than 50 (see above) conservative substitutions. As above, an Abl can be c-Abl, v-Abl, BCR-Abl, or any of numerous drug and multi-drug resistant forms of Abl (Litzow, M. R. (2006) Arch Pathol Lab Med 130:669-679). The Abl polypeptides can be from any species (e.g., nematode, insect, plant, bird, reptile, or mammal (e.g., a mouse, rat, dog, cat, goat, pig, cow, horse, whale, or monkey) that expresses a homolog of a human Abl protein. “MUC1-binding fragments” of Abl polypeptides, as used herein, refer to any Abl fragments that substantially retain the ability to bind MUC1 (i.e., that have at least 25% (e.g., at least 25%, at least 30%, at least 35%, at least 40%, at least 50%, at least 55%, at least 60%, at least 70%, at least 80%, at least 90%, at least 95%, at least 98%, at least 99%, at least 99.5%, or 100% or more) of the ability of the full-length, wild-type c-Abl to bind to the cytoplasmic domain of MUC1). “Functional fragments” of Abl include fragments that contain a functional kinase domain of an Abl polypeptide.

“Abl test agents” can include internal or terminal (C or N) irrelevant or heterologous amino acid sequences (e.g., sequences derived from other proteins or synthetic sequences not corresponding to any naturally occurring protein). The sequences can be, for example, an antigenic tag (e.g., FLAG, polyhistidine, hemagluttanin (HA), glutathione-S-transferase (GST), or maltose-binding protein (MBP)). Heterologous sequences can also be proteins useful as diagnostic or detectable markers, for example, luciferase, green fluorescent protein (GFP), or chloramphenicol acetyl transferase (CAT). Heterologous sequences can be of varying length and in some cases can be a larger sequences than the Abl polypeptide. Generally, the heterologous sequences are about 1-50 (e.g., two, four, eight, ten, 15, 20, 25, 30, 35, 40, or 45) amino acids in length. Abl test agents, other than full-length, wild-type c-Abl molecules, have at least 30% (e.g., at least 30%, at least 35%, at least 40%, at least 50%, at least 55%, at least 60%, at least 70%, at least 80%, at least 90%, at least 95%, at least 98%, at least 99%, at least 99.5%, or 100% or more) of the ability of the full-length, wild-type c-Abl to bind to the cytoplasmic domain of MUC1.

As used herein, a “MUC1 test agent” or “MUC1 polypeptide” contains, or is: (a) full-length, wild-type mature MUC1; (b) a functional fragment of MUC1; or (c) (a) or (b) but with not more than 50 (see above) conservative substitutions. “MUC1 test agents” or “MUC1 polypeptides” include internal or terminal (C or N) irrelevant amino acid sequences (e.g., sequences derived from other proteins or synthetic sequences not corresponding to any naturally occurring protein) as described above for Abl test agents and Abl polypeptides. As used herein, “MUC1 cytoplasmic domain” or MUC1-CD” refers to a 72 amino acid portion of the full-length MUC1 (SEQ ID NO: 1) and is depicted in SEQ ID NO:2.

An exemplary amino acid sequence for a full-length, wild-type, mature human MUC1 polypeptide is as follows:

(SEQ ID NO:1)

MTPGTQSPFFLLLLLTVLTVVTGSGHASSTPGGEKETSATQRSSVPSSTE
KNAIPAPTTTKSCRETFLKCFCRFINKGVFWASPILSSVSDVPFPFSAQS
GAGVPGWGIALLVLVCVLVALAIYYLIALAVCQCRRKNYGQLDIFPARDT
YHPMSEYPTYHTHGRYVPPSSTDRSPYEKVSAGNGGSSLSYTNPAVAATS
ANL.

An exemplary amino acid sequence for a human MUC1-CD polypeptide is as follows:

(SEQ ID NO:2)

CQCRRKNYGQLDIFPARDTYHPMSEYPTYHTHGRYVPPSSTDRSPYEKVS
AGNGGSSLSYTNPAVAATSANL.

An exemplary amino acid sequence for a full-length, human Abl polypeptide is as follows:

(SEQ ID NO:4)

MLEICLKLVGCKSKKGLSSSSSCYLEEALQRPVASDFEPQGLSEAARWNS
KENLLAGPSENDPNLFVALYDFVASGDNTLSITKGEKLRVLGYNHNGEWC
EAQTKNGQGWVPSNYITPVNSLEKHSWYHGPVSRNAAEYLLSSGINGSFL
VRESESSPGQRSISLRYEGRVYHYRINTASDGKLYVSSESRFNTLAELVH
HHSTVADGLITTLHYPAPKRNKPTVYGVSPNYDKWEMERTDITMKHKLGG
GQYGEVYEGVWKKYSLTVAVKTLKEDTMEVEEFLKEAAVMKEIKHPNLVQ
LLGVCTREPPFYIITEFMTYGNLLDYLRECNRQEVNAVVLLYMATQISSA
MEYLEKKNFIHRDLAARNCLVGENHLVKVADFGLSRLMTGDTYTAHAGAK
FPIKWTAPESLAYNKFSIKSDVWAFGVLLWEIATYGMSPYPGIDLSQVYE
LLEKDYRMERPEGCPEKVYELMRACWQWNPSDRPSFAEIHQAFETMFQES
SISDEVEKELGKQGVRGAVSTLLQAPELPTKTRTSRRAAEHRDTTDVPEM
PHSKGQGESDPLDHEPAVSPLLPRKERGPPEGGLNEDERLLPKDKKTNLF
SALIKKKKKTAPTPPKRSSSFREMDGQPERRGAGEEEGRDISNGALAFTP
LDTADPAKSPKPSNGAGVPNGALRESGGSGFRSPHLWKKSSTLTSSRLAT
GEEEGGGSSSKRFLRSCSASCVPHGAKDTEWRSVTLPRDLQSTGRQFDSS
TFGGHKSEKPALPRKRAGENRSDQVTRGTVTPPPRLVKKNEEAADEVFKD
IMESSPGSSPPNLTPKPLRRQVTVAPASGLPHKEEAGKGSALGTPAAAEP
VTPTSKAGSGAPGGTSKGPAEESRVRRHKHSSESPGRDKGKLSRLKPAPP
PPPAASAGKAGGKPSQSPSQEAAGEAVLGAKTKATSLVDAVNSDAAKPSQ
PGEGLKKPVLPATPKYQSAKPSGTPISPAPVPSTLPSASSALAGDQPSST
AFIPLISTRVSLRKTRQPPERIASGAITKGVVLDSTEALCLAISRNSEQM
ASHSAVLEAGKNLYTFCVSYVDSIQQMRNKFAFREAINKLENNLRELQIC
PATAGSGPAATQDFSKLLSSVKEISDUVQR.

An exemplary amino acid sequence for an SH2 domain fragment of a human Abl polypeptide (e.g., amino acids 127-217 of SEQ ID NO:4) is as follows:

(SEQ ID NO:5)

WYHGPVSRNAAEYLLSSGINGSFLVRESESSPGQRSISLRYEGRVYHYRI
NTASDGKLYVSSESRFNTLAELVHHHSTVADGLITTLHYPA.

An exemplary amino acid sequence for an SH3 domain fragment of a human Abl polypeptide (e.g., amino acids 65-126 of SEQ ID NO:4) is as follows:

(SEQ ID NO:8)

NLFVALYDFVASGDNTLSITKGEKLRVLGYNHNGEWCEAQTKNGQGWVPS
NYITPVNSLEKHS.

Depending on their intended use, the polypeptides, test agents, or fragments of the polypeptides or test agents described herein can be of any species such as, e.g., nematode, insect, plant, bird, fish, reptile, or mammal (e.g., a mouse, rat, rabbit, hamster, gerbil, dog, cat, goat, pig, cow, horse, whale, monkey, or human). In some embodiments, fragments can include immunogenic and antigenic fragments of the polypeptides or test agents. An immunogenic fragment is one that has at least 25% (e.g., at least: 30%; 40%; 50%; 60%; 70%; 75%; 80%; 85%; 90%; 95%; 97%; 98%; 99%; 99.5%, or 100% or even more) of the ability of the relevant full-length, wild-type protein to stimulate an immune response (e.g., an antibody response or a cellular immune response) in an animal of interest. An antigenic fragment of a protein is one having at least 25% (e.g., at least: 30%; 40%; 50%; 60%; 70%; 75%; 80%; 85%; 90%; 95%; 97%; 98%; 99%; 99.5%, or 100% or even greater) of the ability of the relevant full-length, wild-type polypeptide or test agent to be recognized by an antibody specific for the protein or a T cell specific to the protein.

As used herein, “operably linked” means incorporated into a genetic construct so that expression control sequences effectively control expression of a coding sequence of interest.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications (including U.S. application Ser. No. 10/733,212, U.S. Provisional Application No. 60/710,166, and International Application Serial No. PCT/US2006/0052), patents, and other references mentioned herein are incorporated by reference in their entirety. The materials, methods, and examples disclosed herein are illustrative only and not intended to be limiting.

Other features and advantages of the invention, e.g., inhibiting survival of cancer cells, will be apparent from the following description, from the drawings and from the claims.

### DESCRIPTION OF THE DRAWINGS

FIG. 1A is a series of photographs of immunoblots. Lysates from HeLa cells were subjected to immunoblotting using anti-MUC1-N, anti-MUC1-C, anti-c-Abl or anti-β-actin antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 1B and 1C are a series of photographs of immunoblots. HeLa/vector and HeLa/MUC1 cells were treated either with 1 μM adriamycin (ADR) (FIG. 1B) or 100 μM cisplatin (CDDP) (FIG. 1C) for 2 hours. Nuclear fractions were prepared from the indicated cells and subjected to immunoblot analysis using antibodies specific for c-Abl, lamin B or IκBα. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 1D is a pair of photographs of immunoblots. HeLa/vector and HeLa/MUC1 cells were treated with CDDP for 2 hours. Cytosolic extracts were prepared from the cells and subjected to immunoblot analysis using anti-c-Abl or anti-β-actin antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 2A is a series of photographs of immunoblots. Whole-cell lysates from ZR-75-1/vector and ZR-75-1/MUC1siRNA cells were subjected to immunoblotting using anti-MUC1-N, anti-MUC1-C, anti-c-Abl or anti-β-actin antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 2B and 2C are two series of photographs of immunoblots. ZR-75-1/vector and ZR-75-1/MUC1siRNA cells were treated with CDDP for 2 and 4 hours. Nuclear (FIG. 2B) and cytosolic (FIG. 2C) fractions of the CDDP-treated cells were subjected to immunoblot analysis using the specific antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 3A is a series of photographs of immunoblots. 293 cells were transiently transfected with GFP-c-Abl (fusion protein of Green Fluorescent Protein (GFP) and c-Abl) in the absence and presence of co-expressed MUC1. After 48 hours, the cells were treated with ADR for 2 hours. Nuclear fractions or whole cell lysates (WCL) were immunoblotted using the antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 3B is a series of photographs of immunoblots. 293 cells were transiently transfected with GFP-c-Abl (fusion protein of Green Fluorescent Protein and c-Abl) or GFP-c-AblΔNES (fusion protein of Green Fluorescent Protein and a mutant c-Abl polypeptide wherein the nuclear export signal (NES) has been deleted) in the absence and presence of co-expressed MUC1. After 48 hours, the cells were treated with ADR for 2 hours. Nuclear fractions or whole cell lysates (WCL) were immunoblotted using the antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 4A and 4B are both pairs of photographs of immunoblots. WCLs from HeLa/MUC1 (FIG. 4A) and ZR-75-1/vector (FIG. 4B) cells were subjected to immunoprecipitation (IP) with mouse IgG (control) or antibodies specific for MUC1-N. The precipitates were subjected to sodium dodecyl sulfate-polyacrylamide gel electrophoresis (SDS-PAGE) and immunoblotted with anti-c-Abl or anti-MUC1-N antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 4C is a series of photographs of immunoblots. Cell membrane fractions from HeLa/vector and HeLa/MUC1 cells were subjected to immunoblot analysis using the antibodies indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 4D is a series of photographs of immunoblots and a bar graph. Cell membrane fractions from ZR-75-1/vector or ZR-75-1/MUC1siRNA cells were subjected to immunoblot analysis using the specific antibodies indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs. Immunoblots from three separate experiments were subjected to densitometric scanning to determine the effects of MUC silencing on localization of c-Abl to the cell membrane. The bar graph at the bottom of the series of photographs shows the results of these determinations, and the data in the graph are expressed as the percentage (mean ±SD) of c-Abl localization for ZR-75-1/vector cells (assigned a value of 100%) and ZR-75-1/MUC1siRNA cells.

FIG. 4E is a series of photographs of fluorescently labeled cells. Confocal microscopy of ZR-75/vector cells stained with an anti-MUC1-C antibody conjugated to Fluorescein isothiocyanate (FITC) (green) or anti-c-Abl conjugated to Texas Red (red). Nuclei were stained with TO-PRO-3 (blue). Merged images of MUC1-C and c-Abl are indicated in yellow.

FIG. 5 is a pair of photographs of immunoblots. HCT116 cells were treated with CDDP for 1 hour. Lysates were prepared from the CDDP-treated cells and c-Abl was immunoprecipitated using anti-c-Abl antibodies. c-Abl immunoprecipitates were incubated with Crk(120-225) and [γ-³²P]ATP. The reaction products were analyzed by SDS-PAGE and autoradiography. The anti-c-Abl precipitates were also immunoblotted with anti-c-Abl as a control. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 6A is a series of photographs of immunoblots. WCLs from HCT116/vector and HCT116/Flag-MUC1-CD cells were subjected to SDS-PAGE and immunoblotted using anti-MUC1-C, anti-c-Abl or anti-β-actin antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 6B is a pair of photographs of immunoblots. WCLs were prepared from HCT116/Flag-MUC1-CD cells and were subsequently immunoprecipitated with either mouse IgG or anti-Flag antibodies. The immunoprecipitates were subjected to SDS-PAGE and immunoblotted using antibodies specific for c-Abl or Flag as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 6C is a pair of photographs of immunoblots. Purified GST-MUC1-CD (a fusion protein of glutathione-S-transferase fused in frame with MUC1-cytoplasmic domain (MUC1-CD)) was cleaved with thrombin to remove the GST tag and then incubated with GST-c-Abl (a fusion protein of glutathione-S-transferase fused in frame with a wild-type c-Abl polypeptide) or GST-c-Abl(K-R) (a fusion protein of glutathione-S-transferase fused in frame with a kinase defective mutant c-Abl polypeptide (K-R)) and [γ-³²P]ATP for 15 minutes at 30° C. The reaction products were analyzed by SDS-PAGE and autoradiography (top panel) or by immunoblotting with anti-MUC1-C (lower panel). “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 6D is a depiction of the amino acid sequence of MUC1-CD (SEQ ID NO:2) and a pair of photographs of immunoblots. Purified MUC1-CD or MUC1-CD(Y60F) was incubated with GST-c-Abl or GST-c-Abl(K-R) and [γ-³²P]ATP for 15 min at 30° C. (as indicated in the chart above the photograph). Reaction products were analyzed by SDS-PAGE and autoradiography or by immunoblotting with anti-MUC1-C (lower panels). “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 7A is a series of photographs of immunoblots. WCLs from HCT116/MUC1 (clones A and B) and HCT116/MUC1(Y60F) (clones A and B) were subjected to SDS-PAGE followed by immunoblotting using antibodies specific for MUC1-N, MUC1-C, c-Abl or β-actin as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 7B is a series of photographs of immunoblots. WCLs from HCT116/MUC-1 and HCT116/MUC1(Y60F) cells were immunoprecipitated with mouse IgG and anti-MUC1-N. The precipitates were subjected to SDS-PAGE followed by immunoblotting using anti-c-Abl, anti-MUC1-N and anti-MUC1-C. “113,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 7C is a series of photographs of immunoblots. Cell membrane fractions from HCT116/vector, HCT116/MUC1 and HCT116/MUC1(Y60F) cells were prepared, subjected to SDS-PAGE, and immunoblotted using the antibodies indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 7D is a pair of photographs of immunoblots. 293T cells were transiently transfected with an expression vector containing GFP (Green Fluorescent Protein) and MUC1, GFP-c-Abl and MUC1, and GFP-c-Abl and MUC1(Y60F) coding sequences. Lysates were prepared from the above-described transiently transfected 293T cells and subjected to immunoprecipitation with anti-MUC1-N antibodies or control IgG. Immunoprecipitates were subjected to SDS-PAGE followed by immunoblotting of the precipitates using anti-GFP and anti-MUC1-N antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 7E is a pair of photographs of immunoblots. Purified MUC1-CD and MUC1-CD (Y60F) were incubated in presence or absence of recombinant, truncated Abl (no SH2 domain) and 200 μM ATP for 30 minutes at 30° C. GST or GST-c-Abl-SH2 bound to GST-beads was then added and incubated for 1 hour at 4° C. Adsorbed (to the beads) (upper panel) and input (lower panel) proteins were subjected to SDS-PAGE and immunoblot analysis using anti-MUC1-C antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 8A is a series of photographs of immunoblots and a Coomassie blue-stained electrophoretic gel. Purified MUC1-CD was incubated with recombinant truncated Abl (no SH2 domain) and 200 μM ATP for 30 minutes at 30° C. GST, GST-c-Abl-SH2 or GST-c-Abl-SH2(R152L) (a mutant c-Abl protein where the arginine at position 152 was changed to a leucine) bound to glutathione beads was then added for 1 hour at 4° C. Adsorbed (upper panel) and input (middle panel) proteins were subjected SDS-PAGE and immunoblot analysis using antibodies specific for MUC1-C. “IB,” where used below a photograph, refers to “immunoblot” and is followed by the antibody used for immunoblotting in the above photograph. Equal loading of the various GST proteins was assessed by Coomassie blue staining of the SDS-PAGE gel [bottom panel]. The molecular weights of the proteins (expressed in kilodaltons (kDa)) are indicated at the left of each of the photographs.

FIGS. 8B, 8C, and 8D are each a series of photographs of immunoblots. 293T cells were transiently transfected with expression vectors containing MUC1 and GFP-c-Abl or GFP-c-Abl(R152L) coding sequences as indicated. At 48 hours after transfection, the cells were left untreated or treated with ADR for 2 hours. WCLs (FIG. 8B), cytoplasmic fractions (FIG. 8C) and nuclear fractions (FIG. 8D) were immunoblotted using the specific antibodies indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 9A, 9B, and 9C are each pairs of photographs of immunoblots. WCLs were prepared from: (FIG. 9A) HeLa/vector and HeLa/MUC1 cells; (FIG. 9B) ZR-75-1/vector, ZR-75-1/MUC1siRNA cells; or (FIG. 9C) HCT116/vector, HCT116/MUC1 and HCT116/MUC1(Y60F) cells. Each of the lysates was immunoprecipitated using an anti-c-Abl antibody. The resulting immunoprecipitates were subjected to SDS-PAGE and immunoblotted with anti-phospho-c-Abl-Thr-735 and anti-c-Abl antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 9D, 9E, and 9F are each pairs of photographs of immunoblots. Cytoplasmic fractions of (FIG. 9D): HeLa/vector and HeLa/MUC1 cells; (FIG. 9E) ZR-75-1/vector and ZR-75-1/MUC1siRNA cells; or (FIG. 9F) HCT116/vector, HCT116/MUC1 and HCT116/MUC1(Y60F) (FIG. 9F) cells were prepared. Fractions were immunoprecipitated using antibodies specific for 14-3-3. The precipitates were subjected to SDS-PAGE followed by immunoblotting using anti-c-Abl and anti-14-3-3 antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 10A is a series of photographs of immunoblots. HCT116/vector, HCT116/MUC1 and HCT116/MUC1(Y60F) cells were treated with CDDP for 2 and 4 hours. Nuclear lysates were then prepared from the CDDP treated cells and subjected to SDS-PAGE followed by immunoblotting with anti-c-Abl, anti-lamin B, and anti-IκBα antibodies. “IB,” where used below a photograph, refers to “immunoblot” and is followed by the antibody used for immunoblotting in the above photograph. Intensity of the signals was determined by densitometric scanning and compared to that for untreated cells. Numerical values determined from the scanning are given as “fold” expression over untreated cells and indicated below the upper photograph. The molecular weights of the proteins (expressed in kilodaltons (kDa)) are indicated at the left of each of the photographs.

FIG. 10B is a pair of photographs of immunoblots. Anti-MUC1-N immunoprecipitates from HCT116/MUC1 cell lysates, prepared from cells treated with CDDP for 1, 2, and 4 hours, were subjected to immunoblotting with anti-c-Abl and anti-MUC1-N antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 10C is a pair of photographs of immunoblots. HCT116/vector, HCT116/MUC and HCT116/MUC1(Y60F) cells were treated with CDDP for the indicated times. WCLs were prepared from the cells and subjected to SDS-PAGE and immunoblotting using anti-PKCδ (Protein Kinase Cδ) and anti-β-actin antibodies. “FL” indicates full length PK-Cδ, and “CF” indicates cleaved fragments of PKCδ. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 10D is a bar graph showing the results from sub-G1 DNA Fluorescence-Activated Cell Sorting (FACS) analysis of HCT116/vector, HCT116/MUC1 and HCT116/MUC1(Y60F) cells treated with CDDP for 0 h (open bars), 12 h (solid bars) or 24 h (hatched bars). The data are expressed as the percentage of apoptosis over control (mean ±S.D. of three separate experiments).

FIG. 10E is a bar graph showing the results from sub-G1 DNA analysis of HCT116/vector, HCT116/MUC1 and HCT116/MUC1(Y60F) cells left untreated (open bars) or treated with CDDP alone for 12 h (solid bars), STI571 alone for 12 h (shaded bars) or both CDDP and STI571 for 12 h (hatched bars). The data are expressed as the percentage of apoptosis over untreated control (mean ±S.D. of three separate experiments).

FIG. 10F is a series of bar graphs showing the results from sub-G1 DNA analysis of HCT116/vector [left panel], HCT116/MUC1 [center panel] and HCT116/MUC1(Y60F) [right panel] cells transfected with the control CsiRNA or c-AblsiRNA and left untreated (open bars) or treated with CDDP for 12 h (solid bars). The data are expressed as the percentage of apoptosis over untreated controls (mean ±S.D. of three separate experiments). “N.S.,” where used in relation to a p-value, refers to “Not Significant.”

FIG. 11 is a series of photographs of immunoblots. HCT116/MUC1 cells were treated with CDDP for 0, 1, 2, and 4 hours. Nuclear fractions or WCLs were prepared from the treated cells and subjected to SDS-PAGE and immunoblotting using the antibodies indicated. “IB,” where used below a photograph, refers to “immunoblot” and is followed by the antibody used for immunoblotting in the above photograph. The intensity of the signals was determined by densitometric scanning and compared to that for untreated cells. Numeric values as determined from the scanning are given as “fold” expression over untreated cells and indicated below the photographs. The molecular weights of the proteins (expressed in kilodaltons (kDa)) are indicated at the left of each of the photographs.

FIG. 12 is a series of FACS analysis profiles depicting the attenuation of MUC1-associated anti-apoptotic effects by the Y60F mutation of MUC1. HCT116/vector, HCT116/MUC1 and HCT116/MUC1(Y60F) cells were treated with CDDP for 12 hours and analyzed for sub-G1 DNA using propidium iodide staining and FACS analysis.

FIG. 13A is a series of photographs of immunoblots. HCT116/vector [left panel], HCT116/MUC1 [center panel] and HCT116/MUC1(Y60F) [right panel] cells were transiently transfected with a control siRNA (CsiRNA) or a pool of c-Abl siRNAs. At 72 h after transfection, lysates were prepared from the transfected cells and subjected to SDS-PAGE and immunoblotting using specific antibodies as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 13B, 13C, and 13D are a series of FACS analysis profiles. (FIG. 13B) HCT116/vector; (FIG. 13C) HCT116/MUC1; and (FIG. 13D) HCT116/MUC1(Y60F) stable cells were left untreated (Control), or treated with c-Abl siRNA as indicated. These cell populations were then treated with CDDP for 12 hours and all analyzed for sub-G1 DNA content.

FIG. 14A is a series of photographs of immunoblots. WCLs were prepared from HCT116/MUC1(Y60F) and HCT116/MUC1(Y46F) cells. Lysates were subjected to SDS-PAGE followed by immunoblotting using antibodies specific for MUC1-N, MUC1-C, c-Abl, or β-actin as indicated. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 14B is a pair of photographs of immunoblots. Anti-MUC1-N immunoprecipitates from lysates prepared from: HCT116/MUC1, HCT116 MUC1(Y46F) and HCT116/MUC1(Y60F) cells, were subjected to SDS-PAGE and immunoblotting using anti-c-Abl and anti-MUC1-N antibodies. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIGS. 14C and 14D are two series of photographs of immunoblots. HCT116/MUC1(Y60F) and HCT116/MUC1(Y46F) stably transfected cells were treated with CDDP for 0, 2, or 4 hours. Nuclear (FIG. 14C) and cytoplasmic (FIG. 14D) fractions were prepared from each of the treated cell populations, subjected to SDS-PAGE and immunoblotting using antibodies specific for c-Abl or lamin-B as a control. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 14E is a series of photographs of immunoblots. Mitochondrial fractions from HCT116/MUC1(Y60F) and HCT116/MUC1(Y46F) cells were prepared. Fractions were subjected to SDS-PAGE followed by immunoblotting with antibodies specific for MUC1-C. Immunoblotting was also performed with antibodies to HSP60, or PCNA, Calnexin, and IκBα (lower series of photographs) as controls for both loading and purity of the mitochondrial fractionation. “IB,” where used below a photograph, refers to “immunoblot” and is followed by the antibody used for immunoblotting in the above photograph. The molecular weights of the proteins (expressed in kilodaltons (kDa)) are indicated at the left of each of the photographs.

FIG. 15 is a depiction of an exemplary amino acid sequence for a human c-Abl protein (NCBI Accession No. P00519) (SEQ ID NO:4).

### DETAILED DESCRIPTION

Methods of Screening for Inhibitory Compounds

The disclosure provides in vitro methods (e.g, “screening methods”) for identifying compounds (e.g., small molecules or macromolecules) that inhibit binding of Abl (e.g., c-Abl or functional fragment thereof) to MUC1.

These methods can be performed using: (a) isolated MUC1 test agents and one or more isolated Abl test agents; or (b) cells expressing a MUC1 test agent and one or more Abl test agents.

The term “isolated” as applied to any of the above-listed polypeptide test agents refers to a polypeptide, or a peptide fragment thereof, which either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue (e.g., breast cancer or colon cancer tissue), or body fluids such as blood, serum, or urine. Typically, the polypeptide or peptide fragment is considered “isolated” when it is at least 70%, by dry weight, free from the proteins and other naturally-occurring organic molecules with which it is naturally associated. Preferably, a preparation of a test agent is at least 80%, more preferably at least 90%, and most preferably at least 99%, by dry weight, the test agent. Since a polypeptide that is chemically synthesized is, by its nature, separated from the components that naturally accompany it, a synthetic polypeptide test agent is “isolated.”

An isolated polypeptide test agent can be obtained, for example, by extraction from a natural source (e.g., from tissues); by expression of a recombinant nucleic acid encoding the polypeptide; or by chemical synthesis. A polypeptide test agent that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

Prior to testing, any of the test agents can undergo modification, e.g., phosphorylation or glycosylation, by methods known in the art.

In methods of screening for compounds that inhibit or enhance binding of an isolated MUC1 test agent to an isolated Abl test agent, a MUC1 test agent is contacted with an Abl test agent in the presence of one or more concentrations of a test compound and binding between the two test agents in the presence and absence of the test compound is detected, tested for, and/or measured. In such assays neither of the test agents need be detectably labeled. For example, by exploiting the phenomenon of surface plasmon resonance, the MUC1 test agent can be bound to a suitable solid substrate and an Abl test agent exposed to the substrate-bound MUC1 test agent in the presence and absence of the compound of interest. Binding of the Abl test agent to the MUC1 test agent on the solid substrate results in a change in the intensity of surface plasmon resonance that can be detected qualitatively or quantitatively by an appropriate instrument, e.g., a Biacore apparatus (Biacore International AB, Rapsgatan, Sweden). It will be appreciated that the experiment can be performed in reverse, i.e., with the Abl test agent bound to the solid substrate and the MUC1 test agent added to it in the presence of the test compound.

Moreover, assays to test for inhibition (or in some cases enhancement) of binding to MUC1 can involve the use, for example, of: (a) a single MUC1-specific “detection” antibody that is detectably labeled; (b) an unlabeled MUC1-specific antibody and a detectably labeled secondary antibody; or (c) a biotinylated MUC1-specific antibody and detectably labeled avidin. In addition, combinations of these approaches (including “multi-layer” assays) familiar to those in the art can be used to enhance the sensitivity of assays. In these assays, the Abl test agent can be immobilized on a solid substrate such as a nylon or nitrocellulose membrane by, for example, “spotting” an aliquot of a sample containing the test agent onto a membrane or by blotting onto a membrane an electrophoretic gel on which the sample or an aliquot of the sample has been subjected to electrophoretic separation. Alternatively, the Abl test agent can be bound to a plastic substrate (e.g., the plastic bottom of an ELISA (enzyme-linked immunosorbent assay) plate well) using methods known in the art. The substrate-bound test agent is then exposed to the MUC1 test agent in the presence and absence of the test compound. After incubating the resulting mixture for a period of time and at temperature optimized for the system of interest, the presence and/or amount of MUC1 test agent bound to the Abl test on the solid substrate is then assayed using a detection antibody that binds to the MUC1 test agent and, where required, appropriate detectably labeled secondary antibodies or avidin. It will be appreciated that instead of binding the Abl test agent to the solid substrate, the MUC1 test agent can be bound to it. In this case, binding of the Abl test agent to the substrate-bound MUC1 is tested by obvious adaptions of the method described above for substrate-bound Abl test agent.

The disclosure also features “sandwich” assays. In these sandwich assays, instead of immobilizing test agents on solid substrates by the methods described above, an appropriate test agent can be immobilized on the solid substrate by, prior to exposing the solid substrate to the test agent, conjugating a “capture” test agent-specific antibody (polyclonal or mAb) to the solid substrate by any of a variety of methods known in the art. The test agent is then bound to the solid substrate by virtue of its binding to the capture antibody conjugated to the solid substrate. The procedure is carried out in essentially the same manner described above for methods in which the appropriate test agent is bound to the solid substrate by techniques not involving the use of a capture antibody. It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either: (a) a mAb that binds to an epitope that is either completely physically separated from or partially overlaps with any of the epitopes to which the capture polyclonal antibody binds; or (b) a polyclonal antibody that binds to epitopes other than or in addition to that to which the capture polyclonal antibody binds. Assays which involve the use of a capture and a detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, polymeric (e.g., without limitation, agarose, cellulose, or polyacrylamide) beads or particles.

Methods of detecting and/or for quantifying a detectable label depend on the nature of the label and are known in the art. Appropriate labels include, without limitation, radionuclides (e.g., ¹²⁵I, ¹³¹I, ³⁵S, ³H, ³²P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive or they may absorb visible or ultraviolet light. Examples of detectors include, without limitation, x-ray film, radioactivity counters, scintillation counters, spectrophotometers, colorimeters, fluorometers, luminometers, and densitometers.

Candidate compounds can also be tested for their ability to inhibit binding of MUC to an Abl in cells. The cells can either naturally express an appropriate MUC test agent and/or an Abl test agent of interest (i.e., the cells encode an endogenous MUC1 and/or Abl gene which can be expressed to yield a MUC1 and/or Abl polypeptide) or they can recombinantly express either or both test agents. The cells can be normal or malignant and of any histological type, e.g., without limitation, epithelial cells, fibroblasts, lymphoid cells, macrophages/monocytes, granulocytes, keratinocytes, neuronal cells, or muscle cells. Suitable cell lines include those recited in the examples, e.g., breast cancer or colon cancer cell lines. The test compound can be added to the solution (e.g., culture medium) containing the cells or, where the compound is a protein, the cells can recombinantly express it. The cells can optionally also be exposed to a stimulus of interest (e.g., a growth factor such as EGF) prior to or after exposure of the cells to the compound. Following incubation of cells expressing the test agents of interest in the absence or presence (optionally at various concentrations), physical association between the test agents can be determined microscopically using appropriately labeled antibodies specific for both test agents, e.g., by confocal microscopy. Alternatively, the cells can be lysed under non-dissociating conditions and the lysates tested for the presence of physically associated test agents. Such methods include adaptations of those described using isolated test agents. For example, an antibody specific for one of the two test agents (test agent 1) can be bound to a solid substrate (e.g., the bottom and sides of the well of a microtiter plate or a nylon membrane). After washing away unbound antibody, the solid substrate with bound antibody is contacted with the cell lysate. Any test agent 1 in the lysate, bound or not bound to the second test agent (test agent 2), will bind to the antibody specific for test agent 1 on the solid substrate. After washing away unbound lysate components, the presence of test agent 2 (bound via test agent 1 and the antibody specific for test agent 1 to the solid substrate) is tested for using a detectably labeled antibody (see above) specific for test agent 2. Alternatively, test agent 1 can be immunoprecipitated with an antibody specific for test agent 1 and the immunoprecipitated material can be subjected to electrophoretic separation (e.g., by polyacrylamide gel electrophoresis performed under non-dissociating conditions). The electrophoretic gel can then be blotted onto a membrane (e.g., a nylon or a nitrocellulose membrane) and any test agent 2 on the membrane detected and/or measured with a detectably labeled antibody (see above) specific for test agent 2 by any of the above-described methods. It is understood that in the above-described assays, test agent 1 can be either the MUC1 test agent or the Abl test agent or vice versa. The test compounds can bind to one or both of the MUC1 and Abl test agents.

Methods of Designing and Producing Inhibitory Compounds

The present disclosure also relates to using MUC1 test agents and/or Abl test agents to predict or design compounds that can interact with MUC1 and/or Abl and potentially thereby inhibit the interaction between these two polypeptides. Such compounds would be useful to inhibit the ability of MUC1 to promote cell survival (through MUC1-inhibition of the localization of Abl to the cell nucleus). One of skill in the art would know how to use standard molecular modeling or other techniques to identify small molecules that would bind to “appropriate sites” on MUC1 and/or Abl. One such example is provided in Broughton (1997) Curr. Opin. Chem. Biol. 1, 392-398. Generally, an “appropriate site” on a MUC1 or Abl is a site directly involved in the physical interaction between the two molecule types. However, an “appropriate site” can also be an allosteric site, i.e., a region of the molecule not directly involved in a physical interaction with another molecule (and possibly even remote from such a “physical interaction” site) but to which binding of a compound results (e.g., by the induction in a conformational change in the molecule) in inhibition of the binding of the molecule to another molecule.

By “molecular modeling” is meant quantitative and/or qualitative analysis of the structure and function of protein-protein physical interaction based on three-dimensional structural information and protein-protein interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods.

Methods of designing compounds that bind specifically (e.g., with high affinity) to the region of MUC1 that interacts with Abl (i.e., the cytoplasmic domain of MUC1) or the region of an Abl that binds to MUC1 (i.e., the SH2 domain of Abl of SEQ ID NO:5) typically are also computer-based, and involve the use of a computer having a program capable of generating an atomic model. Computer programs that use X-ray crystallography data are particularly useful for designing such compounds. Programs such as RasMol, for example, can be used to generate a three dimensional model of, e.g., the region of MUC1 that interacts with Abl or the region of Abl that binds to MUC1 and/or determine the structures involved in MUC1-Abl binding. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures. Compounds can be designed using, for example, computer hardware or software, or a combination of both. However, designing is preferably implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object oriented programming language to communicate with a computer system. However, the programs can be implemented in assembly or machine language, if desired. In any case, the language can be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method described herein can also be implemented by means of a computer-readable storage medium, configured with a computer program, where the storage medium so configured causes a computer to operate in a specific and predefined manner to perform the functions described herein.

For example, the computer-requiring steps in a method of designing a compound can involve:

(a) inputting into an input device, e.g., through a keyboard, a diskette, or a tape, data (e.g. atomic coordinates) that define the three-dimensional (3-D) structure of a first molecule (e.g., MUC1 or a part of MUC1) that binds to a second molecule (e.g., Abl or a part thereof) or a molecular complex (e.g., MUC1, or a part thereof, bound to Abl, or a part thereof, or MUC1 bound to a macromolecular Abl complex), e.g., a region of MUC1 that interacts with Abl (i.e., the cytoplasmic domain of MUC1), the region of Abl that binds to MUC1 (i.e., the SH2 domain of c-Abl as depicted in SEQ ID NO:5), or all or a part (e.g., the cytoplasmic domain) of MUC1 bound to all or a part of Abl; and

(b) determining, using a processor, the 3-D structure (e.g., an atomic model) of: (i) the site on the first molecule involved in binding to the second molecule; or (ii) one or more sites on the molecular components of molecular complex of interaction between molecular components of the molecular complex.

From the information obtained in this way, one skilled in the art will be able to design and make inhibitory compounds (e.g., peptides, non-peptide small molecules, aptamers (e.g., nucleic acid aptamers) with the appropriate 3-D structure.

Moreover, if computer-usable 3-D data (e.g., x-ray crystallographic or nuclear magnetic resonance (NMR) data) for a candidate compound are available, the following computer-based steps can be performed in conjunction with computer-based steps (a) and (b) described above: (c) inputting into an input device, e.g., through a keyboard, a diskette, or a tape, data (e.g. atomic coordinates) that define the three-dimensional (3-D) structure of a candidate compound; (d) determining, using a processor, the 3-D structure (e.g., an atomic model) of the candidate compound; (e) determining, using the processor, whether the candidate compound binds to the site on the first molecule or the one or more sites on the molecular components of the molecular complex; and (f) identifying the candidate compound as a compound that inhibits the interaction between the first and second molecule or the between the molecular components of the molecular complex.

The method can involve the additional step of outputting to an output device a model of the 3-D structure of the compound. In addition, the 3-D data of candidate compounds can be compared to a computer database of, for example, 3-D structures (e.g., of MUC1, the cytoplasmic domain of MUC1, Abl, or a MUC1-interacting fragment of Abl) stored in a data storage system.

Compounds useful for the methods described herein also may be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917). Compounds and polypeptides can also be identified by, for example, identifying candidate compounds by computer modeling as fitting spatially and preferentially (i.e., with high affinity) into the appropriate acceptor sites on MUC1 or Abl.

Candidate compounds identified as described above can then be tested in standard cellular or cell-free binding or binding inhibition assays familiar to those skilled in the art. Exemplary assays are described herein.

A candidate compound whose presence requires at least 2-fold (e.g., 4-fold, 6-fold, 10-fold, 100-fold, 1000-fold, 10,000 fold, or 100,000-fold) more of a given MUC1 test agent to achieve a defined arbitrary level of binding to a fixed amount of an Abl test agent than is achieved in the absence of the compound can be useful for inhibiting the interaction between MUC1 and the relevant Abl, and thus can be useful as a cancer therapeutic or prophylactic agent. Alternatively, a candidate compound whose presence requires at least 2-fold (e.g., 2-fold, 4-fold, 6-fold, 10-fold, 100-fold, 1000-fold, 10,000 fold, or 100,000-fold) more of a given Abl test agent to achieve a defined arbitrary level of binding to a fixed amount of a MUC1 test agent than is achieved in the absence of the compound can be useful for inhibiting the interaction between MUC1 and the relevant Abl, and thus can be useful as a cancer therapeutic or prophylactic agent.

The 3-D structure of biological macromolecules (e.g., proteins, nucleic acids, carbohydrates, and lipids) can be determined from data obtained by a variety of methodologies. These methodologies, which have been applied most effectively to the assessment of the 3-D structure of proteins, include: (a) x-ray crystallography; (b) nuclear magnetic resonance (NMR) spectroscopy; (c) analysis of physical distance constraints formed between defined sites on a macromolecule, e.g., intramolecular chemical crosslinks between residues on a protein (e.g., International Patent Application No. PCT/US00/14667, the disclosure of which is incorporated herein by reference in its entirety), and (d) molecular modeling methods based on a knowledge of the primary structure of a protein of interest, e.g., homology modeling techniques, threading algorithms, or ab initio structure modeling using computer programs such as MONSSTER (Modeling Of New Structures from Secondary and Tertiary Restraints) (see, e.g., International Application No. PCT/US99/11913, the disclosure of which is incorporated herein by reference in its entirety). Other molecular modeling techniques may also be employed in accordance with the methods described herein[e.g., Cohen et al. (1990) J. Med. Chem. 33: 883-894; Navia et al (1992) Current Opinions in Structural Biology, 2, pp. 202-210, the disclosures of which are incorporated herein by reference in its entirety]. All these methods produce data that are amenable to computer analysis. Other spectroscopic methods that can also be useful in the methods described herein, but that do not currently provide atomic level structural detail about biomolecules, include circular dichroism and fluorescence and ultraviolet/visible light absorbance spectroscopy. A preferred method of analysis is x-ray crystallography. Descriptions of this procedure and of NMR spectroscopy are provided below.

X-Ray Crystallography

X-ray crystallography is based on the diffraction of x-radiation of a characteristic wavelength by electron clouds surrounding the atomic nuclei in a crystal of a molecule or molecular complex of interest. The technique uses crystals of purified biological macromolecules or molecular complexes (but these frequently include solvent components, co-factors, substrates, or other ligands) to determine near atomic resolution of the atoms making up the particular biological macromolecule. A prerequisite for solving 3-D structure by x-ray crystallography is a well-ordered crystal that will diffract x-rays strongly. The method directs a beam of x-rays onto a regular, repeating array of many identical molecules so that the x-rays are diffracted from the array in a pattern from which the structure of an individual molecule can be retrieved. Well-ordered crystals of, for example, globular protein molecules are large, spherical or ellipsoidal objects with irregular surfaces. The crystals contain large channels between the individual molecules. These channels, which normally occupy more than one half the volume of the crystal, are filled with disordered solvent molecules, and the protein molecules are in contact with each other at only a few small regions. This is one reason why structures of proteins in crystals are generally the same as those of proteins in solution.

Methods of obtaining the proteins of interest are described below. The formation of crystals is dependent on a number of different parameters, including pH, temperature, the concentration of the biological macromolecule, the nature of the solvent and precipitant, as well as the presence of added ions or ligands of the protein. Many routine crystallization experiments may be needed to screen all these parameters for the combinations that give a crystal suitable for x-ray diffraction analysis. Crystallization robots can automate and speed up work of reproducibly setting up a large number of crystallization experiments (see, e.g., U.S. Pat. No. 5,790,421, the disclosure of which is incorporated herein by reference in its entirety). Polypeptide crystallization occurs in solutions in which the polypeptide concentration exceeds it's solubility maximum (i.e., the polypeptide solution is supersaturated). Such solutions may be restored to equilibrium by reducing the polypeptide concentration, preferably through precipitation of the polypeptide crystals. Often polypeptides may be induced to crystallize from supersaturated solutions by adding agents that alter the polypeptide surface charges or perturb the interaction between the polypeptide and bulk water to promote associations that lead to crystallization.

Crystallizations are generally carried out between 4° C. and 20° C. Substances known as “precipitants” are often used to decrease the solubility of the polypeptide in a concentrated solution by forming an energetically unfavorable precipitating depleted layer around the polypeptide molecules [Weber (1991) Advances in Protein Chemistry, 41:1-36]. In addition to precipitants, other materials are sometimes added to the polypeptide crystallization solution. These include buffers to adjust the pH of the solution and salts to reduce the solubility of the polypeptide. Various precipitants are known in the art and include the following: ethanol, 3-ethyl-2-4 pentanediol, and many of the polyglycols, such as polyethylene glycol (PEG). The precipitating solutions can include, for example, 13-24% PEG 4000, 5-41% ammonium sulfate, and 1.0-1.5 M sodium chloride, and a pH ranging from 5-7.5. Other additives can include 0.1 M Hepes, 2-4% butanol, 0.1 M or 20 mM sodium acetate, 50-70 mM citric acid, 120-130 mM sodium phosphate, 1 mM ethylene diamine tetraacetic acid (EDTA), and 1 mM dithiothreitol (DTT). These agents are prepared in buffers and are added dropwise in various combinations to the crystallization buffer.

Commonly used polypeptide crystallization methods include the following techniques: batch, hanging drop, seed initiation, and dialysis. In each of these methods, it is important to promote continued crystallization after nucleation by maintaining a supersaturated solution. In the batch method, polypeptide is mixed with precipitants to achieve supersaturation, and the vessel is sealed and set aside until crystals appear. In the dialysis method, polypeptide is retained in a sealed dialysis membrane that is placed into a solution containing precipitant. Equilibration across the membrane increases the polypeptide and precipitant concentrations, thereby causing the polypeptide to reach supersaturation levels.

In the preferred hanging drop technique [McPherson (1976) J. Biol. Chem., 251:6300-6306], an initial polypeptide mixture is created by adding a precipitant to a concentrated polypeptide solution. The concentrations of the polypeptide and precipitants are such that, in this initial form, the polypeptide does not crystallize. A small drop of this mixture is placed on a glass slide that is inverted and suspended over a reservoir of a second solution. The system is then sealed. Typically, the second solution contains a higher concentration of precipitant or other dehydrating agent. The difference in the precipitant concentrations causes the protein solution to have a higher vapor pressure than the second solution. Since the system containing the two solutions is sealed, an equilibrium is established, and water from the polypeptide mixture transfers to the second solution. This equilibrium increases the polypeptide and precipitant concentration in the polypeptide solution. At the critical concentration of polypeptide and precipitant, a crystal of the polypeptide may form.

Another method of crystallization introduces a nucleation site into a concentrated polypeptide solution. Generally, a concentrated polypeptide solution is prepared and a seed crystal of the polypeptide is introduced into this solution. If the concentrations of the polypeptide and any precipitants are correct, the seed crystal will provide a nucleation site around which a larger crystal forms.

Yet another method of crystallization is an electrocrystallization method in which use is made of the dipole moments of protein macromolecules that self-align in the Hehnholtz layer adjacent to an electrode (see, e.g., U.S. Pat. No. 5,597,457, the disclosure of which is incorporated herein by reference in its entirety).

Some proteins may be recalcitrant to crystallization. However, several techniques are available to the skilled artisan to induce crystallization. For example, the removal of flexible polypeptide segments at the amino or carboxyl terminal end of the protein may facilitate production of crystalline protein samples. Removal of such segments can be done using molecular biology techniques or treatment of the protein with proteases such as trypsin, chymotrypsin, or subtilisin.

In diffraction experiments, a narrow and parallel beam of x-rays is taken from the x-ray source and directed onto the crystal to produce diffracted beams. The incident primary beams cause damage to both the macromolecule and solvent molecules. The crystal is, therefore, cooled (e.g., to −220° C. to −50° C.) to prolong its lifetime. The primary beam must strike the crystal from many directions to produce all possible diffraction spots, so the crystal is rotated in the beam during the experiment. The diffracted spots are recorded on a film or by an electronic detector. Exposed film has to be digitized and quantified in a scanning device, whereas the electronic detectors feed the signals they detect directly into a computer. Electronic area detectors significantly reduce the time required to collect and measure diffraction data. Each diffraction beam, which is recorded as a spot on film, is defined by three properties: the amplitude, which is measured from the intensity of the spot; the wavelength, which is set by the x-ray source; and the phase, which is lost in x-ray experiments. All three properties are needed for all of the diffracted beams in order to determine the positions of the atoms giving rise to the diffracted beams. One way of determining the phases is called Multiple Isomorphous Replacement (MIR), which requires the introduction of exogenous x-ray scatterers (e.g., heavy atoms such metal atoms) into the unit cell of the crystal. For a more detailed description of MIR, see U.S. Pat. No. 6,093,573 (column 15) the disclosure of which is incorporated herein by reference in its entirety.

Atomic coordinates refer to Cartesian coordinates (x, y, and z positions) derived from mathematical equations involving Fourier synthesis of data derived from patterns obtained via diffraction of a monochromatic beam of x-rays by the atoms (scattering centers) of biological macromolecule of interest in crystal form. Diffraction data are used to calculate electron density maps of repeating units in the crystal (unit cell). Electron density maps are used to establish the positions (atomic coordinates) of individual atoms within a crystal's unit cell. The absolute values of atomic coordinates convey spatial relationships between atoms because the absolute values ascribed to atomic coordinates can be changed by rotational and/or translational movement along x, y, and/or z axes, together or separately, while maintaining the same relative spatial relationships among atoms. Thus, a biological macromolecule (e.g., a protein) whose set of absolute atomic coordinate values can be rotationally or translationally adjusted to coincide with a set of prior determined values from an analysis of another sample is considered to have the same atomic coordinates as those obtained from the other sample.

Further details on x-ray crystallography can be obtained from co-pending U.S. application Ser. No. 10/486,278, U.S. Pat. No. 6,093,573 and International Application Nos. PCT/US99/18441, PCT/US99/11913, and PCT/US00/03745. The disclosures of all these patent documents are incorporated herein by reference in their entirety.

NMR Spectroscopy

While x-ray crystallography requires single crystals of a macromolecule of interest, NMR measurements are carried out in solution under near physiological conditions. However, NMR-derived structures are not as detailed as crystal-derived structures.

While the use of NMR spectroscopy was until relatively recently limited to the elucidation of the 3-D structure of relatively small molecules (e.g., proteins of 100-150 amino acid residues), recent advances including isotopic labeling of the molecule of interest and transverse relaxation-optimized spectroscopy (TROSY) have allowed the methodology to be extended to the analysis of much larger molecules, e.g., proteins with a molecular weight of 110 kDa [Wider (2000) BioTechniques, 29:1278-1294].

NMR uses radio-frequency radiation to examine the environment of magnetic atomic nuclei in a homogeneous magnetic field pulsed with a specific radio frequency. The pulses perturb the nuclear magnetization of those atoms with nuclei of nonzero spin. Transient time domain signals are detected as the system returns to equilibrium. Fourier transformation of the transient signal into a frequency domain yields a one-dimensional NMR spectrum. Peaks in these spectra represent chemical shifts of the various active nuclei. The chemical shift of an atom is determined by its local electronic environment. Two-dimensional NMR experiments can provide information about the proximity of various atoms in the structure and in three dimensional space. Protein structures can be determined by performing a number of two- (and sometimes 3- or 4-) dimensional NMR experiments and using the resulting information as constraints in a series of protein folding simulations.

More information on NMR spectroscopy including detailed descriptions of how raw data obtained from an NMR experiment can be used to determine the 3-D structure of a macromolecule can be found in: Protein NMR Spectroscopy, Principles and Practice, J. Cavanagh et al., Academic Press, San Diego, 1996; Gronenborn et al. (1990) Anal. Chem. 62(1):2-15; and Wider (2000), supra., the disclosures of all of which are incorporated herein by reference in their entirety

Any available method can be used to construct a 3-D model of a region of MUC1 and/or Abl of interest from the x-ray crystallographic and/or NMR data using a computer as described above. Such a model can be constructed from analytical data points inputted into the computer by an input device and by means of a processor using known software packages, e.g., HKL, MOSFILM, XDS, CCP4, SHARP, PHASES, HEAVY, XPLOR, TNT, NMRCOMPASS, NMRPIPE, DIANA, NMRDRAW, FELIX, VNMR, MADIGRAS, QUANTA, BUSTER, SOLVE, O, FRODO, or CHAIN. The model constructed from these data can be visualized via an output device of a computer, using available systems, e.g., Silicon Graphics, Evans and Sutherland, SUN, Hewlett Packard, Apple Macintosh, DEC, IBM, or Compaq.

Compounds

Compounds identified in any of the methods described herein, or any compound with appropriate activity useful in any of the methods described herein can include various chemical classes, though typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

In alternative embodiments, compounds can also include biomolecules including, but not limited to, peptides, polypeptides, peptidomimetics (e.g., peptoids), amino acids, amino acid analogs, saccharides, fatty acids, steroids, purines, pyrimidines, derivatives or structural analogues thereof, polynucleotides, and polynucleotide analogs.

Of particular interest as small molecule compounds are nucleic acid aptamers which are relatively short nucleic acid (DNA, RNA or a combination of both) sequences that bind with high avidity to a variety of proteins and inhibit the binding to such proteins of ligands, receptors, and other molecules. Aptamers are generally about 25-40 nucleotides in length and have molecular weights in the range of about 18-25 kDa. Aptamers with high specificity and affinity for targets can be obtained by an in vitro evolutionary process termed SELEX (systemic evolution of ligands by exponential enrichment) [see, for example, Zhang et al. (2004) Arch. Immunol. Ther. Exp. 52:307-315, the disclosure of which is incorporated herein by reference in its entirety]. For methods of enhancing the stability (by using nucleotide analogs, for example) and enhancing in vivo bioavailability (e.g., in vivo persistence in a subject's circulatory system) of nucleic acid aptamers see Zhang et al. (2004) and Brody et al. [(2000) Reviews in Molecular Biotechnology 74:5-13, the disclosure of which is incorporated herein by reference in its entirety].

Compounds can be identified from a number of potential sources, including: chemical libraries, natural product libraries, and combinatorial libraries comprised of random peptides, oligonucleotides, or organic molecules. Chemical libraries consist of random chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries.

Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Bioechnol. 8:701-707 (1997).

Identification of test compounds through the use of the various libraries herein permits subsequent modification of the test compound “hit” or “lead” to optimize the capacity of the “hit” or “lead” to inhibit the interaction between Abl and MUC1.

In addition, the inhibitory compounds can be antibodies, or antigen-binding antibody fragments, specific for MUC1 or Abl. Such antibodies will generally bind to, or close to: (a) the region of MUC1 to which Abl binds; (b) or the region on Abl to which MUC1 binds. However, as indicated above, the compounds can also act allosterically and so they can also bind to the proteins at positions other than, and even remote from, the binding sites for MUC1 (on Abl) and on Abl (for MUC1). As used throughout the present application, the term “antibody” refers to a whole antibody (e.g., IgM, IgG, IgA, IgD, or IgE) molecule that is generated by any one of a variety of methods that are known in the art. The antibody can be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice.

The antibody can be a purified or a recombinant antibody. Also useful for the methods described herein are antibody fragments and chimeric antibodies and humanized antibodies made from non-human (e.g., mouse, rat, gerbil, or hamster) antibodies. As used herein, the term “antibody fragment” refers to an antigen-binding fragment, e.g., Fab, F(ab′)₂, Fv, and single chain Fv (scFv) fragments. An scFv fragment is a single polypeptide chain that includes both the heavy and light chain variable regions of the antibody from which the scFv is derived. In addition, diabodies [Poljak (1994) Structure 2(12):1121-1123; Hudson et al. (1999) J. Immunol. Methods 23(1-2):177-189, the disclosures of both of which are incorporated herein by reference in their entirety] and intrabodies [Huston et al. (2001) Hum. Antibodies 10(3-4):127-142; Wheeler et al. (2003) Mol. Ther. 8(3):355-366; Stocks (2004) Drug Discov. Today 9(22): 960-966, the disclosures of all of which are incorporated herein by reference in their entirety] can be used in the methods described herein.

Antibody fragments that contain the binding domain of the molecule can be generated by known techniques. For example: F(ab′)₂ fragments can be produced by pepsin digestion of antibody molecules; and Fab fragments can be generated by reducing the disulfide bridges of F(ab′)₂ fragments or by treating antibody molecules with papain and a reducing agent. See, e.g., National Institutes of Health, 1 Current Protocols In Immunology, Coligan et al., ed. 2.8, 2.10 (Wiley Interscience, 1991) the disclosure of which is incorporated herein by reference in its entirety. scFv fragments can be produced, for example, as described in U.S. Pat. No. 4,642,334, the disclosure of which is incorporated herein by reference in its entirety.

Chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60. The disclosures of all these articles and patent documents are incorporated herein by reference in their entirety.

The compounds identified above can be synthesized by any chemical or biological method. The compounds identified above can also be pure, or may be in a heterologous composition (e.g., a pharmaceutical composition), and can be prepared in an assay-, physiologic-, or pharmaceutically-acceptable diluent or carrier (see Pharmaceutical Compositions and Methods of Treatment below). This composition can also contain additional compounds or constituents which do not bind to or inhibit the interaction between Abl and MUC1, but are useful in the application of various methods described herein (e.g., a composition may contain one or more additional genotoxic agents, see below).

Pharmaceutical Compositions and Methods of Treatment

The present disclosure also provides for pharmaceutical compositions comprising a therapeutically effective amount of a compound, or a pharmaceutically acceptable salt thereof together with a pharmaceutically acceptable carrier, diluent, or excipient therefor. A compound that has been screened by a method described herein and/or determined, for example, to (a) inhibit the interaction between MUC1 and Abl, (b) inhibit the phosphorylation of MUC1 by Abl, (c) stimulate translocation of Abl to the nucleus, (d) inhibit BCR-Abl, (e) induce apoptosis in a cell, or (f) inhibit the growth of a cancer cell (e.g., a colon cancer cell, a breast cancer cell, a prostate cancer cell, a lung cancer cell, a lymphoma), can be considered a candidate compound. A candidate compound that has been screened, e.g., in an in vivo model of cancer or inflammation and determined to have a desirable effect on the disorder, can be considered a candidate therapeutic agent. Candidate therapeutic agents, once screened in a clinical setting, are therapeutic agents. Candidate therapeutic agents and therapeutic agents can be optionally optimized and/or derivatized, and formulated with physiologically acceptable excipients to form pharmaceutical compositions.

Any of the compounds described herein can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. A compound of described herein can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Dosage units can also be accompanied by instructions for use.

The dose administered to a subject, in the context of the present disclosure, should be sufficient to affect a beneficial therapeutic response in the subject over time. The term “subject” refers to a member of the class Mammalia. Examples of mammals include, without limitation, humans or primates (e.g., chimpanzees, baboons, or monkeys), mice, rats, rabbits, guinea pigs, gerbils, hamsters, horses, livestock (e.g., cows, pigs, sheep, or goats), dogs, cats, or whales. In certain embodiments, the “subject” is a human (e.g., a human patient).

The dose will be determined by the efficacy of the particular compound employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present disclosure can be administered at a rate determined by factors that can include, but are not limited to, the pharmacokinetic profile of the compound, contraindicated drugs, and the side effects of the compound at various concentrations, as applied to the mass and overall health of the subject. Administration can be accomplished via single or divided doses.

Toxicity and therapeutic efficacy of such compounds can be determined by known pharmaceutical procedures in cell cultures or experimental animals (animal models of cancer, e.g., colon, breast, prostate, or lung cancer models, or animal models of neurologic disorders, e.g., Alzheimer's or Parkinson's Disease). These procedures can be used, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD50/ED₅₀. Compounds that exhibit high therapeutic indices are preferred. While compounds that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For a compound used as described herein (e.g., for treating cancer, neurologic disorder, or atherosclerotic lesion in a subject), the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Compounds that inhibit the growth of a cell, (i.e., a mammalian cell, a human cancer cell) can be any of the compounds described herein.

As defined herein, a therapeutically effective amount of a compound (i.e., an effective dosage) includes milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the inhibition of the cell growth (i.e., inhibition of the growth of a cancer cell). When one or more of these small molecules is to be administered to an animal (e.g., a human) to treat an infection or a cancer, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated. One in the art will also appreciate that certain additional factors may influence the dosage and timing required to effectively treat a subject, including but not limited to the severity of the disease or disorder, previous treatments, and other diseases present. Moreover, treatment of a subject with a therapeutically effective amount of a protein, polypeptide, or antibody can include a single treatment or can include a series of treatments.

The pharmaceutical compositions can be included in a container, pack, or dispenser together with instructions for administration.

Methods of Inhibiting an Interaction Between Abl and MUC1

Provided herein are in vitro, in vivo, and ex vivo methods of inhibiting an interaction between MUC1 and Abl. Since the binding of MUC1, or a phosphorylated form of MUC1, to Abl (a) sequesters Abl at the cell membrane, and (b) inhibits the ability of Abl to translocate to the nucleus and activate an apoptotic program following the genotoxic stress of a cell, such methods of inhibition can have general applicability in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, such methods can also be useful in the treatment of cancers.

Inhibition of the interaction between MUC1 and Abl can include inhibition of an interaction between MUC1 and any form of Abl (e.g., wild-type c-Abl, v-Abl, BCR-Abl, a drug resistant or multi-drug resistant variant of Abl, or any functional or MUC1-binding fragment of Abl (e.g., the SH2 domain of c-Abl as depicted in SEQ ID NO:5)) described herein. Similarly, MUC1, as referred to in the method, can include full-length, wild-type, mature MUC1 polypeptide (SEQ ID NO:1), the MUC1-cytoplasmic domain (MUC1-CD) (SEQ ID NO:2), or a functional or Abl-binding fragment of a MUC1 polypeptide (e.g., MUC1-CD-YTNP motif as depicted in SEQ ID NO:3 or a phosphorylated form thereof such as pYTNP). Cells useful in the methods described herein can include both prokaryotic (e.g., bacterial cells) and eukaryotic cells. Eukaryotic cells can include, for example, yeast, insect, plant, fish, reptile, and mammalian cells (e.g., mouse, rat, rabbit, guinea pig, dog, cat, pig, horse, goat, cow, whale, monkey, or human). The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines.

The methods of inhibiting an interaction between MUC1 and Abl can optionally include a step of identifying a cell as expressing MUC1. Such identification can include, for example, identifying (or detecting) whether a cell expresses MUC1 mRNA or MUC1 protein. Suitable methods of identifying (or detecting) the expression of MUC1 protein or MUC1 mRNA are well known to those of skill in the art, and are described herein. These methods can include, for example, SDS-polyacrylamide gel electrophoresis/western blotting techniques using antibodies specific for MUC1 (for detection of protein), or RT-PCR or northern blotting techniques for detection of mRNA expression. The cell can be any cell that expresses MUC1, including any cell described above and any cells that express an endogenous MUC1 or a cell that expresses a recombinant or exogenous MUC1 mRNA or polypeptide.

Compounds useful in the methods of inhibiting an interaction between MUC1 and Abl can include any of the compounds described herein, or any other compounds with the appropriate inhibitory activity. Suitable compounds can include small molecules, antibodies, antibody fragments, polypeptides, or peptidomimetics. Compounds can also include nucleic acids, for example, nucleic acids that inhibit the mRNA or protein expression of MUC1 or Abl (e.g., siRNA or anti-sense nucleic acids; see “Methods of Inhibiting Abl”). Other exemplary compounds for use in the methods include MUC1 or Abl polypeptides or their functional fragments. Examples of functional fragments of MUC1 include, for example, the MUC1-CD (SEQ ID NO:2) or MUC1-CD-YTNP (SEQ ID NO:3), or fragments of the MUC1-CD that contain pYTNP (e.g., a polypeptide fragment of MUC1-CD (SEQ ID NO:2) containing amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70). Examples of functional fragments of Abl polypeptide include all or part of the SH2 domain of c-Abl (SEQ ID NO:5) or all or part of the SH3 of c-Abl (SEQ ID NO:8).

While the invention is not limited by any particular mechanism of action, it appears that the binding of MUC1 to Abl sequesters Abl at the cell membrane and prevents the ability of Abl to translocate to the nucleus and activate programmed cell death in response to genotoxic insult to a cell. Thus, co-culturing a cell in the presence of, or further administering to a subject (e.g., a human patient), an inhibitor of an interaction between MUC1 and Abl and a genotoxic agent can increase the efficacy of the genotoxic agent. In some embodiments of the methods of inhibiting the interaction between MUC1 and Abl, the cells or subjects may be further treated with one or more additional genotoxic agents. Such genotoxic agents can include, but are not limited to, one or more chemotherapeutic agents, or one or more forms of ionizing radiation. Genotoxic chemotherapeutic agents can include carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatinum, adriamycin, or an analog of any of the aforementioned. Forms of ionizing radiation can include, for example, beta radiation, gamma radiation, X-radiation, UV-radiation, or infra-red radiation.

Cells or subjects may be further contacted with one or more kinase inhibitors (e.g., antibodies that inhibit receptor tyrosine kinases (e.g., Trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Inhibiting an Interaction Between MUC1 and Abl

Provided herein is an in vitro method of inhibiting an interaction between MUC1 and Abl. The method can be useful, for example, in scientific studies investigating the role of MUC1 in the c-Abl signal transduction cascade, or any other scientific studies in which inhibiting the interaction between MUC1 and Abl can be beneficial. The method can also be useful as a further cell-based screening step, in e.g., a drug screening cascade, following the biochemical (e.g., a cell-free method of identifying a compound that inhibits the binding of Abl to MUC1 described above) identification of a compound that inhibits the binding of Abl to MUC1.

The method can include the steps of: optionally identifying a cancer cell as expressing MUC1 (e.g., a cell expressing MUC1 protein or MUC1 mRNA), and culturing the cell with a compound that inhibits the interaction between MUC1 and Abl. The cell can be any of the cells described herein (e.g., see above). Methods for identifying or detecting a cell as expressing MUC1 mRNA or protein are well known to those in the art and are described above. Suitable concentrations of the inhibitory compound can be elucidated through routine experimentation and such optimization is well known to one of skill in the art. As described above, the cell may be co-cultured with one or more additional genotoxic agents.

Methods of determining or detecting the inhibition of an interaction between MUC1 and Abl are well known to those of skill in the art, and include, for example, in vitro and in situ methods. One method of determining inhibition of the interaction between MUC1 and Abl is an immunoprecipitation method and is set forth in the Examples below. Briefly, cells cultured in the presence of an inhibitory compound can be washed and harvested from the culture vessel. The cells can then be lysed using non-denaturing buffers that preserve protein-protein interactions, for example, buffers containing Nonidet-40 (NP-40) or Triton X-100 detergents. The lysates can then be clarified using, for example, centrifugation to remove insoluble debris. Clarified lysates can then be subjected to immunoprecipitation by adding to the lysate an antibody specific for either Abl or MUC1 for a time sufficient to allow for the binding of the antibody to its cognate antigen. Antibody-protein complexes are isolated from the lysate solution by coupling the complexes to solid support matrices. Examples of such solid support matrices include insoluble beads conjugated to anti-IgG antibodies or other antibody-binding moieties, for example, bacterial Protein-A or Protein-G. Isolated immunocomplexes can then be solubilized in Laemmli buffer optionally containing reducing agent and subjected to SDS-polyacrylamide gel electrophoresis (SDS-PAGE). Immunoblotting of the samples using antibodies specific for one or both of MUC1 and Abl can then determine whether a compound has inhibited the interaction between MUC1 and Abl. A reduced amount of Abl protein associated with an anti-MUC1 antibody immunoprecipitation in cells treated with a compound as compared to the amount of Abl associated with the MUC1 immunoprecipitates from lysates of cells not treated with the compound indicates that the compound has inhibited the interaction of the two proteins. Similarly, a reduced amount of MUC1 protein associated with an anti-Abl antibody immunoprecipitation from cells treated with a compound as compared to the amount of MUC1 protein associated with the Abl immunoprecipitates from lysates of cells not treated with the compound indicates that the compound has inhibited the interaction of the two proteins.

Another method of determining inhibition of an interaction between MUC1 and Abl is an in situ staining method. Immunostaining methods are well known to those of skill in the art and include embodiments where the cells are still viable (e.g., confocal microscopy of live cells) or staining of fixed cells (e.g., immunohistochemistry). Examples of such methods are set forth in the Examples below. Antibodies specific for MUC1 and Abl polypeptides are applied (e.g., administered, delivered, contacted) to cells. The antibodies are independently labeled with a different detectable label (e.g., a different colored fluorophore (e.g., rhodamine, Texas red, FITC, Green fluorescent protein, Cy3, Cy5) such that they can be readily and easily distinguished from one another. Use of an appropriate microscope (e.g., a confocal microscope) with the appropriate optical filters can identify the position of the labeled antibodies in a given cell. When each of the positions of the two proteins are determined (i.e., the location of their respective detectable label within the cell as determined by antibody binding), if they are found to occupy the same space, the two proteins are said to co-localize. Thus, when two proteins co-localize in the absence of a compound but do not co-localize in the presence of a compound, this can indicate that the compound has inhibited the interaction between the two proteins. Optionally the cells can be fixed, for example, using paraformaldehyde or formaldehyde, and permeabilized using a detergent (e.g., Triton-X100).

Since it appears that the binding of MUC1 to Abl prevents the localization of Abl to the nucleus of a cell, inhibiting the interaction between MUC and Abl can also be determined by detecting the physiologic localization of Abl (i.e., inhibition of the interaction of MUC1 and Abl would result in increases nuclear Abl). Methods of detecting the subcellular localization of Abl are well known to those of ordinary skill in the art (see, for example, the Example section below). Thus, more nuclear localization of Abl in the presence of an inhibitory compound, for example, as compared to the nuclear localization in the absence of the inhibitory compound indicates that the compound has promoted the nuclear localization of Abl. Nuclear localization of Abl can be detected, for example, by cell fractionation (i.e., detecting the amount of Abl in a cytosolic versus a nuclear extract prepared from the same source of cells, see the Examples section below) and immunoblotting or ELISA (see, Examples below). Alternatively, localization of Abl can be done in situ, generally by methods including, but not limited to: (i) fixing the cells; (ii) treatment of the fixed cells with detectably-labeled antibodies specific to Abl; and (iii) detecting the signal produced by the detectable label using any of a number of methods known to those in the art, including fluorescence-activated cell sorting (FACS) and confocal microscopy (see, Lin et al. (1995) J. Biol. Chem. 270(24):14255-14258; Tsukahara et al. (1999) J. Virol. 73(10):7981-7987). The detectable label can be conjugated to the first antibody (the primary antibody which specifically recognizes Abl) or to a secondary antibody which is capable of binding to the first antibody. Alternatively, the first antibody can be conjugated to the first member of a binding pair (i.e., strepavidin or biotin) and the second member of the binding pair can be linked to the detectable moiety. The detectable moiety can include radiolabels (e.g., ¹²⁵I, ³⁵S, ³³P, or ³²P), fluorescent labels (e.g., texas red, fluorescein), a luminescent moiety (e.g., a lanthanide), or a one or more members of a FRET pair.

Since it appears that Abl mediates programmed cell death in response to genotoxic stress of a cell, another method of determining the inhibition of an interaction between MUC1 and Abl is detecting an increased inhibition of cell growth or apoptosis of a cell in the presence of genotoxic agents (e.g., adriamycin or cisplatin) (see, for example, the Examples section below). Cells are generally plated on solid support matrix (e.g., a plastic tissue culture plate or a multiwell (96 or 386-well) tissue culture plate) and grown in appropriate medium. Cells are then co-cultured in the absence or presence of an appropriate inhibitory compound and a known genotoxic agent. Often, a control compound (e.g., a known inhibitor of known concentration) is also added to a set of cells as an internal standard. Often, a set of cells is grown in the presence of a carrier, buffer, or solvent, in which the compound is delivered. Methods of detecting (e.g., determining or measuring) cell growth inhibition by a compound are myriad and well known to those of ordinary skill in the art. These methods can include, for example, counting the number of cells remaining in the well after the period of treatment with the compound. In this method, cells can be trypsinized from the plate, washed, stained with a dye (e.g., typan blue), and counted using a microscope or mechanical cell counter (Beckman-Coulter Z1™ Series COULTER COUNTER® Cell and Particle Counter). Since dyes like trypan blue are only taken up by dead or dying cells, this method allows for discrimination (i.e., blue or white cell) between viable and non-viable cells in a population. Another method for determining cell growth inhibition in the presence of an inhibitory compound (e.g., any one of the compositions described herein) following treatment is a metabolic assay, for example, an MTT-metabolic assay (Invitrogen, USA). MTT diphenyltetrazolium bromide, is a tetrazolium salt (yellowish) that is cleaved to formazan crystals by the succinate dehydrogenase system which belongs to the mitochondrial respiratory chain, and is only active in viable cells. The mitochondrial succinate dehydrogenase reduces the MTT crystals into purple formazan in the presence of an electron coupling reagent. Following the treatment of the cells with a compound, the cells are exposed to the MTT reagent and the more viable cells are present in a well, the more formazan dye is produced. Extent of formazan dye can be measured, for example, using a spectrophotometer. Other commonly used methods of detecting cell growth inhibition include the monitoring of DNA synthesis. Cells grown, for example, in the presence or absence of compound are also treated with a nucleotide anolog that can incorporate into the DNA of the cell upon cell division. Examples of such nucleotide analogs include, for example, BrdU or ³H-Thymidine. In each case, the amount of label incorporated into the cells (grown in the presence and absence of a given inhibitory agent) is quantitated, and the amount of label incorporation is directly proportional to the amount of cell growth in the population of cells.

As described below in the Examples, cell growth inhibition, as a reflection of the inhibition of an interaction between MUC1 and Abl, can be determined by detecting cell death. Such methods are well known to those of skill in the art, and include propidium iodide staining of genomic DNA, or commercially available kits, such as, In Situ Cell Death Detection ELISA Kit (Roche, Indianapolis, Ind.); and APO-Direct, APO-BRDU, or Annexin-FITC Apoptosis Kit (BD-Pharmingen, San Diego, Calif.). Such methods and kits for determining programmed cell death can optionally be used in conjunction with fluorescence-activated cell sorting (FACS) analysis. Examples of the methods and machines (instruments) useful for such methods are described in the Examples section below.

In a preferred embodiment, any of the in vitro methods for detecting inhibition of the interaction between MUC1 and Abl (in vivo or in vitro) can be performed in any format that allows for rapid preparation, processing, and analysis of multiple reactions. This can be, for example, in multi-well assay plates (e.g., 96 wells or 386 wells). Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

In Vivo Methods of Inhibiting an Interaction Between MUC1 and Abl

The disclosure features a method of treating a subject having, or is at risk of developing, a cancer, which includes the steps of: optionally identifying a subject as having, or at risk of developing, a cancer comprising one or more cancer cells expressing MUC1; and delivering to the subject a composition comprising a compound that inhibits the interaction between MUC1 and Abl.

The disclosure also features a method for treating a subject having, suspected of having, or at risk of developing, an inflammatory condition. The method includes the steps of: optionally identifying a subject as having, suspected of having, or at risk of developing, an inflammatory condition mediated by one or more immune cells expressing MUC1; and delivering to the subject a composition comprising a compound that inhibits the interaction between MUC1 and Abl.

In one in vivo approach, a compound that inhibits binding of MUC1 to Abl is administered to a subject. The subject can be any mammal, e.g., a human (e.g., a human subject) or a primate (e.g., chimpanzee, baboon, or monkey), mouse, rat, rabbit, guinea pig, gerbil, hamster, horse, a type of livestock (e.g., cow, pig, sheep, or goat), a dog, cat, or a whale. Generally, the compounds of the invention will be suspended in a pharmaceutically-acceptable carrier (e.g., physiological saline) and administered orally or injected intravenously, subcutaneously, intramuscularly, intrathecally, intraperitoneally, intrarectally, intravaginally, intranasally, intragastrically, intratracheally, or intrapulmonarily. They can also be delivered directly to tumor cells, e.g., to a tumor or a tumor bed following surgical excision of the tumor, in order to kill any remaining tumor cells. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001 mg/kg-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Alternatively, where an inhibitory compound is a polypeptide, a polynucleotide containing a nucleic acid sequence encoding the polypeptide can be delivered to appropriate cells in a mammal. Expression of the coding sequence can be directed to any cell in the body of the subject. However, expression can be directed to cells in the vicinity of the tumor cells or immune cells whose proliferation it is desired to inhibit. Expression of the coding sequence can be directed to the tumor cells themselves. This can be achieved by, for example, the use of polymeric, biodegradable microparticle or microcapsule delivery devices known in the art. Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73:479, the disclosure of which is incorporated herein by reference in its entirety]. Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the polypeptide of interest with an initiator methionine and optionally a targeting sequence is operatively linked to a promoter or enhancer-promoter combination. Short amino acid sequences can act as signals to direct proteins to specific intracellular compartments. Such signal sequences are described in detail in U.S. Pat. No. 5,827,516, the disclosure of which is incorporated herein by reference in its entirety.

Enhancers provide expression specificity in terms of time, location, and level. Unlike a promoter, an enhancer can function when located at variable distances from the transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. Promoters of interest include but are not limited to the cytomegalovirus hCMV immediate early gene, the early or late promoters of SV40 adenovirus, the lac system, the LM system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors, the adenoviral E1b minimal promoter, or the thymidine kinase minimal promoter. The DF3 enhancer can be particularly useful for expression of an inhibitory compound in cells that naturally express MUC1, for example, normal epithelial cells or malignant epithelial cells (carcinoma cells), e.g., breast cancer cells [see U.S. Pat. Nos. 5,565,334 and 5,874,415, the disclosures of which are incorporated herein by reference in their entirety]. The coding sequence of the expression vector is operatively linked to a transcription terminating region.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased proliferation of cancer cells) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to approximately 10¹² copies of the polynucleotide molecule. This dose can be repeatedly administered, as needed. Routes of administration can be any of those listed above.

Ex Vivo Methods of Inhibiting an Interaction Between MUC1 and Abl

An ex vivo strategy can involve transfecting or transducing cells obtained from the subject with a polynucleotide encoding a polypeptide that inhibit an interaction between MUC1 and Abl. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the inhibitory polypeptide for as long as they survive in the subject. Alternatively, tumor cells or immune cells, preferably obtained from the subject (autologous) but potentially from a subject of the same species other than the subject (allogeneic), can be transfected or transformed by a vector encoding the inhibitory polypeptide. The tumor cells, preferably treated with an agent (e.g., ionizing irradiation) that ablates their proliferative capacity, are then introduced into the patient, where they secrete the polypeptide.

The ex vivo methods include the steps of harvesting cells from a subject, culturing the cells, transducing them with an expression vector, and maintaining the cells under conditions suitable for expression of the polypeptide that inhibits binding of MUC1 to Abl. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can be selected, for example, for expression of the coding sequence or of a drug resistance gene. The cells may then be lethally irradiated (if desired) and injected or implanted into the patient.

Methods of Inhibiting MUC1 Phosphorylation by Abl

Provided herein are in vitro, in vivo, and ex vivo methods of inhibiting the phosphorylation of MUC1 by Abl. While the invention is not limited to any particular mechanism of action, it appears the binding of MUC1, or a phosphorylated form of MUC1, to Abl (a) sequesters Abl at the cell membrane, and (b) inhibits the ability of Abl to translocate to the nucleus and activate an apoptotic program following the genotoxic stress of a cell. Since the binding between MUC1 and Abl is positively regulated by phosphorylation of MUC1 by Abl, methods of inhibiting MUC1 phosphorylation by Abl can have general applicability in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, such methods can also be useful in the treatment of cancers.

Inhibition of the phosphorylation of MUC1 by Abl can include inhibition of wild-type c-Abl, v-Abl, BCR-Abl, a drug resistant or multi-drug resistant variant of Abl, or any functional fragments of Abl (e.g., fragments of Abl containing a functional kinase domain) described herein. Similarly, MUC1, as referred to in the method, can include full-length, wild-type, mature MUC1 polypeptide (SEQ ID NO:1), the MUC1-cytoplasmic domain (MUC1-CD) (SEQ ID NO:2), or a functional or Abl-binding fragment of a MUC1 polypeptide (e.g., the phosphorylated form of the MUC1-CD-YTNP motif (SEQ ID NO:3)). Cells useful in the methods described herein can include both prokaryotic (e.g., bacterial cells) and eukaryotic cells. Eukaryotic cells can include, for example, yeast, insect, plant, fish, reptile, and mammalian cells (e.g., mouse, rat, rabbit, guinea pig, dog, cat, pig, horse, goat, cow, whale, monkey, or human). The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines.

The methods of inhibiting the phosphorylation of MUC1 by Abl can optionally include a step of identifying a cell as expressing MUC1. Such identification can include, for example, identifying (or detecting) whether a cell expresses MUC1 mRNA or MUC1 protein. Suitable methods of identifying (or detecting) the expression of MUC1 protein or MUC1 mRNA are well known to those of skill in the art, and are described herein. These methods can include, for example, SDS-polyacrylamide gel electrophoresis/western blotting techniques using antibodies specific for MUC1 (for detection of protein), or RT-PCR or northern blotting techniques for detection of mRNA expression. The cell can be any cell that expresses MUC1, including any cell described above and any cells that express an endogenous MUC1 or a cell that expresses a recombinant or exogenous MUC1 mRNA or polypeptide.

Compounds useful in the methods of inhibiting the phosphorylation of MUC1 by Abl can include any of the compounds described herein, or any other compounds with the appropriate inhibitory activity. Suitable compounds can include small molecules, antibodies, an antibody fragments, polypeptides, or a peptidomimetics. Compounds can also include nucleic acids, for example, nucleic acids that inhibit the mRNA or protein expression of MUC1 or Abl (e.g., siRNA or anti-sense nucleic acids; see “Methods of Inhibiting Abl”). Other exemplary compounds for use in the methods include MUC1 or Abl polypeptides or their functional fragments. Examples of functional fragments of MUC1 include, for example, the MUC1-CD (SEQ ID NO:2) or MUC1-CD-YTNP (SEQ ID NO:3), or fragments of the MUC1-CD that contain pYTNP (e.g., a polypeptide containing amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70). Examples of functional fragments of Abl polypeptide include all or part of the SH2 domain of c-Abl (SEQ ID NO:5) or all or part of the SH3 domain of c-Abl (SEQ ID NO:8).

Furthermore, Abl kinase inhibitors are well known to those in the art, and include, for example, Gleevec (imatinib mesylate, also known as STI571; Peggs, et al. (2004) Clin Exp Med. 4(1):1-9; Drucker et al. (2004) Adv Cancer Res. 91:1-30), AMN107 (Weisberg et al. (2006) Br. J. Cancer 94(12):1765-9); and dasatinib (2006) N Engl. J. Med. 354(24):2531-41).

The binding of MUC1 to Abl sequesters Abl at the cell membrane and prevents the ability of Abl to translocate to the nucleus and activate programmed cell death in response to genotoxic insult to a cell. Thus, co-culturing a cell in the presence of, or further administering to a subject (e.g., a human patient), an inhibitor of an interaction between MUC1 and Abl and a genotoxic agent can increase the efficacy of the genotoxic agent. In some embodiments of the methods of inhibiting the interaction between MUC1 and Abl, the cells or subjects may be further treated with one or more additional genotoxic agents. Such genotoxic agents can include any of the genotoxic agents described herein. Cells or subjects may be further contacted with one or more kinase inhibitors (e.g., antibodies that inhibit receptor tyrosine kinases (e.g., Trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Inhibiting Phosphorylation of MUC1 by Abl

Provided herein are in vitro methods of: inhibiting the phosphorylation of MUC1 by Abl. The methods can be useful, for example, in scientific studies investigating the role of MUC1 in the c-Abl signal transduction cascade, or any other scientific studies in which inhibiting the phosphorylation of MUC1 by Abl can be beneficial. The method can also be useful as a further cell-based screening step, in e.g., a drug screening cascade, following the biochemical (e.g., a cell-free method of identifying a compound that inhibits the binding of Abl to MUC1 described above) identification of a compound that inhibits the binding of Abl to MUC1.

The method can include the steps of optionally identifying a cancer cell as expressing MUC1 (e.g., a cell expressing MUC1 protein or MUC1 mRNA), and culturing the cell with a compound that inhibits the phosphorylation of MUC1 by Abl. The cell can be any of the cells described herein (e.g., see above). Methods for identifying or detecting a cell as expressing MUC1 mRNA or protein are well known to those in the art and are described above. Suitable inhibitory concentrations of the compound can be elucidated through routine experimentation and such optimization is well known to one of skill in the art. As described above, the cell may be co-cultured with one or more additional genotoxic agents.

Methods for determining inhibition of the phosphorylation of MUC1 by Abl are well known to those of skill in the art (and set forth in the Examples below) and include, for example, western blotting, immunostaining, or immunohistochemistry using antibodies specific for phosphorylated MUC1. Antibodies useful for these methods include antibodies that specifically recognize a phosphorylated form of MUC1 or the MUC1-CD. Alternatively, antibodies that generally recognize a phosphotyrosine motif can also be used in conjunction with another antibody specific for MUC1. For example, MUC1 protein can be immunoprecipitated from lysate prepared from a cell treated by the method, subjected to SDS-PAGE and immunoblotted using an anti-phospho-tyrosine antibody. The relative position of the phosphorylated band can be compared to the position of MUC1 as determined by immunoblot using the MUC1-specific antibodies. Using these methods, a reduction in the amount of signal produced from a phosphorylated MUC1 detection moiety (e.g., a detectably-labeled anti-phospho-MUC1 antibody) in the presence of a compound, as compared to the amount of signal produced in the absence of a compound indicates that a compound has inhibited the phosphorylation of MUC1. Alternatively, antibodies that recognize non-phosphorylated MUC1 may be amenable for detecting the inhibition of MUC1 phosphorylation by Abl as they can be used to detect changes in protein mobility consistent phosphorylation. For example, phosphorylated MUC1 protein can run faster or slower in an SDS-PAGE gel as compared to the mobility of unphosphorylated MUC1.

Since inhibition of phosphorylation of MUC1 by Abl can also result in a decrease in the association between MUC1 and Abl, any method for detecting, for example, the interaction between MUC1 and Abl or the localization of Abl (e.g., by methods of immunoprecipitation, immunofluorescence, immunohistochemistry) described herein can also be used to verify that inhibition of MUC1 phosphorylation by Abl has occurred.

In Vivo Methods of Inhibiting the Phosphorylation of MUC1 by Abl

The disclosure features in vivo methods of inhibiting the phosphorylation of MUC by Abl, which include the steps of optionally identifying a subject as having, or at risk of developing, a cancer comprising one or more cancer cells expressing MUC1; and delivering to the subject a composition comprising a compound that inhibits the phosphorylation of MUC1 by Abl.

The disclosure also features in vivo methods of inhibiting the phosphorylation of MUC1 by Abl, which include the steps of optionally identifying a subject as having, or at risk of developing, an inflammatory condition mediated by one or more immune cells expressing MUC1 and/or Abl; and delivering to the subject a composition comprising a compound that inhibits the phosphorylation of MUC1 by Abl.

Any compound useful in the inhibition of the phosphorylation of MUC1 by Abl described herein can be delivered (e.g., administered) to a subject in any method described above.

Ex Vivo Methods of Inhibiting the Phosphorylation of MUC1 by Abl

An ex vivo strategy can involve transfecting or transducing cells obtained from the subject (or from another subject) with a polynucleotide encoding a polypeptide that inhibits the phosphorylation of MUC1 by Abl. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide that inhibits the phosphorylation of MUC1 by Abl. These methods are known in the art of molecular biology and suitable methods are described above.

Methods of Inducing Apoptosis in a Cell

Provided herein are in vitro, in vivo, and ex vivo methods of inducing apoptosis in a cancer cell by inhibiting expression of MUC1. While the invention is not limited by any particular mechanism of action, since the binding of MUC1, or a phosphorylated form of MUC1, to Abl (a) sequesters Abl at the cell membrane, and (b) inhibits the ability of Abl to translocate to the nucleus and activate an apoptotic program following the genotoxic stress of a cell, such methods of inhibiting expression of MUC1 can have general applicability in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, the methods can be useful in treating certain cancers in a subject. Inhibition of MUC1 expression can include inhibition of MUC1 mRNA or MUC1 protein expression. Cells useful in the methods described herein can include, for example, insect, plant, fish, reptile, and mammalian cells (e.g., mouse, rat, rabbit, guinea pig, dog, cat, pig, horse, goat, cow, whale, monkey, or human). The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines. Subjects can include, for example, insects, plants, fish, reptiles, and mammals (e.g., mice, rats, rabbits, guinea pigs, dogs, cats, pigs, horses, goats, cows, whales, monkeys, or humans (e.g., human patients)).

Where the methods of inducing apoptosis include a step of identifying a cell as expressing MUC1 and Abl, such identification can include, for example, identifying (or detecting) whether a cell expresses MUC1 and Abl mRNA or MUC1 protein. Suitable methods of identifying (or detecting) the expression of MUC1 and Abl protein or MUC1 mRNA are well known to those of skill in the art, and are described herein. These methods can include, for example, SDS-polyacrylamide gel electrophoresis/western blotting techniques using antibodies specific for MUC1 and Abl (for detection of protein), or RT-PCR or northern blotting techniques for detection of mRNA expression. The cell can be any cell that expresses MUC1 and Abl, including any cell described above, any cell that expresses an endogenous MUC1 or a cell that expresses a recombinant or exogenous MUC1 and Abl mRNA or polypeptides.

Compounds useful in the methods (e.g., inhibiting the expression of MUC1) can include any of the compounds described herein, or any other compounds with the appropriate inhibitory activity. Suitable compounds can include small molecules, antibodies, antibody fragments, polypeptides, or a peptidomimetics. Compounds can also include nucleic acids, for example, nucleic acids that inhibit the mRNA or protein expression of MUC1 or Abl (e.g., siRNA or anti-sense nucleic acids; see “Methods of Inhibiting Abl”).

In some embodiments of the method, co-culturing a cell in the presence of, or co-delivering (co-administering) to a subject, an inhibitor of MUC1 expression and one or more genotoxic agents can increase the efficacy of the one or more genotoxic agents. Such genotoxic agents can include, but are not limited to, one or more chemotherapeutic agents, or one or more forms of ionizing radiation. Genotoxic chemotherapeutic agents can include carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, cisplatinum, adriamycin, or an analog of any of the aforementioned. Forms of ionizing radiation can include, for example, beta radiation, gamma radiation, X-radiation, UV-radiation, or infra-red radiation. Cells may be further contacted with and subjects may be further administered one or more kinase inhibitors (e.g., antibodies that inhibit receptor tyrosine kinases (e.g., Trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Inducing Apoptosis in a Cell

Provided herein are in vitro methods of inducing apoptosis in a cell. The methods can be useful, for example, in scientific studies investigating the role of MUC1 in apoptosis mechanisms, or any other scientific studies in which inducing apoptosis in cell an be beneficial. The method can also be useful as a further cell-based screening step, in e.g., a drug screening cascade, following a biochemical (e.g., a cell-free method of identifying a compound that inhibits the binding of Abl to MUC1 described above) identification of a compound that inhibits, e.g., the binding of Abl to MUC1.

The in vitro method of inducing apoptosis in a cell includes the steps of: optionally identifying a cell as expressing MUC1 and Abl, and culturing the cell with a compound that inhibits the expression of MUC1.

Suitable methods of determining inhibition of the expression of MUC1 protein or MUC1 mRNA are well known to those of skill in the art, and are described herein. These methods can include, for example, SDS-polyacrylamide gel electrophoresis/western blotting techniques using antibodies specific for MUC1 (for detection of protein), or RT-PCR or northern blotting techniques for detection of mRNA expression.

Methods for determining apoptosis in a cell are also well known to those in the art and are described above.

In Vivo Methods of Inducing Apoptosis in a Cell

The disclosure features an in vivo method of inducing apoptosis in a cell, which includes the steps of: optionally identifying a subject as having, or at risk of developing, a cancer comprising one or more cancer cells expressing MUC1 and Abl; and delivering to the subject a composition comprising a compound that inhibits the expression of MUC1.

Any compound useful in the inhibition of MUC1 expression described herein can be delivered (e.g., administered) to a subject in any method described above.

Ex Vivo Methods of Inducing Apoptosis in a Cell

An ex vivo strategy can involve transfecting or transducing cells obtained from the subject (or from another subject) with a polynucleotide encoding a polypeptide that inhibits the expression of MUC1. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide that inhibits the expression of MUC1. These methods are known in the art of molecular biology and suitable methods are described above.

Methods of Stimulating Abl Translocation to the Cell Nucleus

Provided herein are in vitro, in vivo, and ex vivo methods of stimulating the translocation of Abl to the nucleus of a cell. While the invention is not limited to any particular mechanism of action, since the binding of MUC1, or a phosphorylated form of MUC1, to Abl (a) sequesters Abl at the cell membrane, and (b) inhibits the ability of Abl to translocate to the nucleus (e.g., the translocation of Abl from the cytoplasm to the nucleus) and activate an apoptotic program following the genotoxic stress of a cell, such methods of inhibition can have general applicability in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, such methods can also be useful, for example, in the treatment of cancers.

Stimulating the translocation of Abl to the nucleus of a cell can include wild-type c-Abl, v-Abl, BCR-Abl, a drug resistant or multi-drug resistant variant of Abl, or any functional or MUC1-binding fragment of Abl (e.g., the SH2 domain of c-Abl as depicted in SEQ ID NO:5, or the SH3 domain of c-Abl as depicted in SEQ ID NO:8) described herein. Similarly, MUC1, as referred to in the method, can include full-length, wild-type, mature MUC1 polypeptide (SEQ ID NO:1), the MUC1-cytoplasmic domain (MUC1-CD) (SEQ ID NO:2), or a functional or Abl-binding fragment of a MUC1 polypeptide (e.g., a phosphorylated form of MUC1-CD-YTNP (SEQ ID NO:3) such as pYTNP). Cells useful in the methods described herein include, for example, yeast, insect, plant, fish, reptile, and mammalian cells (e.g., mouse, rat, rabbit, guinea pig, dog, cat, pig, horse, goat, cow, whale, monkey, or human). The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines.

The methods of stimulating the translocation of Abl to the nucleus of a cell can optionally include a step of identifying a cell as expressing MUC1. Such identification can include, for example, identifying (or detecting) whether a cell expresses MUC1 mRNA or MUC1 protein. Suitable methods of identifying (or detecting) the expression of MUC1 protein or MUC1 mRNA are well known to those of skill in the art, and are described herein. The cell can be any cell that expresses MUC1, including any cell as described above.

Compounds useful in the methods of inhibiting an interaction between Abl and MUC1 can include any of the compounds described herein, or any other compounds with the appropriate inhibitory activity. Suitable compounds can include small molecules, antibodies, an antibody fragments, polypeptides, or a peptidomimetics. Compounds can also include nucleic acids, for example, nucleic acids that inhibit the mRNA or protein expression of MUC1 or Abl (e.g., siRNA or anti-sense nucleic acids; see “Methods of Inhibiting Abl”). Other exemplary compounds for use in the methods include MUC1 or Abl polypeptides or their functional fragments. Examples of functional fragments of MUC1 include, for example, the MUC1-CD (SEQ ID NO:2) or a phosphorylated form of the MUC1-CD-YTNP (SEQ ID NO:3) motif such as pYTNP. Examples of functional fragments of Abl polypeptide include all or part of the SH2 domain of c-Abl (SEQ ID NO:5) or all or part of the SH3 domain of c-Abl (SEQ ID NO:8).

It appears that the binding of MUC1 to Abl sequesters Abl at the cell membrane and prevents the ability of Abl to translocate to the nucleus and activate programmed cell death in response to genotoxic insult to a cell. Thus, co-culturing a cell in the presence of, or further administering to a subject (e.g., a human patient), a compound that stimulates the translocation of Abl to the nucleus and a genotoxic agent can increase the efficacy of the genotoxic agent. In some embodiments of the methods stimulating the translocation of Abl to the nucleus of a cell, the cells or subjects may be further treated with one or more additional genotoxic agents or chemotherapeutic agents. Such genotoxic agents or chemotherapeutic agents can include any of the genotoxic or chemotherapeutic agents described herein. Cells or subjects may be further contacted with one or more kinase inhibitors (e.g., antibodies that inhibit receptor tyrosine kinases (e.g., Trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Stimulating the Translocation of Abl to the Nucleus of a Cell

Provided herein are in vitro methods of stimulating the translocation of Abl to the nucleus of a cell. The method can be useful, for example, in scientific studies investigating the role of MUC1 in the c-Abl signal transduction cascade, or any other scientific studies in which inhibiting the translocation of Abl to the nucleus of a cell can be beneficial. The methods can also be useful as further cell-based screening steps, in e.g., a drug screening cascade, following the biochemical (e.g., a cell-free method of identifying a compound that inhibits the binding of Abl to MUC1 described above) identification of a compound that inhibits the binding of Abl to MUC1.

The methods can include the steps of optionally identifying a cancer cell as expressing MUC1 (e.g., a cell expressing MUC1 protein or MUC1 mRNA), and culturing the cell with a compound that stimulates the translocation of Abl to the nucleus of a cell. The cell can be any of the cells described herein (e.g., see above). Methods for identifying or detecting a cell as expressing MUC1 mRNA or protein are well known to those in the art and are described above. Suitable inhibitory concentrations of the compound can be elucidated through routine experimentation and such optimization is well known to one of skill in the art. As described above, the cell may be co-cultured with one or more additional genotoxic agents.

Methods of detecting the subcellular localization of Abl are well known to those of ordinary skill in the art (see, for example, the Example section below). Generally, the more nuclear localization of Abl in the presence of a compound as compared to the nuclear localization in the absence of the compound indicates that the compound inhibits the nuclear localization of Abl. Nuclear localization of Abl can be detected, for example, by cell fractionation (i.e., detecting the amount of Abl in a cytosolic versus a nuclear extract prepared from the same source of cells) and immunoblotting or ELISA (see, Examples below). Alternatively, localization of Abl can be done in situ, generally by methods including, but not limited to: (i) fixing the cells; (ii) treatment of the fixed cells with detectably-labeled antibodies specific to Abl; and (iii) detecting the signal produced by the detectable label using any of a number of methods known to those in the art, including fluorescence-activated cell sorting (FACS) and confocal microscopy. The detectable label can be conjugated to the first antibody (the primary antibody which specifically recognizes Abl) or on a secondary antibody which is capable of binding to the first antibody. Alternatively, the first antibody can be conjugated to the first member of a binding pair (i.e., strepavidin or biotin) and the second member of the binding pair can be linked to the detectable moiety. The detectable moiety can include radiolabels (e.g., ¹²⁵I, ³⁵S, ³³P, or ³²P), fluorescent labels (e.g., texas red, fluorescein), a luminescent moiety (e.g., a lanthanide), or a one or more members of a FRET pair.

In a preferred embodiment, screening assays (in vivo or in vitro) can be performed in any format that allows for rapid preparation, processing, and analysis of multiple reactions. This can be, for example, in multi-well assay plates (e.g., 96 wells or 386 wells). Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

In Vivo Methods of Stimulating the Translocation of Abl to the Nucleus of a Cell

The disclosure features a method of treating a subject having, or is at risk of developing, a cancer, which includes the steps of: optionally identifying a subject as having, or at risk of developing, a cancer comprising one or more cancer cells expressing MUC1; and delivering to the subject a composition comprising a compound that stimulates the interaction between MUC1 and Abl.

Any of the compounds useful in the methods of stimulating the translocation of Abl (e.g., compounds that inhibit an interaction between MUC1 and Abl, or genotoxic agents (e.g., adriamycin or cisplatin (CDDP))) to the nucleus of a cell can be delivered (e.g., administered) to a subject by any of the in vivo methods described above.

Ex Vivo Methods of Stimulating the Translocation of Abl to the Nucleus of a Cell

An ex vivo strategy can involve transfecting or transducing cells obtained from the subject (or from another subject) with a polynucleotide encoding a polypeptide that stimulates the translocation of Abl to the nucleus of a cell (e.g., a polypeptide that inhibits an interaction between MUC1 and Abl). The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide that stimulates the translocation of Abl to the nucleus of a cell (e.g., a polypeptide that inhibits an interaction between MUC1 and Abl). These methods are known in the art of molecular biology and suitable methods are described above.

Methods of Inhibiting Abl

The disclosure provides in vitro, in vivo, and ex vivo methods of inhibiting Abl. While the invention is not limited by any particular mechanism of action, the binding of MUC1, or a phosphorylated form of MUC1, to Abl (a) sequesters Abl at the cell membrane, and (b) inhibits Abl. Thus, in instances where Abl contributes to the growth of tumor cells (e.g., cells that express BCR-Abl, cells that overexpress Abl, or cells that express mutant, oncogenic forms of Abl) inhibition of Abl can be useful in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, such methods can also be useful in the treatment of cancers.

The term “inhibition of Abl,” as used herein, means inhibition of the kinase activity of Abl, inhibition of the nuclear localization of Abl, inhibition of the activation of Abl (e.g., inhibition of an activating phosphorylation of Abl), or inhibition of the expression (e.g., mRNA or protein expression) of Abl. Expression of Abl can include both Abl mRNA and protein. Cancers treatable by the in vivo and ex vivo methods include lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Cells useful in the methods described herein include, for example, yeast, insect, plant, fish, reptile, and mammalian cells (e.g., mouse, rat, rabbit, guinea pig, dog, cat, pig, horse, goat, cow, whale, monkey, or human). The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines.

Where the methods include the step of identifying a cancer cell as expressing Abl, such identification can include, for example, identifying (or detecting) whether a cell expresses Abl mRNA or MUC1 protein. Suitable methods of identifying (or detecting) the expression of Abl protein or Abl mRNA are well known to those of skill in the art, and are described herein. These methods can include, for example, SDS-polyacrylamide gel electrophoresis/western blotting techniques using antibodies specific for Abl (for detection of protein), or RT-PCR or northern blotting techniques for detection of mRNA expression. The cancer cell can be any cell that expresses Abl, including any cell described above and any cells that express an endogenous Abl. The Abl-expressing cells will generally express a form of Abl that contributes to cancer (e.g., an oncogenic form (e.g., BCR-Abl)) or will express Abl at a level or concentration in the cell that contributes to the growth or viability of the cancer cell.

Compounds useful in the methods of inhibiting Abl include any of the compounds described herein, or any other compounds with the appropriate inhibitory activity. Suitable compounds can include small molecules, antibodies, an antibody fragments, polypeptides, or a peptidomimetics.

In one embodiment of the method, inhibition of Abl is inhibition of Abl kinase activity. Several exemplary compounds for inhibiting the kinase activity of Abl are well known to those in the art and include, for example, Gleevec (imatinib mesylate, also known as STI571; Peggs, et al. (2004) Clin Exp Med. 4(1):1-9; Drucker et al. (2004) Adv Cancer Res. 91:1-30), AMN107 (Weisberg et al. (2006) Br. J. Cancer 94(12):1765-9); and dasatinib (2006) N Engl. J. Med. 354(24):2531-41).

Compounds can also include nucleic acids, for example, nucleic acids that inhibit the mRNA or protein expression of Abl, for example, an antisense oligonucleotide that hybridizes to an Abl mRNA transcript, or an Abl specific small interference RNA (siRNA). Antisense oligonucleotides hybridize to Abl transcripts and have the effect in the cell of inhibiting expression of Abl.

Antisense compounds are generally used to interfere with protein expression either by, for example, interfering directly with translation of a target mRNA molecule, by RNAse-H-mediated degradation of the target mRNA, by interference with 5′ capping of mRNA, by prevention of translation factor binding to the target mRNA by masking of the 5′ cap, or by inhibiting of mRNA polyadenylation. The interference with protein expression arises from the hybridization of the antisense compound with its target mRNA. A specific targeting site on a target mRNA of interest for interaction with a antisense compound is chosen. Thus, for example, for modulation of polyadenylation a preferred target site on an mRNA target is a polyadenylation signal or a polyadenylation site. For diminishing mRNA stability or degradation, destabilizing sequences are preferred target sites. Once one or more target sites have been identified, oligonucleotides are chosen which are sufficiently complementary to the target site (i.e., hybridize sufficiently well under physiological conditions and with sufficient specificity) to give the desired effect.

With respect to this disclosure, the term “oligonucleotide” refers to an oligomer or polymer of RNA, DNA, a combination of the two, or a mimetic of either. The term includes oligonucleotides composed of naturally-occurring nucleobases, sugars, and covalent internucleoside (backbone) linkages. The normal linkage or backbone of RNA and DNA is a 3′ to 5′ phosphodiester bond. The term also refers however to oligonucleotides composed entirely of, or having portions containing, non-naturally occurring components which function in a similar manner to the oligonucleotides containing only naturally-occurring components. Such modified substituted oligonucleotides are often preferred over native forms because of desirable properties such as, for example, enhanced cellular uptake, enhanced affinity for target sequence, and increased stability in the presence of nucleases. In the mimetics, the core base (pyrimidine or purine) structure is generally preserved but (1) the sugars are either modified or replaced with other components and/or (2) the inter-nucleobase linkages are modified. One class of nucleic acid mimetic that has proven to be very useful is referred to as protein nucleic acid (PNA). In PNA molecules the sugar backbone is replaced with an amide-containing backbone, in particular an aminoethylglycine backbone. The bases are retained and are bound directly to the aza nitrogen atoms of the amide portion of the backbone. PNA and other mimetics useful in the instant disclosure are described in detail in U.S. Pat. No. 6,210,289, the disclosure of which is incorporated herein by reference in its entirety.

The antisense oligomers to be used in the methods described herein generally comprise about 8 to about 100 (e.g., about 14 to about 80 or about 14 to about 35) nucleobases (or nucleosides where the nucleobases are naturally occurring).

The antisense oligonucleotides can themselves be introduced into a cell or an expression vector containing a nucleic sequence (operably linked to a TRE) encoding the antisense oligonucleotide can be introduced into the cell. In the latter case, the oligonucleotide produced by the expression vector is an RNA oligonucleotide and the RNA oligonucleotide will be composed entirely of naturally occurring components.

Also useful in the method of inhibiting the expression of Abl are double-stranded small interference RNA (siRNA) homologous to Abl DNA, which can be used to reduce expression of Abl in a cell. See, e.g., Fire et al. (1998) Nature 391:806-811; Romano and Masino (1992) Mol. Microbiol. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017-1026. The disclosures of all these articles are incorporated herein by reference in their entirety.

The sense and anti-sense RNA strands of siRNA can be individually constructed using chemical synthesis and enzymatic ligation reactions using procedures known in the art. For example, each strand can be chemically synthesized using naturally occurring nucleotides or variously modified nucleotides designed to increase the biological stability of the molecule or to increase the physical stability of the duplex formed between the sense and anti-sense strands, e.g., phosphorothioate derivatives and acridine substituted nucleotides. Some of the nucleotides (e.g., the terminal (either terminus) one, two, three, or four nucleotides) can also be deoxyribonucleotides. The sense or anti-sense strand can also be produced biologically using an expression vector into which a target Abl sequence (full-length or a fragment) has been subcloned in a sense or anti-sense orientation. The sense and anti-sense RNA strands can be annealed in vitro before delivery of the dsRNA to cells. Alternatively, annealing can occur in vivo after the sense and anti-sense strands are sequentially delivered to cells.

The subjects may be further treated with (e.g., be exposed to, have delivered, or have administered) one or more additional genotoxic or chemotherapeutic agents. Such genotoxic or chemotherapeutic agents can include any of the genotoxic or chemotherapeutic agents described herein. Subjects may also be further treated with one or more kinase inhibitors (e.g., antibodies that inhibit receptor tyrosine kinases (e.g., Trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Inhibiting Abl

The disclosure provides an in vitro method of inhibiting Abl. The method includes the steps of: optionally identifying a cell as expressing MUC1, and culturing a cell with a compound that inhibits Abl. Such methods can have general applicability in scientific studies on the role of MUC1 in the Abl signal transduction pathway. These methods may also be useful in any studies where inhibition of Abl is advantageous. Furthermore, as above, such in vitro methods of inhibiting Abl can be used as secondary assays in screening cascades in the pursuit of inhibitors of the MUC1-Abl regulatory axis.

Methods for identifying or detecting a cell as expressing MUC1 mRNA or protein are well known to those in the art and are described above. Methods for culturing a cell with an inhibitor are widely known in the art, described in the Examples section below, and also described above. Suitable concentrations of the inhibitory compound can be elucidated through routine experimentation and such optimization is well known to one of skill in the art. As described above, the cell may be co-cultured with one or more additional genotoxic or chemotherapeutic agents.

Suitable methods of determining inhibition of Abl expression, or Abl localization are described herein (see above).

Methods of determining the inhibition of kinase activity are well known to those of skill in the art. Inhibition of the kinase activity of Abl can be measured by monitoring the phosphorylation state of endogenous, natural Abl substrates (e.g., MUC1), examples of which are described above and in the Examples section (see also, e.g., de Jong et al. (1997) Oncogene 14(5):507-513). The phosphorylation state of these substrates can be measured in intact cells using antibody-mediated immunofluorescence or immunohistochemical techniques. The phosphorylation state of endogenous substrates can alternatively be measured by solubilizing the cells in Laemmli buffer and subjecting the solubilized extracts to SDS-PAGE, followed by western blotting with antibodies specific for phosphorylated residues in the Abl substrate proteins (e.g., an antibody that specifically recognizes a phosphorylated amino acid residue in MUC1 or the MUC1-CD). Alternatively, antibodies that recognized non-phosphorylated Abl substrates (e.g., non-phosphorylated MUC1) can be amenable for this assay as they can be used to detect changes in protein mobility consistent with protein modification (e.g., phosphorylation).

In Vivo Methods of Inhibiting Abl

The disclosure features an in vivo method of inhibiting Abl, which includes the steps of optionally identifying a subject as having, or at risk of developing, a cancer containing one or more cells expressing Abl, and delivering to the subject a compound containing a fragment of MUC1 capable of binding to and/or inhibiting Abl.

The disclosure also features an in vivo method of inhibiting Abl, which includes the steps of optionally identifying a subject as having, or at risk of developing, an inflammatory disorder mediated by one or more immune cells expressing Abl, and delivering to the subject a compound containing a fragment of MUC1 capable of binding to and/or inhibiting Abl.

In one in vivo approach, a compound that inhibits Abl is administered to a subject (e.g., a human subject (e.g., a human patient)). Methods of administration useful in the method are described above.

Ex Vivo Methods of Inhibiting Abl

An ex vivo strategy can involve transfecting or transducing cells obtained from the subject (or from another subject) with a polynucleotide encoding a polypeptide fragment of MUC1 that is capable of binding to and/or inhibiting Abl. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide fragment of MUC1 that is capable of binding to and/or inhibiting Abl. These methods are known in the art of molecular biology and suitable methods are described above.

Methods of Inhibiting BCR-Abl

The disclosure also provides in vitro, in vivo, and ex vivo methods of inhibiting BCR-Abl. As used herein, the term “inhibition of BCR-Abl” means inhibition of the kinase activity of BCR-Abl or inhibition of the expression (e.g., mRNA or protein expression) of BCR-Abl.

The discovery of the Philadelphia chromosome (Ph), an abnormally short chromosome 22 that is one of the two chromosomes involved in a translocation (an exchange of material) with chromosome 9, was a landmark. The discovery led to the identification in Chronic Myelogenous Leukemia (CML) cells of the BCR-Abl fusion gene and its corresponding protein. Although the exact breakage and fusion points can vary, generally the BCR-Abl gene is a 210 kDa chimeric protein with the first 900-930 amino acids of the BCR polypeptide and exists mainly as a cytoplasmic protein. The BCR-ABL gene encodes a protein with deregulated (uncontrolled) tyrosine kinase activity. The presence of this protein in the CML cells is strong evidence of its pathogenetic (disease-causing) role. The efficacy in CML of a drug that inhibits the BCR-ABL tyrosine kinase has provided the final proof that the BCR-ABL oncoprotein is the unique cause of CML. The Ph chromosome is also found in a form of acute lymphoblastic leukemia (ALL). While not limited by any particular mechanism of action, it appears that the binding of MUC1, or a phosphorylated form of MUC1, to the SH2 domain of Abl (a) sequesters Abl at the cell membrane, and (b) inhibits Abl. BCR-Abl also contains the SH2 domain of Abl kinase. Thus, in cancers or cancer cells where BCR-Abl is expressed and/or contributes to the growth of tumor cells, the methods of inhibition of BCR-Abl can be useful in inhibiting the growth of a cancer cell. Inhibition of cell growth can be a reversible inhibition of cell growth, or more preferably can be an irreversible inhibition of cell growth (i.e., causing the death of the cell). Where the methods are in vivo or ex vivo, such methods can also be useful in the treatment of cancers.

Expression of BCR-Abl includes both BCR-Abl mRNA and BCR-Abl protein expression. Cancers treatable by the in vivo and ex vivo methods include, but are not limited to, hematological cancers such as B cell lymphomas. Cells useful in the methods described herein include, for example, yeast, insect, plant, fish, reptile, and mammalian cells (e.g., mouse, rat, rabbit, guinea pig, dog, cat, pig, horse, goat, cow, whale, monkey, or human) or any cell that expresses an endogenous or exogenous form of BCR-Abl. The cells can be normal or malignant and of any histological type, e.g., without limitation, epithelial cells, fibroblasts, lymphoid cells, macrophages/monocytes, granulocytes, keratinocytes, or muscle cells. Cancer cells useful in the method can include cancer cells from cancers such as, but not limited to, lung cancer, breast cancer, colon cancer, pancreatic cancer, renal cancer, stomach cancer, liver cancer, bone cancer, hematological cancer (e.g., B cell lymphomas), neural tissue cancer, melanoma, thyroid cancer, ovarian cancer, testicular cancer, prostate cancer, cervical cancer, vaginal cancer, and bladder cancer. Suitable cell lines include those recited in the examples, e.g., breast cancer or colon cancer cell lines.

Where the methods include the step of identifying a cancer cell as expressing BCR-Abl, such identification can include, for example, identifying (or detecting) whether a cell expresses BCR-Abl mRNA or BCR-Abl protein. Suitable methods of identifying (or detecting) the expression of BCR-Abl protein or BCR-Abl mRNA are well known to those of skill in the art, and are described herein. These methods can include, for example, SDS-polyacrylamide gel electrophoresis/western blotting techniques using antibodies specific for Abl (for detection of protein), or RT-PCR or northern blotting techniques for detection of mRNA expression.

Compounds useful in the methods of inhibiting BCR-Abl include any of the compounds described herein, or any other compounds with the appropriate inhibitory activity. Suitable compounds can include small molecules, antibodies, an antibody fragments, polypeptides, or a peptidomimetics.

In one embodiment of the method, inhibition of BCR-Abl is inhibition of the Abl kinase activity associated with BCR-Abl. Several exemplary compounds for inhibiting the kinase activity of BCR-Abl are well known to those in the art and include, for example, Gleevec (imatinib mesylate, also known as STI571; Peggs, et al. (2004) Clin Exp Med. 4(1):1-9; Drucker et al. (2004) Adv Cancer Res. 91:1-30), AMN107 (Weisberg et al. (2006) Br. J. Cancer 94(12):1765-9); and dasatinib (2006) N Engl. J. Med. 354(24):2531-41).

Compounds can also include nucleic acids, for example, nucleic acids that inhibit the mRNA or protein expression of BCR-Abl, for example, an antisense oligonucleotide that hybridizes to a BCR-Abl mRNA transcript, or a BCR-Abl specific small interference RNA (siRNA). Antisense oligonucleotides and siRNAs hybridize to Abl transcripts and have the effect in the cell of inhibiting expression of BCR-Abl.

The subjects may be further treated with (e.g., be exposed to, have delivered, or have administered) one or more additional genotoxic or chemotherapeutic agents. Such genotoxic or chemotherapeutic agents can include any of the genotoxic or chemotherapeutic agents described herein. Subjects may also be further treated with one or more kinase inhibitors (e.g., antibodies that inhibit receptor tyrosine kinases (e.g., trastuzumab (e.g., Herceptin®)), or small molecules that inhibit kinases (e.g., gefitinib (e.g., Iressa®), erlotinib (e.g., Tarceva®), imatinib mesylate (e.g., Gleevec®), or sunitinib malate (e.g., Sutent®)).

In Vitro Methods of Inhibiting BCR-Abl

The disclosure provides an in vitro method of inhibiting BCR-Abl. The method includes the steps of: optionally identifying a cell as expressing MUC1, and culturing a cell with a compound that inhibits BCR-Abl. Such methods can have general applicability in scientific studies on the role of MUC1 in the Abl signal transduction pathway. These methods may also be useful in any studies where inhibition of BCR-Abl is advantageous. Furthermore, as above, such in vitro methods of inhibiting BCR-Abl can be used as secondary assays in screening cascades in the pursuit of inhibitors of the MUC1-BCR-Abl regulatory axis.

Methods for identifying or detecting a cell as expressing MUC1 mRNA or protein are well known to those in the art and are described above. Methods for culturing a cell with an inhibitor are widely known in the art, described in the Examples section below, and also described above. Suitable concentrations of the inhibitory compound can be elucidated through routine experimentation and such optimization is well known to one of skill in the art. As described above, the cell may be co-cultured with one or more additional genotoxic or chemotherapeutic agents.

Suitable methods of determining inhibition of BCR-Abl kinase activity or BCR-Abl expression are described herein (see “Inhibition of Abl”).

In Vivo Methods of Inhibiting BCR-Abl

The disclosure also features an in vivo method of inhibiting BCR-Abl (B-cell Receptor-Abl fusion), which includes the step of: optionally identifying a subject as having, or at risk of developing, a cancer containing one or more cells expressing BCR-Abl, and delivering to the subject a compound containing a fragment of MUC1 capable of binding to or inhibiting BCR-Abl.

Any of the compounds that inhibits BCR-Abl described herein can be administered to a subject as described above in preceeding sections.

Ex Vivo Methods of Inhibiting BCR-Abl

An ex vivo strategy can involve transfecting or transducing cells obtained from the subject (or from another subject) with a polynucleotide encoding a polypeptide that inhibits BCR-Abl. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide that inhibits BCR-Abl. These methods are known in the art of molecular biology and suitable methods are described above.

Methods of Treating a Neurologic Disorder

Abl kinase activity is associated with the phosphorylation of Tau, a biochemical event linked, for example, to increased risk and incidence of Alzheimer's disease. Since MUC1 binds to and inhibits Abl, use of MUC or Abl-binding fragments of MUC1 may be useful to treat certain neurologic disorders. Such neurologic disorders include, but are not limited to: Parkinson's disease, Alzheimer's Disease or any other tauopathy (e.g., Pick's disease, progressive supranuclear palsy (PSP), frontotemporal dementia (FTD), or corticobasal degeneration (CBD)). Thus, provided herein are methods of inhibiting a neurologic disorder, which include the steps of: (a) identifying a subject as having, suspected of having, or at risk of developing, a neurologic disorder, where one of more of the cells involved in the neurologic disorder express Abl, and delivering to the subject a compound containing a fragment of MUC1 capable of binding to and/or inhibiting Abl kinase.

In Vivo Methods of Treating a Neurologic Disorder

The disclosure also features an in vivo method of treating a neurologic disorder, which includes the step of optionally identifying a subject as having, suspected of having, or at risk of developing, a neurologic disorder, where one or more neuronal cells involved in the neurologic disorder express Abl, and delivering to the subject a compound containing a fragment of MUC1 capable of binding to or inhibiting Abl. Inhibition of Abl is described above.

Where the method includes the step of identifying a neuronal cell involved in a neurologic disorder as expressing Abl, such identification can include, for example, identifying (or detecting) whether a cell expresses Abl mRNA or Abl protein. Suitable methods of identifying (or detecting) the expression of Abl protein or Abl mRNA are well known to those of skill in the art, and are described herein. These methods can include, for example, SDS-polyacrylamide gel electrophoresis/western blotting techniques using antibodies specific for Abl (for detection of protein), or RT-PCR or northern blotting techniques for detection of mRNA expression. The method can also optionally involve identifying the neuronal cell involved in the neurologic disorder as expressing MUC1. Expression of MUC1 also includes both MUC1 mRNA and protein, and can be readily detected by any of the methods described above.

Compounds useful in the methods of treating a neurologic disorder (compounds that inhibit Abl) include fragments of MUC1 capable of binding to and/or inhibiting Abl, any other of the compounds described herein, or any other compounds with the appropriate inhibitory activity. Suitable compounds can include small molecules, antibodies, an antibody fragments, polypeptides, or a peptidomimetics.

In one embodiment of the method, inhibition of Abl is inhibition of Abl kinase activity. Several exemplary compounds for inhibiting the kinase activity of Abl are well known to those in the art and include, for example, Gleevec (imatinib mesylate, also known as STI571; Peggs, et al. (2004) Clin Exp Med. 4(1):1-9; Drucker et al. (2004) Adv Cancer Res. 91:1-30), AMN107 (Weisberg et al. (2006) Br. J. Cancer 94(12):1765-9); and dasatinib (2006) N Engl. J. Med. 354(24):2531-41).

Compounds can also include nucleic acids, for example, nucleic acids that inhibit the mRNA or protein expression of Abl, for example, an antisense oligonucleotide that hybridizes to a Abl mRNA transcript, or a Abl specific small interference RNA (siRNA). Antisense oligonucleotides hybridize to Abl transcripts and have the effect in the cell of inhibiting expression of Abl (as described above).

Any of the compounds described for use in the vivo methods may be administered to a subject as described above. The compounds can be further administered to a subject with one or more additional therapies useful in the treatment of neurologic disorders including, for example, tacrine (Cognex®), donepezil (Aricept®), rivastigmine (Exelon®), galantamine (Razadyne®), or vitamin E.

Ex Vivo Methods of Treating a Neurologic Disorder

An ex vivo strategy can involve transfecting or transducing cells obtained from the subject (or from another subject) with a polynucleotide encoding a polypeptide fragment of MUC1 capable of binding to or inhibiting Abl. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide fragment of MUC1 capable of binding to or inhibiting Abl. These methods are known in the art of molecular biology and suitable methods are described above.

Methods of Treating an Diabetes-Associated Atherosclerotic Lesion

Provided herein are in vivo and ex vivo methods of treating an atherosclerotic lesion (e.g, a diabetes-associated atherosclerotic lesion). The method includes the steps of optionally identifying a subject having, or at risk of developing, a diabetes-associated atherosclerotic lesion, wherein the atherosclerotic lesion contains one or more cells expressing Abl, and delivering to the subject a compound comprising a MUC1 fragment capable of binding to and/or inhibiting Abl.

Diabetes is associated with accelerated atherosclerosis, the major factor contributing to increased mortality and morbidity in the diabetic population. Abl kinase inhibitor Imatinib was shown to prevent the development of atherosclerotic lesions and diabetes-induced inflammatory cytokine overexpression in the aorta (Lassila et al. (2004) Arterioscler. Thromb Vasc Biol. 24(5):935-42). Since the binding of MUC1, or a phosphorylated form of MUC1, to Abl (a) sequesters Abl at the cell membrane, and (b) inhibits Abl, such methods of inhibition can have general applicability in treating atherosclerosis.

The methods optionally include a step of identifying a cell (e.g., a vascular epithelial cell, an aortic muscle cell, or an immune cell mediating inflammation at an atherosclerotic site) as expressing Abl and/or MUC1. Such identification can include, for example, identifying (or detecting) whether a cell expresses Abl mRNA or Abl protein. Suitable methods of identifying (or detecting) the expression of Abl protein or Abl mRNA are well known to those of skill in the art, and are described herein. Compounds useful in the methods of treating an atherosclerotic lesion can include fragments of MUC1 capable of binding to and/or inhibiting Abl, any of the compounds described herein, or any other compounds with the appropriate inhibitory activity. Suitable compounds can be small molecules, antibodies, an antibody fragments, polypeptides, or a peptidomimetics. Compounds can also include nucleic acids, for example, nucleic acids that inhibit the mRNA or protein expression of Abl (e.g., siRNA or anti-sense nucleic acids; see “Methods of Inhibiting Abl”). Other exemplary compounds for use in the methods include MUC1 or Abl polypeptides or their functional fragments. Examples of functional fragments of MUC1 include, for example, the MUC1-CD (SEQ ID NO:2) or MUC1-CD-YTNP (SEQ ID NO:3), or fragments of the MUC1-CD that contain pYTNP (e.g., a polypeptide containing amino acids 2-71, amino acids 5-70, amino acids 10-70, amino acids 10-65, amino acids 15-70, amino acids 20-70, amino acids 25-70, amino acids 30-70, amino acids 35-70, amino acids 40-70, amino acids 45-70, amino acids 50-70, amino acids 55-70, or amino acids 55-70). Examples of functional fragments of Abl polypeptide include all or part of the SH2 domain of c-Abl (SEQ ID NO:5) or all or part of the SH3 domain of c-Abl (SEQ ID NO:8).

In Vivo Methods of Treating an Atherosclerotic Lesion

In one in vivo approach, a compound used to treat an atherosclerotic lesion is administered to a subject. The subject can be any mammalian subject described herein. Any compound useful in treating an atherosclerotic lesion described herein or with appropriate activity can be delivered (e.g., administered) to a subject by any method described above.

Ex Vivo Methods of Treating an Atherosclerotic Lesion

An ex vivo strategy can involve transfecting or transducing cells obtained from the subject (or from another subject) with a polynucleotide encoding a polypeptide fragment of MUC1 capable of binding to and/or inhibiting Abl. The transfected or transduced cells are then administered to the subject. The cells can be any of a wide range of types including, without limitation, any of the cells described above. The ex vivo methods include the steps of harvesting cells from a subject, culturing the cells, transducing them with an expression vector, and maintaining the cells under conditions suitable for expression of the polypeptide fragment of MUC1 capable of binding to and/or inhibiting Abl. These methods are known in the art of molecular biology and suitable methods are described above.

The following examples are intended to illustrate, not limit, the invention.

### EXAMPLES

### Example 1

### Materials and Methods

Cell culture. Human HeLa cervical cancer cells, HCT116/vector (HCT116 colorectal carcinoma cells stably transfected with an empty vector), HCT116/MUC1 (HCT116 colorectal carcinoma cells stably transfected with an expression vector encoding a MUC1 polypeptide), HCT116/MUC1(Y46F) (HCT116 colorectal carcinoma cells stably transfected with an expression vector encoding a mutant MUC1 polypeptide containing an amino acid substitution at position 46 changing a tyrosine (Y) to phenylalanine (F)) colon cancer cells (Ren et al., (2002) J. Biol. Chem. 277: 17616-17622) and 293 cells were cultured in Dulbecco's modified medium with 10% heat-inactivated fetal bovine serum, 100 units/ml penicillin, 100 μg/ml streptomycin and 2 mM L-glutamine. Human ZR-75-1/vector (ZR-75-1 breast cancer cells stably transfected with an empty vector) and ZR-75-1/MUC1siRNA (ZR-75-1 breast cancer cells stably transfected with an expression vector encoding an siRNA specific for MUC1) breast cancer cells (Ren et al., (2004) Cancer Cell 5:163-175) were grown in RPMI1640 medium containing 10% heat-inactivated fetal bovine serum and antibiotics. Cells were treated with 1 μM adriamycin (ADR; Calbiochem), 100 μM cisplatin (CDDP; Sigma) or 10 μM STI571 (Gleevec; Novartis, Basel, Switzerland).

Cell transfections. HeLa cells were stably transfected with pCMV or pCMV-MUC1 using LipofectAMINE and selected in the presence of the antibiotic G418 (neomycin). The pIRESpuro2-MUC1(Y60F) and GFP-c-Abl(R152L) vectors were constructed by site-directed mutagenesis as described (Li et al., (2001) J. Biol. Chem. 276:6061-6064). HCT116 cells were stably transfected with pIRESpuro2-MUC1(Y60F) using LipofectAMINE and selected in the presence of puromycin (Calbiochem-Novabiochem Co., San Diego, Calif.). 293 cells were transiently transfected with pIRESpuro2, pIRESpuro2-MUC1, pIRESpuro2-MUC1(Y60F), GFP, GFP-c-Abl, GFP-c-AblΔNES and GFP-c-Abl(R152L) using LipofectAMINE. For downregulation of c-Abl, HCT116/vector, HCT116/MUC1 and HCT116/MUC1(Y60F) cells were seeded (5×10⁵/well) on 6-well plates. After 24 h, the cells were transfected with 1 nM SMART pool c-Abl siRNAs or non-specific control pool siRNAs (Upstate Biotechnology Inc., Charlottesville, Va.) using LipofectAMINE. The cells were incubated for an additional 72 h and then harvested for analysis.

Subcellular fractionation. Nuclear fractions were prepared as described (Kharbanda et al., (1996) Cancer Res. 56:3617-3621). Cytosolic fractions were prepared as described (Hill et al., (2004) EMBO J. 23:2134-2145). Cell membrane fractions were prepared with the Plasma Membrane Extraction Kit (BioVision Research Products, Mountain View, Calif.). Mitochondria were purified as described (Ren et al., (2004) Cancer Cell 5:163-175; Ren et al., (2006) Oncogene 25:20-31).

Immunoprecipitations and immunoblot analysis. Cell lysates were prepared as described (Kharbanda et al., (1995) Nature 376:785-788); Yoshida et al., (2005) Nat. Cell Biol. 7:278-285). Soluble proteins were incubated with anti-MUC1-N (Kufe et al., (1984) Hybridoma 3:223-232), anti-MUC1-C (Ab5; Neomarker, Montreal, Quebec), anti-Flag (Sigma, St. Louis, Mo.), anti-c-Abl (Ab3; Oncogene, San Diego, Calif.) or anti-14-3-3 (Abcam Inc., Cambridge, Mass.) antibodies for 2 hours at 4° C., followed by precipitation with protein G-Sepharose beads for 1 hour. Immune complexes and cell lysates (50 μg) were subjected to immunoblot analysis with anti-MUC1-N, anti-MUC1-C, anti-c-Abl (Calbiochem, San Diego, Calif.), anti-lamin B (Oncogene, San Diego, Calif.), anti-IκBα (Santa Cruz Biotechnology, Santa Cruz, Calif.), anti-GFP (BD Biosciences, San Jose, Calif.), anti-Flag (Sigma, St. Louis, Mass.), anti-β-actin (Sigma, St. Louis, Mo.), anti-phospho-c-Abl-Thr-735 (Cell Signaling Technology), anti-14-3-3 (Santa Cruz Biotechnology, Santa Cruz, Calif.), anti-PKC5 (Santa Cruz Biotechnology), anti-HSP60 (BD Biosciences, San Jose, Calif.), anti-PCNA (Oncogene), or anti-calnexin (Stressgen, San Diego, Calif.) antibodies. The antigen-antibody complexes were visualized by chemiluminescence (NEN Life Science Products, Wellesley, Mass.). Intensity of the signals was determined by densitometric scanning. Statistical significance was determined by the Student's t-test.

In vitro kinase assays. GST-c-Abl or GST-c-Abl(K-R) (kinase defective mutant c-Abl polypeptide containing an amino acid substitution of lysine to arginine in the active site of the kinase) (fusion proteins consisting of glutathione-S-transferase fused in frame with the indicated c-Abl proteins) purified from baculovirus infected Sf9 cells (Yuan et al., (1999) Nature 399:814-817) were incubated in kinase buffer (50 mM HEPES, pH 7.4, 10 mM MgCl₂, 10 mM MnCl₂, 2 mM dithiothreitol, 0.1 mM sodium vanadate) with thrombin-cleaved and purified MUC1-CD (cytoplasmic domain) or MUC1-CD(Y60F) (CD mutant containing an amino acid substitution at position 60 of the CD from tyrosine to phenylalanine) and [γ-³²P]ATP (NEN Life Science Products, Wellesley, Mass.) for 15 minutes at 30° C. Reaction products were analyzed by SDS-polyacrylamide gel electrophoresis (PAGE) and autoradiography.

Immunofluorescence microscopy. Cells cultured on coverslips were fixed in 3.7% formaldehyde, washed with PBS, permeabilized in PBS containing 0.2% Triton X-100 and then postfixed in 3.7% formaldehyde. The cells were blocked with 10% goat serum and stained with anti-MUC1-C antibodies, followed by fluorescein isothiocyanate (FITC)-conjugated secondary (anti-IgG) antibodies. The cells were then incubated with anti-c-Abl (24-11; Santa Cruz Biotechnology, Santa Cruz, Calif.) antibodies, followed by incubation with a Texas Red-goat anti-mouse IgG conjugate (Jackson ImmunoReasearch Laboratory). Nuclei were stained with 2 μM TO-PRO3 (Molecular Probes, Carlsbad, Calif.). Images were captured with a Zeiss LSM510 confocal microscope at 1024×1024 resolution.

Analysis of c-Abl kinase activity. Nuclear lysates were immunoprecipitated with anti-c-Abl (K-12) antibodies as described (Kharbanda et al., (1997) Nature 386:732-735). The precipitates were resuspended in kinase buffer (20 mM HEPES, pH 7.4, 10 mM MgCl₂) containing [γ-³²P]ATP, GST-Crk(120-225) for 20 minutes at 30° C. The reaction products were analyzed by SDS-PAGE and autoradiography.

Binding studies. Purified MUC1-CD and MUC1-CD(Y60F) were incubated in the absence and presence of recombinant truncated Abl (kinase domain; New England Biolabs, Ipswich, Mass.) and 200 μM ATP for 30 minutes at 30° C. GST-c-Abl SH2 (Yoshida et al., 2002) or GST-c-Abl SH2(R152L) bound to glutathione beads was then added for 1 hour at 4° C. After washing, the precipitated proteins were subjected to immunoblot analysis.

Assessment of apoptosis. DNA content was assessed by staining ethanol-fixed cells with propidium iodide (PI) and monitoring by FACScan (Becton Dickinson, Franklin Lakes, N.J.). The percentage of cells with sub-G1 DNA was determined by the MODFIT LT Program (Verity Software, Topsham, Mass.). Statistical significance was determined using the unpaired students t-test and GraftPad software.

### Example 2

### MUC1 attenuates DNA Damage-Induced Targeting of c-Abl to the Nucleus

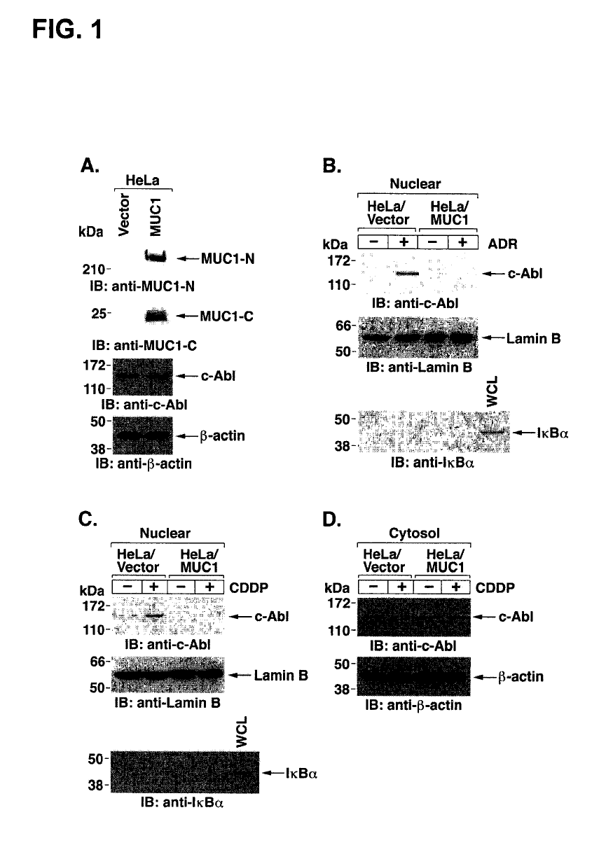

To investigate whether MUC1 regulates nuclear targeting of c-Abl, HeLa cells were stably transfected with an empty expression vector (HeLa/vector) or an expression vector encoding a MUC1 polypeptide (MUC1, MUC1-N-terminal (MUC1-N), or MUC1-C-terminal (MUC1-C)) (HeLa/MUC1). The MUC1 N-terminal (MUC1-N) and C-terminal (MUC1-C) subunits were detectable in HeLa/MUC1, but not HeLa/vector, cells (FIG. 1A). Moreover, c-Abl levels were similar in the absence and presence of MUC1 (FIG. 1A). Consistent with previous results (Yoshida et al., (2005) Nat. Cell. Biol. 7:278-285), treatment of HeLa/vector cells with the DNA damaging agent adriamycin (ADR) was associated with targeting of c-Abl to the nucleus (FIG. 1B). In contrast, there was little to no nuclear targeting of c-Abl in the ADR-treated HeLa/MUC1 cells (FIG. 1B). To verify equal loading and the quality of the nuclear fractionation, the nuclear fractions were immunoblotted with antibodies specific for the nuclear lamin B and cytoplasmic IκBα proteins (FIG. 1B). Treatment of HeLa/vector and HeLa/MUC1 cells with cisplatin (CDDP) confirmed that MUC1 attenuates nuclear targeting of c-Abl by genotoxic anti-cancer agents (FIG. 1C). Similar to the results using ADR, CDDP treatment was associated with a decrease in cytosolic c-Abl in HeLa/vector, but not HeLa/MUC1, cells (FIG. 1D).

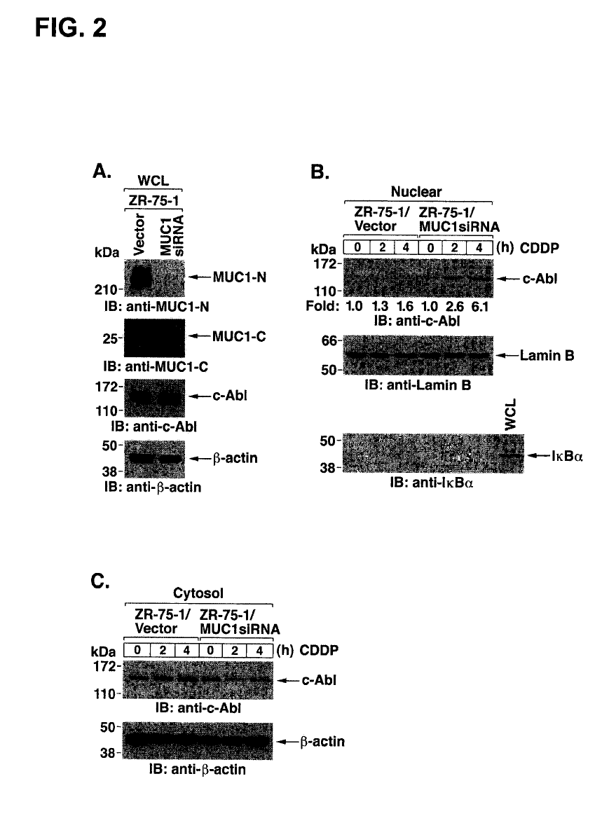

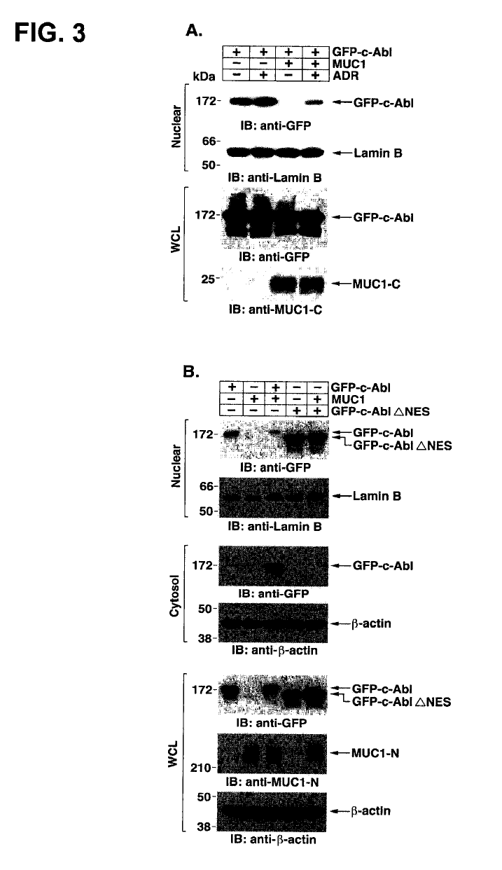

Endogenous MUC1 expression can be knocked-down with a MUC1-specific siRNA (MUC1siRNA) in human ZR-75-1 breast cancer cells, and stable cells have been generated in which ZR-75-1 cells stably express the MUC1siRNA (ZR-75-1/MUC1siRNA) or that contain only the empty expression vector (ZR-75-1/vector). Silencing MUC1 in the ZR-75-1 cells had no effect on c-Abl levels (FIG. 2A). When ZR-75-1/vector cells were treated with CDDP, there was no detectable targeting of c-Abl to the nucleus (FIG. 2B). However, in ZR-75-1/MUC1siRNA cells silenced for MUC1, nuclear targeting of c-Abl was increased about 6-fold in response to CDDP treatment (FIG. 2B). Cytosolic c-Abl was also decreased by CDDP treatment of ZR-75-1/MUC1siRNA, but not ZR-75-1/vector, cells (FIG. 2C). To further analyze the effects of MUC1 on c-Abl localization, transient overexpression of GFP-tagged c-Abl in 293 cells resulted in targeting of c-Abl to the nucleus that was detectable constitutively and increased by ADR treatment (FIG. 3A). By contrast, constitutive and ADR-induced nuclear targeting of c-Abl was attenuated by coexpression of MUC1 (FIG. 3B). Similar results were obtained when the transfected cells were treated with CDDP. The c-Abl mutant with deletion of the nuclear export signal (c-AblΔNES) localizes exclusively in the nucleus (Yoshida et al., (2005) Nat. Cell. Biol. 7:278-285). Importantly, MUC1 expression had little effect on nuclear localization of GFP-c-AblΔNES (FIG. 3B).

### Example 3

### MUC1 Associates with c-Abl at the Cell Membrane

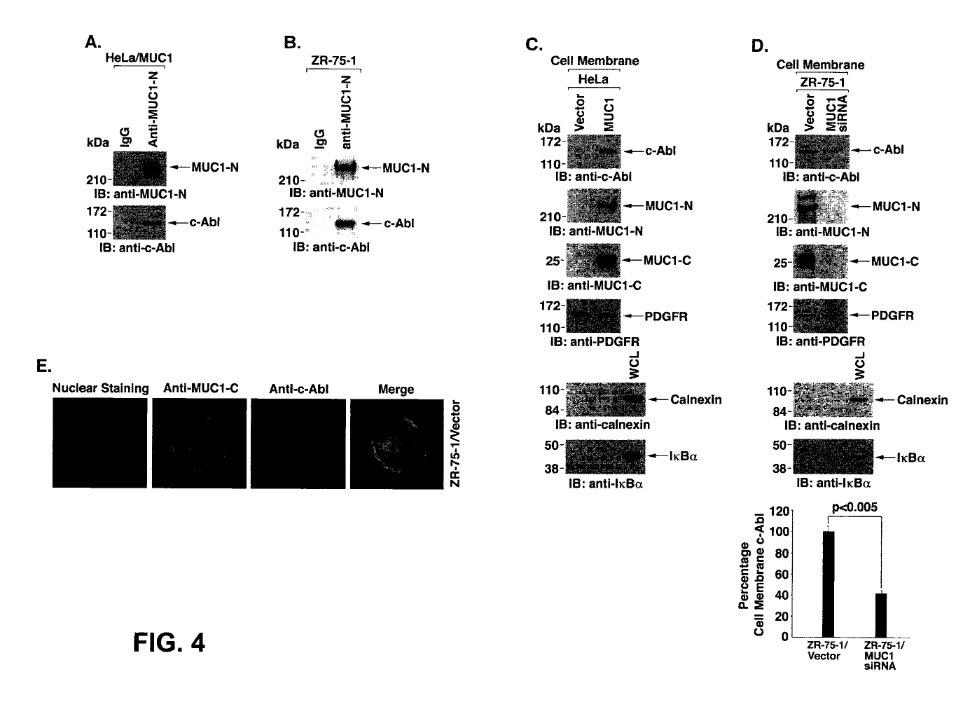

To determine if MUC1 associates with c-Abl, lysates from HeLa/MUC1 cells were precipitated with anti-MUC1-N antibodies or, as a control, non-specific immunoglobulin (IgG). Immunoblot analysis of the precipitates with anti-c-Abl demonstrated that MUC1 associates with c-Abl (FIG. 4A). Communoprecipitation studies performed on ZR-75-1 cells confirmed that MUC1 forms a complex with c-Abl (FIG. 4B). MUC1 is expressed at the cell membrane as a heterodimer of MUC1-N and MUC1-C. Consequently, an experiment was designed to test if c-Abl localizes to the cell membrane as a function of MUC1 expression. Immunoblot analysis showed a substantially higher level of c-Abl in HeLa/MUC1 cell membranes as compared to those purified from HeLa/vector cells (FIG. 4C). Equal loading and purity of the cell membrane fraction was confirmed by immunoblotting with antibodies specific for the cell membrane-associated platelet-derived growth factor receptor (PDGF-R), endoplasmic reticulum-associated calnexin, and cytosolic IκBα proteins (FIG. 4C). Consistent with the results in HeLa stable cell lines, localization of c-Abl to the cell membrane was significantly decreased, but not completely abrogated, by silencing MUC1 in ZR-75-1/MUC1siRNA cells (FIG. 4D). Confocal microscopy further showed colocalization of MUC1 and c-Abl at the cell membrane (FIG. 4E). These findings indicate that c-Abl associates with MUC1 at the cell membrane.

### Example 4

### c-Abl Phosphorylates the MUC1 Cytoplasmic Domain

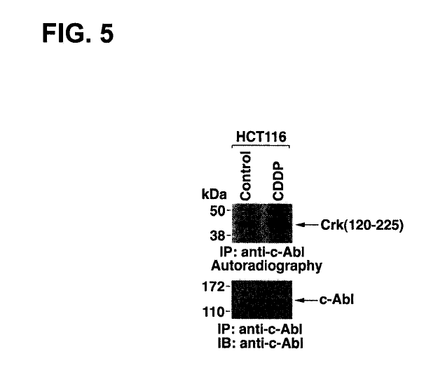

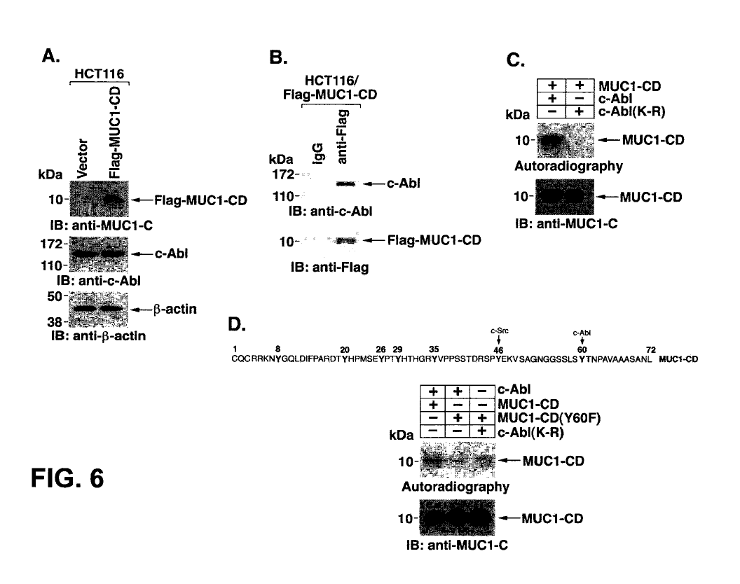

To identify the region of MUC1 that associates with c-Abl, studies were performed on HCT116 colon cancer cells. As found in other cell types, treatment of the HCT116 cells with CDDP was associated with induction of c-Abl activity (FIG. 5). Consequently, HCT116 cells that stably express Flag or a Flag-tagged MUC1 cytoplasmic domain (MUC1-CD) (Huang et al., (2003) Cancer Biol. Ther. 2:702-706) (FIG. 6A) were analyzed. Expression of Flag-MUC1-CD had no detectable effect on c-Abl levels (FIG. 6A). Immunoblot analysis of anti-Flag precipitates with antibodies specific for c-Abl further showed that MUC1-CD is sufficient for the association with c-Abl (FIG. 6B). To determine if MUC1-CD is a substrate for c-Abl, purified kinase-active c-Abl and MUC1-CD were incubated in the presence of [γ-³²P]ATP. Analysis of the reaction products by SDS-PAGE and autoradiography showed phosphorylation of MUC1-CD (FIG. 6C). There was no detectable phosphorylation when MUC1-CD was incubated with a purified kinase-inactive c-Abl(K-R) (Kharbanda et al., (1995) J. Biol. Chem. 270:30278-30281) (FIG. 6C). MUC1-CD contains a consensus c-Abl phosphorylation site (YXXP) at Y⁶⁰TNP (FIG. 6D). Consequently, an experiment was performed to test if c-Abl phosphorylation of MUC1-CD is decreased when the amino acid Y60 is changed to a phenylalanine (F) (MUC1-CD(Y60F)). Compared to MUC1-CD, c-Abl phosphorylation was substantially decreased with the MUC1-CD(Y60F) mutant (FIG. 6D), indicating that c-Abl phosphorylates MUC1, at least in large part, on Tyr-60 in the cytoplasmic domain.

### Example 5

### c-Abl SH2 Domain Binds to the MUC1 pY⁶⁰TNP Site

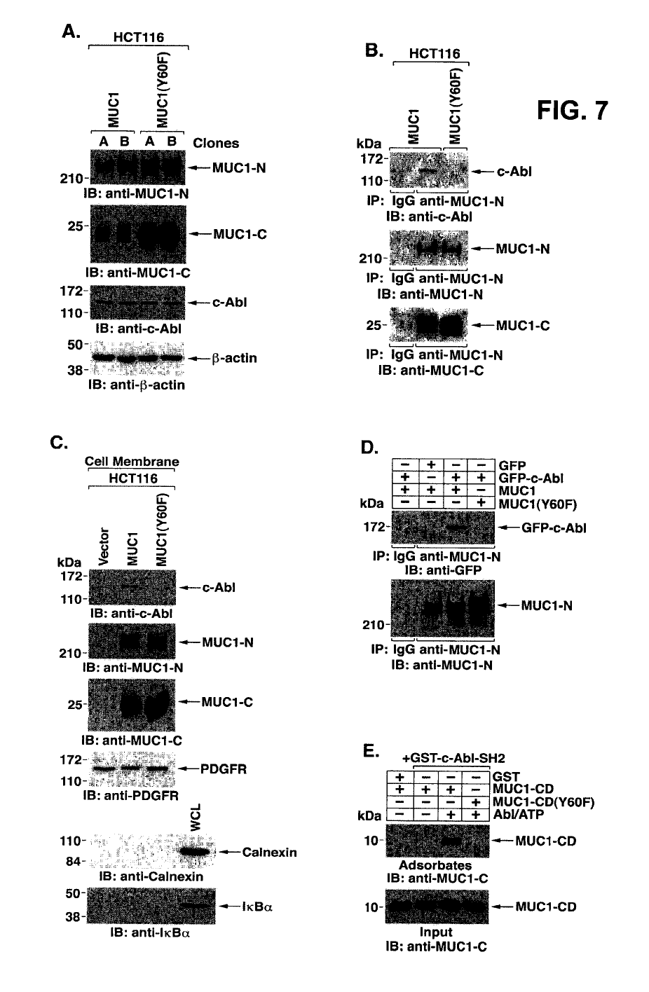

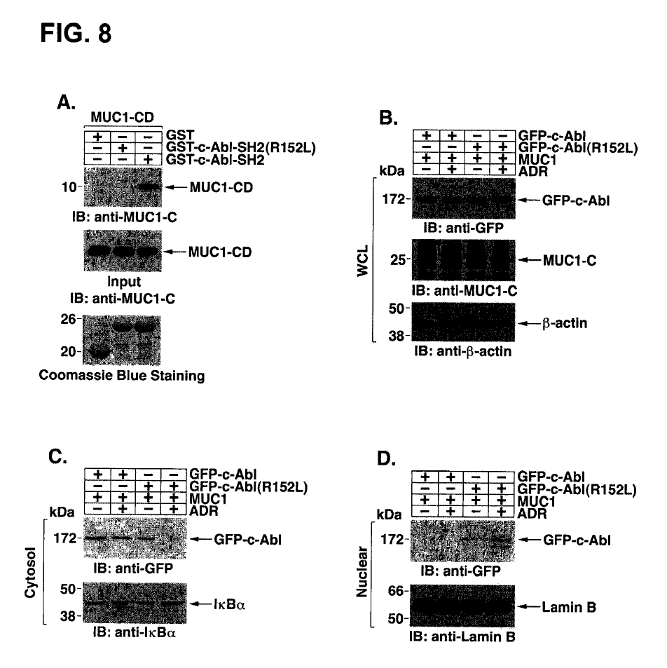

MUC1 with the Y60F mutation was stably expressed in HCT116 cells. Immunoblot analysis, using anti-MUC1 antibodies, of two separately isolated clones showed somewhat higher levels of MUC1(Y60F) as compared to the wild-type protein in stably transfected HCT116/MUC1 cells (FIG. 7A). As found for wild-type MUC1, expression of the MUC1(Y60F) mutant had no effect on c-Abl levels (FIG. 7A). Notably, c-Abl was detectable in anti-MUC1-N immunoprecipitates from HCT116/MUC1 cells, but not HCT116/MUC1(Y60F) cells (FIG. 7B). As a control, the Y60F mutation had no effect on the association between MUC1-N and MUC1-C (FIG. 7B). Similar results were obtained with the two HCT116/MUC1(Y60F) clones. Localization of c-Abl to the cell membrane was also decreased in HCT116/MUC1(Y60F) cells, as compared to that in HCT116/MUC1 cells (FIG. 7C). In 293 cells transiently transfected with expression vectors encoding GFP-c-Abl and MUC1 polypeptides, binding of MUC1 to c-Abl was also attenuated by the Y60F mutation (FIG. 7D). To determine if the pY⁶⁰TNP motif functions as a binding site for the c-Abl SH2 domain, MUC1-CD or MUC1-CD(Y60F) were incubated with kinase-active truncated Abl (no SH2 domain) and ATP. Incubation of the reaction products with GST-c-Abl SH2 demonstrated that binding of the c-Abl SH2 domain to phosphorylated MUC1-CD is abrogated by the Y60F mutation (FIG. 7E). To extend these results, a c-Abl SH2 point mutant was generated in which amino acid arginine-152 (R152) in the conserved FLVRES sequence (SEQ ID NO:6) was modified to leucine (L) (R152L). In vitro, GST-pull-down studies demonstrated that the R152L mutation abrogates binding of GST-c-Abl SH2 to c-Abl phosphorylated MUC1-CD (FIG. 8A). MUC1 was also found to be effective in blocking nuclear targeting of GFP-c-Abl, but not GFP-c-Abl(R152L), in the response to DNA damage (FIGS. 8B-8D). These findings indicate that MUC1 and c-Abl interact directly through binding of the c-Abl SH2 domain to the MUC1 pYTNP motif.

### Example 6

### MUC1 RSVpT⁷³⁵LP Blocks Binding of c-Abl to 14-3-3 Proteins

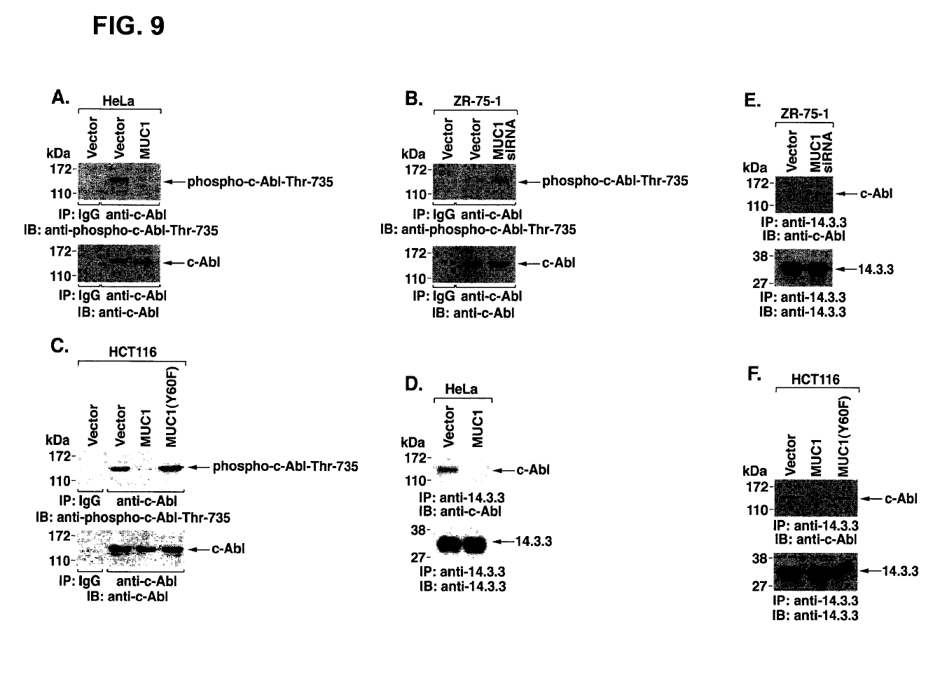

c-Abl contains a RSVT⁷³⁵LP (SEQ ID NO:7) sequence that, when phosphorylated at threonine 735 (the phosphorylated motif is hereinafter referred to as RSVpT^{73.5}LP), confers binding of the Abl polypeptide to 14-3-3 proteins as a mechanism for regulating nuclear targeting of c-Abl in the response to DNA damage (Yoshida et al., (2005) Nat. Cell. Biol. 22:3292-3330). To determine if MUC1 affects phosphorylation of Thr-735 of c-Abl, anti-c-Abl precipitates from HeLa/vector and HeLa/MUC1 cells were immunoblotted with an anti-phospho-c-Abl-Thr-735 antibody. As shown in FIG. 9A, the results demonstrate that MUC1 blocks c-Abl Thr-735 phosphorylation. c-Abl Thr-735 phosphorylation was increased in ZR-75-1 cells expressing MUC1 siRNA (ZR-75-1/MUC1siRNA) (FIG. 9B). In addition, c-Abl Thr-735 phosphorylation was attenuated by expression of MUC1, but not MUC1(Y60F), in HCT116 cells, indicating that the interaction between MUC1 and c-Abl blocks phosphorylation of the Thr-735 site (FIG. 9C). Consistent with a block in Thr-735 phosphorylation, we found that MUC1 inhibits the association between c-Abl and 14-3-3 in HeLa cells (FIG. 9D) and HCT116 cells (FIG. 9F). By contrast, MUC1(Y60F) had little effect on binding of c-Abl to 14-3-3 (FIG. 6F). Further consistent with these results, loss of MUC1 in ZR-75-1 cells resulted in an increased association between c-Abl to 14-3-3 (FIG. 9E). These results indicate that binding of MUC1 and c-Abl blocks phosphorylation of c-Abl on Thr-735 and thereby the association between c-Abl and 14-3-3.

### Example 7

### Binding of MUC1 and c-Abl Attenuates DNA Damage-Induced Apoptosis

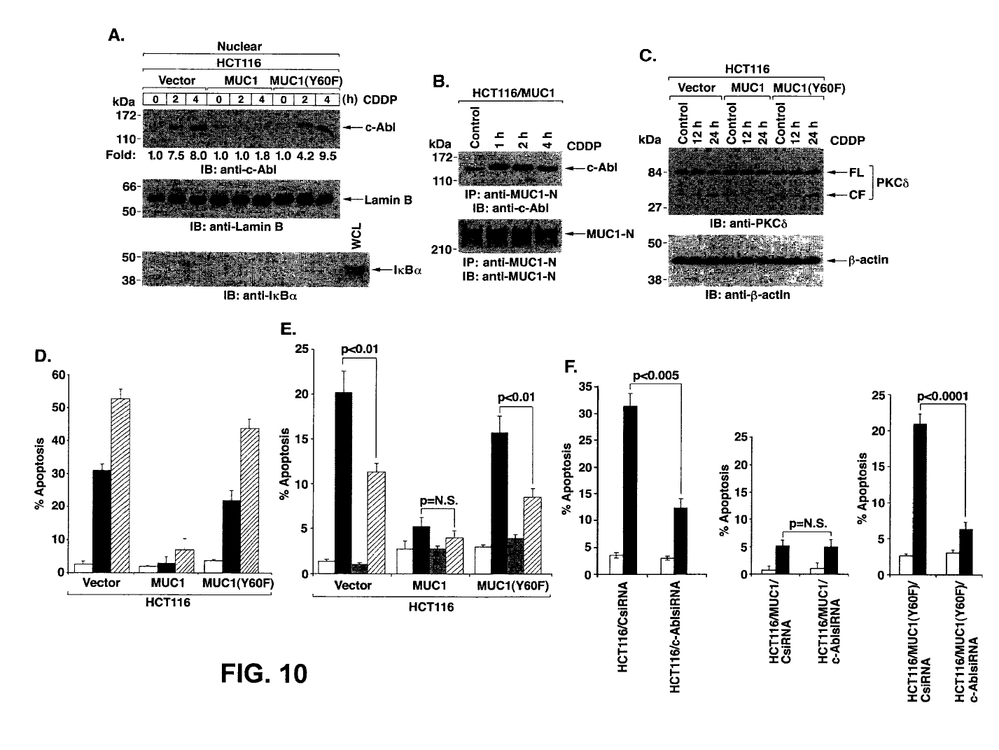

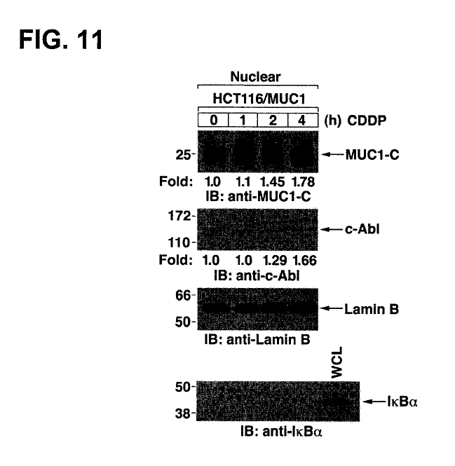

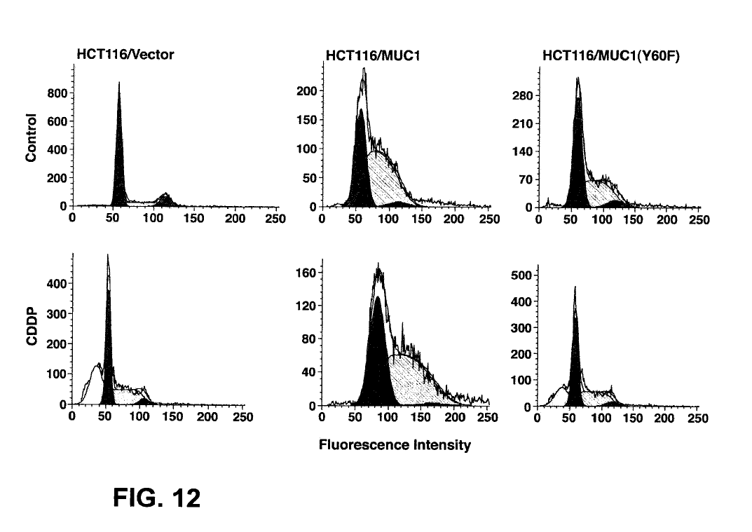

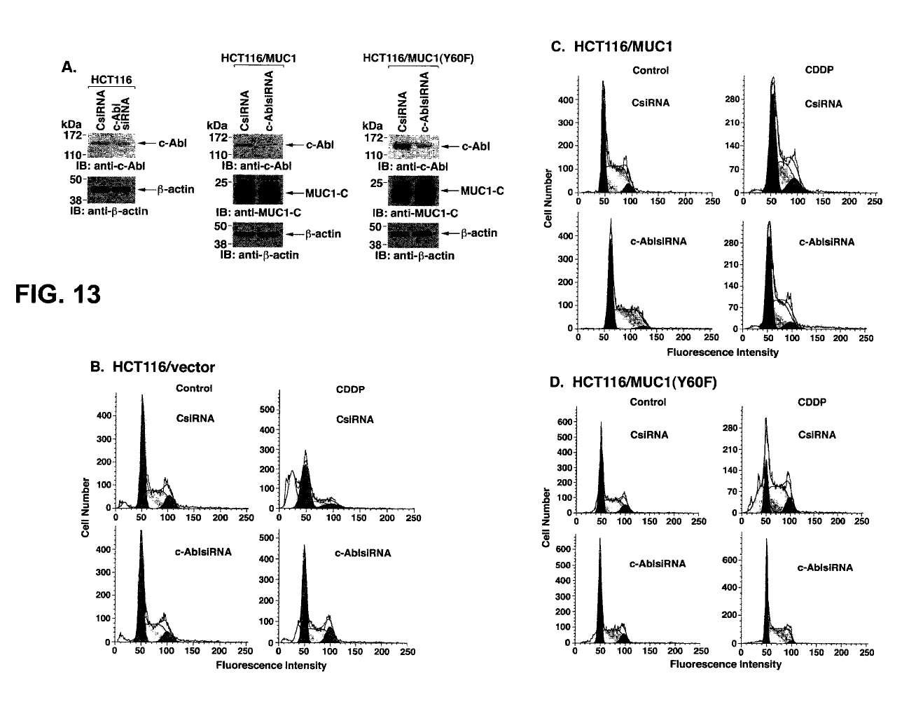

HCT116/vector cells express a low level of nuclear c-Abl and, in the response to CDDP treatment, c-Abl was targeted to the nucleus (FIG. 10A). As found in the other cell types, expression of MUC1 in HCT116 cells blocked nuclear targeting of c-Abl (FIG. 10A). Moreover, there was less than a two-fold increase in nuclear MUC1-C levels in response to CDDP, consistent with the effects on c-Abl (FIG. 11). However, MUC1(Y60F) had little if any effect on CDDP-induced targeting of c-Abl to the nucleus (FIG. 10A). Consistent with the block in nuclear targeting of c-Abl by wild-type MUC1, CDDP treatment increased binding of MUC1, but not MUC1(Y60F), with c-Abl (FIG. 10B). DNA damage activates the intrinsic apoptotic pathway with caspase-3-mediated cleavage of protein kinase Cδ (Emoto et al., (1995) EMBO J. 14:6148-6156); Ghayur et al., (1996) J. Exp. Med. 184:2399-2404). Cleavage of PKCδ was attenuated by MUC1, but not the MUC1(Y60F) mutant (FIG. 10C). Analysis of the cells for sub-G1 DNA further demonstrated that MUC1 blocks DNA damage-induced apoptosis and that this inhibition is relieved by the Y60F mutation (FIG. 12). Similar results were obtained in repeat experiments (FIG. 10D) and when the HCT116 cells were treated with ADR. To assess the role of c-Abl in the induction of apoptosis, cells were treated with CDDP in the absence and presence of STI571, an inhibitor of the c-Abl kinase function (Druker et al., (1996) Nat. Med. 2:561-566). STI751 significantly attenuated CDDP-induced apoptosis in HCT116/vector and HCT116/MUC1(Y60F) cells, but had no significant effect on HCT116/MUC1 cells (FIG. 10E). To substantiate these results, c-Abl expression was downregulated in HCT116/vector, HCT116/MUC1 and HCT116/MUC1(Y60F) cells by transient transfection with a pool of c-Abl siRNAs (FIG. 13A). Consistent with the effects of STI571, c-Abl downregulation was associated with attenuation of CDDP-induced apoptosis in HCT116/vector and HCT116/MUC1(Y60F) cells (FIGS. 10F, 13B and 13D). By contrast, silencing c-Abl in HCT116/MUC1 cells had no significant effect on the induction of apoptosis (FIGS. 10F, 13C). These findings demonstrate that binding of MUC1 to c-Abl and sequestration of c-Abl in the cytosol blocks the proapoptotic function of c-Abl in the response to DNA damage.

### Example 8

### Selectivity of the MUC1 Tyr-60 Site for the Regulation of c-Abl

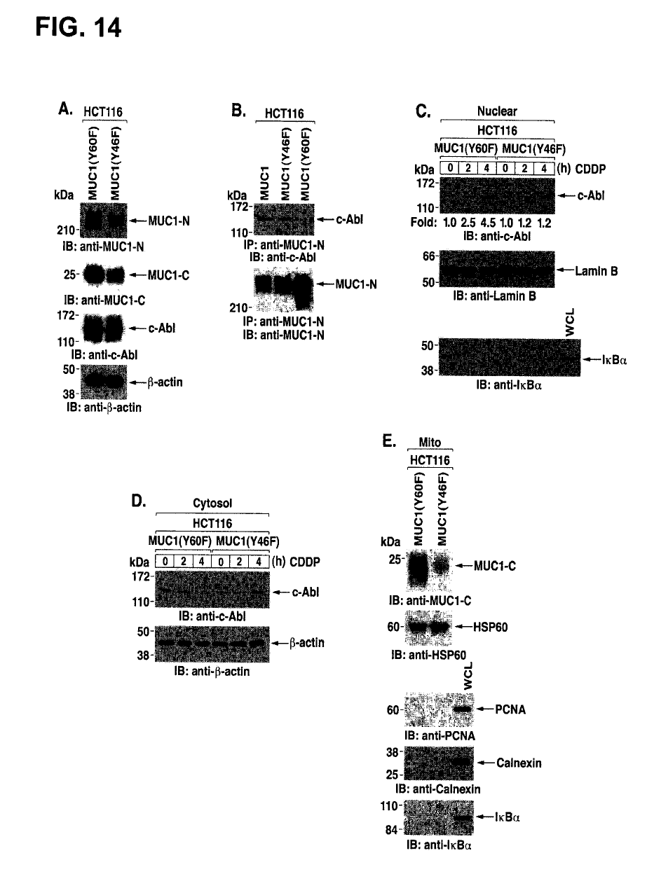

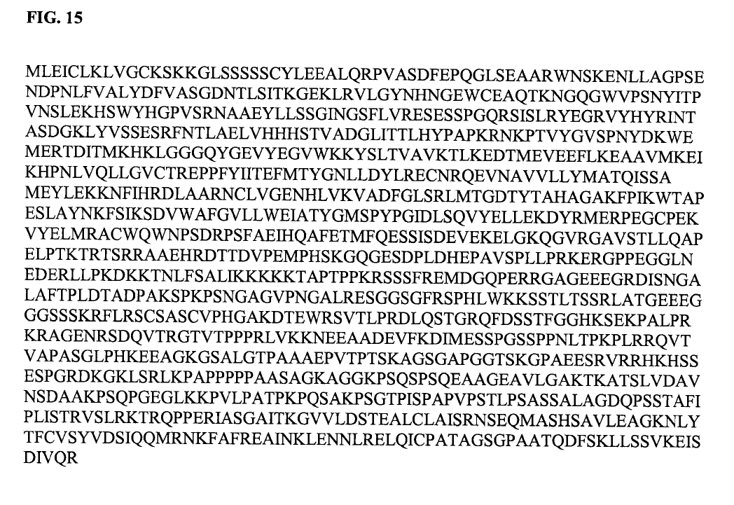

The anti-apoptotic effects of MUC1 are attenuated by mutation of Tyr-46 (Ren et al., (2004) Cancer Cell 5:163-175). To determine whether the loss of MUC1 function by the MUC1(Y60F) and MUC1(Y46F) mutants is due to similar or distinct mechanisms, we compared the effects of stably expressing these proteins in HCT116 cells (wild-type HCT116/MUC1, HCT116/MUC1(Y60F), or HCT116/MUC1(Y46F)) (FIG. 14A). As found for MUC1(Y60F), the MUC1(Y46F) mutant had no apparent effect on c-Abl levels (FIG. 14A). Communoprecipitation studies further demonstrated that, like MUC1, the MUC1(Y46F) mutant associates with c-Abl (FIG. 14B). Moreover, in contrast to MUC1(Y60F), MUC1(Y46F) blocked CDDP-induced targeting of cytosolic c-Abl to the nucleus (FIGS. 14C and 14D). MUC1 localizes to the outer mitochondrial membrane and blocks the release of apoptogenic factors (Ren et al., (2004) Cancer Cell 5:163-175; Ren et al., (2006) Oncogene 25:20-31). In addition, mitochondrial localization of MUC1 is substantially attenuated by mutation of Tyr-46 (Ren et al., (2004) Cancer Cell 5:163-175; Ren et al., (2006) Oncogene 25:20-31), but not the Tyr-60 site (FIG. 14E). These findings indicate that MUC1 Tyr-60, and not Tyr-46, regulates c-Abl function. Conversely, MUC1 Tyr-46, and not Tyr-60, regulates mitochondrial localization.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention.

What is claimed is:

1. A method of identifying a compound that inhibits the binding of Abl to MUC1 (mucin 1), the method comprising:
providing a MUC1 test agent;
providing an Abl test agent that binds to the MUC1 test agent;
contacting the MUC1 test agent with the Abl test agent in the presence of a test compound; and
determining whether the test compound inhibits binding of the MUC1 test agent to the Abl test agent.

2. The method of claim 1, wherein the contacting is carried out in a cell-free system.

3. The method of claim 1, wherein the contacting occurs in a cell.

4. The method of claim 1, wherein the MUC1 test agent comprises the MUC1 cytoplasmic domain.

5. The method of claim 1, wherein the Abl test agent comprises c-Abl.

6. The method of claim 1, wherein the Abl test agent comprises BCR-Abl.

7. A method of generating a compound that inhibits the interaction between MUC1 and an Abl polypeptide, the method comprising:
providing the three-dimensional structure of a molecule comprising: (a) the cytoplasmic domain of MUC1 or an Abl polypeptide-binding fragment thereof; or (b) a molecule comprising an Abl polypeptide or MUC1-binding fragment thereof;
designing, based on the three dimensional structure, a compound comprising a region that inhibits the interaction between MUC1 and the Abl polypeptide; and
producing the compound.

8. The method of claim 7, further comprising determining if the compound inhibits the interaction between MUC1 and an Abl polypeptide.

9. The compound identified by the method of claim 8.

10. An in vitro method of inhibiting an interaction between MUC1 and Abl, the method comprising:
identifying a cell as expressing MUC1; and
culturing the cell with a compound that inhibits an interaction between MUC1 and Abl.

11. An in vivo method of inhibiting an interaction between MUC1 and Abl, the method comprising:
identifying a subject as having, or at risk of developing, a cancer comprising one or more cells expressing MUC1; and
delivering to the subject a compound that inhibits an interaction between MUC1 and Abl.

12. The method of claim 11, wherein the Abl is c-Abl.

13. The method of claim 11, wherein the Abl is BCR-Abl.

14. The method of claim 11, wherein the compound comprises a small molecule, an antibody, an antibody fragment, a polypeptide, or a peptidomimetic.

15. The method of claim 11, wherein the compound comprises a MUC1-binding fragment of Abl.

16. The method of claim 11, wherein the compound comprises a MUC1 fragment comprising tyrosine 60 of MUC1-CD, and wherein the tyrosine is phosphorylated.

17. The method of claim 11, wherein the compound comprises the pYTNP motif of MUC1-CD.

18. The method of claim 11, wherein the cancer is selected from the group consisting of lung cancer, breast cancer, colon cancer, pancreatic cancer, renal cancer, stomach cancer, liver cancer, bone cancer, hematological cancer, neural tissue cancer, melanoma, thyroid cancer, ovarian cancer, testicular cancer, prostate cancer, cervical cancer, vaginal cancer, and bladder cancer.

19. The method of claim 11, further comprising exposing the subject to one or more additional genotoxic agents.

20. The method of claim 19, wherein the one or more additional genotoxic agents comprise one or more chemotherapeutic agents or one or more forms of ionizing radiation.

21. The method of claim 20, wherein the one or more chemotherapeutic agents are selected from the group consisting of carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide, podophyllotoxin, taxol, satraplatinum, 5-fluorouracil, vincristin, vinblastin, methotrexate, ara-C, taxotere, gencitabine, and an analog of any of the aforementioned.

22. The method of claim 19, wherein the one or more genotoxic agents comprise cisplatin (CDDP) or adriamycin (ADR).

23. The method of claim 11, further comprising delivering to the subject one or more kinase inhibitors.

24. The method of claim 23, wherein the one or more kinase inhibitors are selected from the group consisting of trastuzumab, gefitinib, erlotinib, imatinib mesylate, and sunitinib malate.

25. The method of claim 11, wherein the subject is a mammal.

26. The method of claim 25, wherein the mammal is a human.

27. An in vivo method of inhibiting phosphorylation of MUC1 by Abl kinase, the method comprising:
identifying a subject as having, or at risk of developing, a cancer comprising one or more cells expressing MUC1; and
delivering to the subject a compound that inhibits the phosphorylation of MUC1 by Abl kinase.

28. The method of claim 27, wherein the compound comprises a small molecule, an antibody, an antibody fragment, a polypeptide, or a peptidomimetic.

29. The method of claim 27, wherein the compound comprises a MUC1-binding fragment of Abl.

30. The method of claim 27, wherein the compound comprises a MUC1 fragment comprising tyrosine 60 of MUC1-CD.
